SEQUENCE LISTING (208 sequences)

SEQ ID NO: 1 - protein, 303 aa, Homo sapiens
  source 1..303: note = Human PILRA
MGRPLLLPLLPLLLPPAFLQPSGSTGSGPSYLYGVTQPKHLSASMGGSVEIPFSFYYPWELATAPDVRISWRRGHFHRQSFYSTRPPSIHKDYVNRLFLNWTEGQKSGFLRISNLQKQDQSVYFCRVELDTRSSGRQQWQSIEGTKLSITQAVTTTTQRPSSMTTTWRLSSTTTTTGLRVTQGKRRSDSWHISLETAVGVAVAVTVLGIMILGLICLLRWRRRKGQQRTKATTPAREPFQNTEEPYENIRNEGQNTDPKLNPKDDGIVYASLALSSSTSPRAPPSHRPLKSPQNETLYSVLKA

SEQ ID NO: 2 - protein, 307 aa, Macaca fascicularis
  source 1..307: note = Cynomolgus monkey PILRA
MGRPLLLPLLLPLLPLLLPPAFLQPGGSAGSGPSGPYGVTQRKHLSAPMGGSVEIPFSFYHPWELAAAPNMKISWRRGNFHGEFFYRTRPAFIHEDYSNRLLLNWTEGQDRGLLRIWNLRKEDQSVYFCRVELDTRRSGRQRWQSIEGTKLTITQAVTTTTQRPSSMTTTRRPSSATTTAGLRVTQGKRHSDSWHLSLKTAVGVTVAVAVLGIMILGLICLLRWRRRKGQQRTKATTPAKEPFQNTEEPYENIRNEGQNTDPKPNPKDDGIVYASLALSSSTSPRVPPSHHPLKSPQNETLYSVLKV

SEQ ID NO: 3 - protein, 302 aa, synthetic construct
  source 1..302: note = Murine PILRA
MALLISLPGGTPAMAQILLLLSSACLHAGNSERSNRKNGFGVNQPESCSGVQGGSIDIPFSFYFPWKLAKDPQMSIAWRWKDFHGEFIYNSSLPFIHEHFKGRLILNWTQGQTSGVLRILNLKESDQTRYFGRVFLQTTEGIQFWQSIPGTQLNVTNATCTPTTLPSTTAATSAHTQNDITEVKSANIGGLDLQTTVGLATAAAVFLVGVLGLIVFLWWKRRRQGQKTKAEIPAREPLETSEKHESVGHEGQCMDPKENPKDNNIVYASISLSSPTSPGTAPNLPVHGNPQEETVYSIVKAK

SEQ ID NO: 4 - protein, 5 aa, synthetic construct
  source 1..5: note = VH CDR1
NYDVN

SEQ ID NO: 5 - protein, 17 aa, synthetic construct
  source 1..17: note = VH CDR2
GIIPIFGTANYAQKFQG

SEQ ID NO: 6 - protein, 9 aa, synthetic construct
  source 1..9: note = VH CDR3
GVGAGWFDP

SEQ ID NO: 7-9 - skipped (3 entries)

SEQ ID NO: 10 - protein, 5 aa, synthetic construct
  source 1..5: note = VH CDR1
HYDVN

SEQ ID NO: 11 - protein, 9 aa, synthetic construct
  source 1..9: note = VH CDR3
AVGAGWFDT

SEQ ID NO: 12 - protein, 9 aa, synthetic construct
  source 1..9: note = VH CDR3
AVGAGWFDP

SEQ ID NO: 13 - protein, 17 aa, synthetic construct
  source 1..17: note = VL CDR1
KSSQSVLYSSNNKNYLA

SEQ ID NO: 14 - protein, 7 aa, synthetic construct
  source 1..7: note = VL CDR2
WASTRES

SEQ ID NO: 15 - protein, 9 aa, synthetic construct
  source 1..9: note = VL CDR3
QQYYSTPYT

SEQ ID NO: 16 - protein, 11 aa, synthetic construct
  source 1..11: note = VL CDR1
QASQDISQFLN

SEQ ID NO: 17 - protein, 7 aa, synthetic construct
  source 1..7: note = VL CDR2
DAYSLQS

SEQ ID NO: 18 - protein, 9 aa, synthetic construct
  source 1..9: note = VL CDR3
QQANSFPLT

SEQ ID NO: 19 - protein, 17 aa, synthetic construct
  source 1..17: note = VL CDR1
KSSQSVYFSPNNKNYLA

SEQ ID NO: 20 - protein, 17 aa, synthetic construct
  source 1..17: note = VL CDR1
KSSQSVFFDVNNKNYLA

SEQ ID NO: 21 - protein, 17 aa, synthetic construct
  source 1..17: note = VL CDR1
KSSQSVYFDVNNKNYLA

SEQ ID NO: 22 - protein, 17 aa, synthetic construct
  source 1..17: note = VL CDR1
KSSQSVFFDENNKNYLA

SEQ ID NO: 23-24 - skipped (2 entries)

SEQ ID NO: 25 - protein, 9 aa, synthetic construct
  source 1..9: note = VL CDR3
QQYFEEPYT

SEQ ID NO: 26 - protein, 9 aa, synthetic construct
  source 1..9: note = VL CDR3
QQYYEEPYT

SEQ ID NO: 27 - protein, 17 aa, synthetic construct
  source 1..17: note = VL CDR1
KSSQSVWFSPNNKNYLA

SEQ ID NO: 28 - protein, 118 aa, synthetic construct
  source 1..118: note = VH
QVQLVQSGAEVKKPGSSVKVSCKASGYNFLNYDVNWVRQAPGQGLEWMGGIIPIFGTANYAQKFQGRVTITADESTSTAYMELSSLRSEDTAVYYCATGVGAGWFDPWGQGTLVTVSS

SEQ ID NO: 29 - skipped

SEQ ID NO: 30 - protein, 118 aa, synthetic construct
  source 1..118: note = VH
QVQLVQSGAEVKKPGSSVKVSCKASGYNFLHYDVNWVRQAPGQGLEWMGGIIPIFGTANYAQKFQGRVTITADESTSTAYMELSSLRSEDTAVYYCATAVGAGWFDTWGQGTLVTVSS

SEQ ID NO: 31 - protein, 118 aa, synthetic construct
  source 1..118: note = VH
QVQLVQSGAEVKKPGSSVKVSCKASGYNFLHYDVNWVRQAPGQGLEWMGGIIPIFGTANYAQKFQGRVTITADESTSTAYMELSSLRSEDTAVYYCATAVGAGWFDPWGQGTLVTVSS

SEQ ID NO: 32 - protein, 17 aa, synthetic construct
  source 1..17: note = VL CDR1
KSSQSVYFSENNKNYLA

SEQ ID NO: 33 - protein, 17 aa, synthetic construct
  source 1..17: note = VL CDR1
KSSQSVWFDPNNKNYLA

SEQ ID NO: 34-37 - skipped (4 entries)

SEQ ID NO: 38 - protein, 5 aa, synthetic construct
  source 1..5: note = VH CDR1
DYYIH

SEQ ID NO: 39 - protein, 10 aa, synthetic construct
  source 1..10: note = VH CDR3
EGSSGWYVDY

SEQ ID NO: 40-49 - skipped (10 entries)

SEQ ID NO: 50 - protein, 118 aa, synthetic construct
  source 1..118: note = VH
QVQLVQSGAEVKKPGSSVKVSCKASGYNFLHYDVNWVRQAPGQGLEWMGGIIPIFGTANYAQKFQGRVTITADESTSTAYMELSSLRSEDTAVYYCATGVGAGWFDPWGQGTLVTVSS

SEQ ID NO: 51-53 - skipped (3 entries)

SEQ ID NO: 54 - protein, 118 aa, synthetic construct
  source 1..118: note = VH
QVQLVQSGAEVKKPGSSVKVSCKASGYNFLNYDVNWVRQAPGQGLEWMGGIIPIFGTANYAQKFQGRVTITADESTSTAYMELSSLRSEDTAVYYCATAVGAGWFDPWGQGTLVTVSS

SEQ ID NO: 55-57 - skipped (3 entries)

SEQ ID NO: 58 - protein, 17 aa, synthetic construct
  source 1..17: note = VH CDR2
WMNPNSGNTGYAQKFQG

SEQ ID NO: 59 - protein, 107 aa, synthetic construct
  source 1..107: note = VL
DIQMTQSPSSLSASVGDRVTITCQASQDISQFLNWYQQKPGKAPKLLIYDAYSLQSGVPSRFSGSGSGTDFTLTISSLQPEDFATYYCQQANSFPLTFGQGTKVEIK

SEQ ID NO: 60 - protein, 113 aa, synthetic construct
  source 1..113: note = VL
DIVMTQSPDSLAVSLGERATINCKSSQSVLYSSNNKNYLAWYQQKPGQPPKLLIYWASTRESGVPDRFSGSGSGTDFTLTISSLQAEDVAVYYCQQYYSTPYTFGQGTRLEIK

SEQ ID NO: 61-62 - skipped (2 entries)

SEQ ID NO: 63 - protein, 113 aa, synthetic construct
  source 1..113: note = VL
DIVMTQSPDSLAVSLGERATINCKSSQSVLYSSNNKNYLAWYQQKPGQPPKLLIYWASTRESGVPDRFSGSGSGTDFTLTISSLQAEDVAVYYCQQYFEEPYTFGQGTRLEIK

SEQ ID NO: 64 - protein, 113 aa, synthetic construct
DIVMTQSPDSLAVSLGERATINCKSSQSVYFSPNNKNYLAWYQQKPGQPPKLLIYWASTRESGVPDRFSGSGSGTDFTLTISSLQAEDVAVYYCQQYFEEPYTFGQGTRLEIK

SEQ ID NO: 65 - protein, 113 aa, synthetic construct
  source 1..113: note = VL
DIVMTQSPDSLAVSLGERATINCKSSQSVFFDVNNKNYLAWYQQKPGQPPKLLIYWASTRESGVPDRFSGSGSGTDFTLTISSLQAEDVAVYYCQQYFEEPYTFGQGTRLEIK

SEQ ID NO: 66 - protein, 113 aa, synthetic construct
  source 1..113: note = VL
DIVMTQSPDSLAVSLGERATINCKSSQSVYFDVNNKNYLAWYQQKPGQPPKLLIYWASTRESGVPDRFSGSGSGTDFTLTISSLQAEDVAVYYCQQYFEEPYTFGQGTRLEIK

SEQ ID NO: 67 - protein, 113 aa, synthetic construct
  source 1..113: note = VL
DIVMTQSPDSLAVSLGERATINCKSSQSVFFDENNKNYLAWYQQKPGQPPKLLIYWASTRESGVPDRFSGSGSGTDFTLTISSLQAEDVAVYYCQQYFEEPYTFGQGTRLEIK

SEQ ID NO: 68 - protein, 113 aa, synthetic construct
  source 1..113: note = VL
DIVMTQSPDSLAVSLGERATINCKSSQSVWFSPNNKNYLAWYQQKPGQPPKLLIYWASTRESGVPDRFSGSGSGTDFTLTISSLQAEDVAVYYCQQYFEEPYTFGQGTRLEIK

SEQ ID NO: 69 - protein, 113 aa, synthetic construct
  source 1..113: note = VL
DIVMTQSPDSLAVSLGERATINCKSSQSVFFDVNNKNYLAWYQQKPGQPPKLLIYWASTRESGVPDRFSGSGSGTDFTLTISSLQAEDVAVYYCQQYYEEPYTFGQGTRLEIK

SEQ ID NO: 70 - protein, 113 aa, synthetic construct
  source 1..113: note = VL
DIVMTQSPDSLAVSLGERATINCKSSQSVFFDENNKNYLAWYQQKPGQPPKLLIYWASTRESGVPDRFSGSGSGTDFTLTISSLQAEDVAVYYCQQYYEEPYTFGQGTRLEIK

SEQ ID NO: 71 - protein, 113 aa, synthetic construct
  source 1..113: note = VL
DIVMTQSPDSLAVSLGERATINCKSSQSVYFSENNKNYLAWYQQKPGQPPKLLIYWASTRESGVPDRFSGSGSGTDFTLTISSLQAEDVAVYYCQQYYEEPYTFGQGTRLEIK

SEQ ID NO: 72-77 - skipped (6 entries)

SEQ ID NO: 78 - protein, 113 aa, synthetic construct
  source 1..113: note = VL
DIVMTQSPDSLAVSLGERATINCKSSQSVWFDPNNKNYLAWYQQKPGQPPKLLIYWASTRESGVPDRFSGSGSGTDFTLTISSLQAEDVAVYYCQQYFEEPYTFGQGTRLEIK

SEQ ID NO: 79-81 - skipped (3 entries)

SEQ ID NO: 82 - protein, 113 aa, synthetic construct
  source 1..113: note = VL
DIVMTQSPDSLAVSLGERATINCKSSQSVLYSSNNKNYLAWYQQKPGQPPKLLIYWASTRESGVPDRFSGSGSGTDFTLTISSLQAEDVAVYYCQQYYEEPYTFGQGTRLEIK

SEQ ID NO: 83 - protein, 113 aa, synthetic construct
  source 1..113: note = VL
DIVMTQSPDSLAVSLGERATINCKSSQSVYFSPNNKNYLAWYQQKPGQPPKLLIYWASTRESGVPDRFSGSGSGTDFTLTISSLQAEDVAVYYCQQYYSTPYTFGQGTRLEIK

SEQ ID NO: 84 - protein, 113 aa, synthetic construct
  source 1..113: note = VL
DIVMTQSPDSLAVSLGERATINCKSSQSVYFDVNNKNYLAWYQQKPGQPPKLLIYWASTRESGVPDRFSGSGSGTDFTLTISSLQAEDVAVYYCQQYYSTPYTFGQGTRLEIK

SEQ ID NO: 85-88 - skipped (4 entries)

SEQ ID NO: 89 - protein, 30 aa, synthetic construct
  source 1..30: note = VH FR1
QVQLVQSGAEVKKPGSSVKVSCKASGYNFL

SEQ ID NO: 90 - protein, 14 aa, synthetic construct
  source 1..14: note = VH FR2
WVRQAPGQGLEWMG

SEQ ID NO: 91 - protein, 32 aa, synthetic construct
  source 1..32: note = VH FR3
RVTITADESTSTAYMELSSLRSEDTAVYYCAT

SEQ ID NO: 92 - protein, 11 aa, synthetic construct
  source 1..11: note = VH FR4
WGQGTLVTVSS

SEQ ID NO: 93 - protein, 30 aa, synthetic construct
  source 1..30: note = VH FR1
QVQLVQSGAEVKKPGASVKVSCKASGYTFT

SEQ ID NO: 94 - protein, 32 aa, synthetic construct
  source 1..32: note = VH FR3
RVTMTRDTSTSTVYMELSSLRSEDTAVYYCAR

SEQ ID NO: 95 - protein, 23 aa, synthetic construct
  source 1..23: note = VL FR1
DIVMTQSPDSLAVSLGERATINC

SEQ ID NO: 96 - protein, 15 aa, synthetic construct
  source 1..15: note = VL FR2
WYQQKPGQPPKLLIY

SEQ ID NO: 97 - protein, 32 aa, synthetic construct
  source 1..32: note = VL FR3
GVPDRFSGSGSGTDFTLTISSLQAEDVAVYYC

SEQ ID NO: 98 - protein, 10 aa, synthetic construct
  source 1..10: note = VL FR4
FGQGTRLEIK

SEQ ID NO: 99 - protein, 23 aa, synthetic construct
  source 1..23: note = VL FR1
DIQMTQSPSSLSASVGDRVTITC

SEQ ID NO: 100 - protein, 15 aa, synthetic construct
  source 1..15: note = VL FR2
WYQQKPGKAPKLLIY

SEQ ID NO: 101 - protein, 32 aa, synthetic construct
  source 1..32: note = VL FR3
GVPSRFSGSGSGTDFTLTISSLQPEDFATYYC

SEQ ID NO: 102 - protein, 10 aa, synthetic construct
  source 1..10: note = VL FR4
FGQGTKVEIK

SEQ ID NO: 103-104 - skipped (2 entries)

SEQ ID NO: 105 - protein, 227 aa, synthetic construct
  source 1..227: note = human PILRB
MGRPLLLPLLLLLQPPAFLQPGGSTGSGPSYLYGVTQPKHLSASMGGSVEIPFSFYYPWELAIVPNVRISWRRGHFHGQSFYSTRPPSIHKDYVNRLFLNWTEGQESGFLRISNLRKEDQSVYFCRVELDTRRSGRQQLQSIKGTKLTITQAVTTTTTWRPSSTTTIAGLRVTESKGHSESWHLSLDTAIRVALAVAVLKTVILGLLCLLLLWWRRRKGSRAPSSDF

SEQ ID NO: 106 - protein, 232 aa, synthetic construct
  source 1..232: note = human IgG1 Fc domain
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 107 - protein, 232 aa, synthetic construct
  source 1..232: note = human IgG1 Fc
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSSKAFPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 108 - protein, 232 aa, synthetic construct
  source 1..232: note = human IgG1 Fc
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 109 - protein, 229 aa, synthetic construct
  source 1..229: note = human IgG4 Fc
ESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 110 - protein, 229 aa, synthetic construct
  source 1..229: note = human IgG4 Fc
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 111-119 - skipped (9 entries)

SEQ ID NO: 120 - protein, 294 aa, Macaca fascicularis
  source 1..294: note = Cynomolgus monkey PILRA
MGRPLLLPLLPLLLPPAFLQPGGSAGSGPSGPYGVTQRKHLSAPMGGSVEIPFSFYHPWELAAAPNMKISWRRGNFHGEFFYRTRPAFIHEDYSNRLLLNWTEGQDRGLLRIWNLRKEDQSVYFCRVELDTRRSGRQRWQSIEGTKLTITQAVTTTTRRPSSATTTAGLRVTQGKRHSDSWHLSLKTAVGVTVAVAVLGIMILGLICLLRWRRRKGQQRTKATTPAKEPFQNTEEPYENIRNEGQNTDPKPNPKDDGIVYASLALSSSTSPRVPPSHHPLKSPQNETLYSVLKV

SEQ ID NO: 121 - protein, 263 aa, Macaca fascicularis
  source 1..263: note = cynomolgus monkey PILRB
MGRPLLLPLLPLLLPPAFLQPGGSAGSGPSGPYGVTQRKHLSAPMGGSVEIPFSFYHPWELAAAPNMKISWRRGNFHGEFFYRTRPAFIHEDYSNRLLLNWTEGQDRGLLRIWNLRKEDQSVYFCRVELDTRRSGMQRLQSIEGTKLTITQAVMTTTTWRPSSATTTAGLRVTEGKGHSESWHLSLDTAISVALAVAVLKTVTLGLLCLLRWKRRKGQQQTKATTPARVWRDGMCISPRGCAVRPACEYSTLAPHWQDTWRAS

SEQ ID NO: 122-139 - skipped (18 entries)

SEQ ID NO: 140 - protein, 17 aa, synthetic construct
  source 1..17: note = VL CDR1
KSSQSVLFDVNNKNYLA

SEQ ID NO: 141 - protein, 10 aa, synthetic construct
  source 1..10: note = VL CDR3
CQQYYEEPYT

SEQ ID NO: 142 - protein, 113 aa, synthetic construct
  source 1..113: note = VL
DIVMTQSPDSLAVSLGERATINCKSSQSVYFDVNNKNYLAWYQQKPGQPPKLLIYWASTRESGVPDRFSGSGSGTDFTLTISSLQAEDVAVYYCQQYYEEPYTFGQGTRLEIK

SEQ ID NO: 143 - protein, 113 aa, synthetic construct
  source 1..113: note = VL
DIVMTQSPDSLAVSLGERATINCKSSQSVLFDVNNKNYLAWYQQKPGQPPKLLIYWASTRESGVPDRFSGSGSGTDFTLTISSLQAEDVAVYYCQQYYEEPYTFGQGTRLEIK

SEQ ID NO: 144 - protein, 31 aa, synthetic construct
  source 1..31: note = VL FR3
GVPDRFSGSGSGTDFTLTISSLQAEDVAVYY

SEQ ID NO: 145-199 - skipped (55 entries)

SEQ ID NO: 200 - protein, 5 aa, synthetic construct
  source 1..5: note = VH CDR1
NYDIN

SEQ ID NO: 201 - protein, 17 aa, synthetic construct
  source 1..17: note = VH CDR2
GIIPIFGAPVYAQDFQG

SEQ ID NO: 202 - protein, 9 aa, synthetic construct
  source 1..9: note = VH CDR3
ADGAGWFDP

SEQ ID NO: 203 - protein, 17 aa, synthetic construct
  source 1..17: note = VL CDR1
KSSQSVFYSSNNQNYLA

SEQ ID NO: 204 - protein, 9 aa, synthetic construct
  source 1..9: note = VL CDR3
QQYYSSPLT

SEQ ID NO: 205 - protein, 118 aa, synthetic construct
  source 1..118: note = VH
QVQLVQSGAEVKKPGSSVKVSCKASGYTFINYDINWVRQAPGQGLEWMGGIIPIFGAPVYAQDFQGRVTITADESTSTAYMELSSLRSEDTAVYYCAIADGAGWFDPWGQGTLVTVSS

SEQ ID NO: 206 - protein, 113 aa, synthetic construct
  source 1..113: note = VL
DIVMTQSPDSLAVSLGERATINCKSSQSVFYSSNNQNYLAWYQQKPGQPPKLLIYWASTRESGVPDRFSGSGSGTDFTLTISSLQAEDVAVYYCQQYYSSPLTFGQGTRLEIK

SEQ ID NO: 207 - protein, 30 aa, synthetic construct
  source 1..30: note = VH FR1
QVQLVQSGAEVKKPGSSVKVSCKASGYTFI

SEQ ID NO: 208 - protein, 32 aa, synthetic construct
  source 1..32: note = VH FR3
RVTITADESTSTAYMELSSLRSEDTAVYYCAI

(19) United States
(12) Patent Application Publication
(10) Pub. No.: US 2026/0070976 A1
(43) Pub. Date: Mar. 12, 2026

(54) PILRA Antibodies and Methods of Use Thereof

(71) Applicant: Alector LLC, South San Francisco, CA (US)

(72) Inventors: Samuel Nalle, Pacifica, CA (US); Ling Leung, Foster City, CA (US); Yong Wang, Alviso, CA (US); Angie Grace Yee, San Francisco, CA (US)

(73) Assignee: Alector LLC, South San Francisco, CA (US)

(21) Appl. No.: 19/160,537

(22) PCT Filed: Feb. 29, 2024
(86) PCT No.: PCT/US2024/017794

§ 371 Date: Aug. 28, 2025

Related U.S. Application Data
(60) Provisional application No. 63/487,731, filed on Mar. 1, 2023.

(51) Int. Cl. C07K 16/28; A61P 37/04

(52) U.S. Cl. CPC C07K 16/2803; A61P 37/04; C07K 16/2827; C07K 2317/14; C07K 2317/33; C07K 2317/52; C07K 2317/565; C07K 2317/71; C07K 2317/76; C07K 2317/92

(57) ABSTRACT

The present disclosure provides antibodies and antigen-binding fragments thereof that specifically bind to human PIL-RA and compositions comprising such antibodies or antigen-binding fragments thereof. In a particular aspect, the antibodies or anti-gen-binding fragments thereof that specifically bind to human PILRA block binding of PILRA to ligand and/or decrease cell surface PILRA. In further aspects, the antibodies or antigen-binding fragments can be used to treat diseases or conditions associated with myeloid cell dysfunction.

### 1. CROSS-REFERENCE TO RELATED APPLICATIONS

This application claims the priority benefit of U.S. Provisional Appl. No. 63/487,731, filed Mar. 1, 2023, which is herein incorporated by reference in its entirety.

### 2. REFERENCE TO SEQUENCE LISTING SUBMITTING ELECTRONICALLY

The content of the electronically submitted sequence listing (Name: 4503_024PC01_Seqlisting_ST26.XML; Size: 117,321 bytes; and Date of Creation: Feb. 21, 2024) is herein incorporated by reference in its entirety.

### 3. FIELD

The present disclosure relates to antibodies that specifically bind to human PILRA, compositions comprising such antibodies, and methods of making and using antibodies that specifically bind to human PILRA.

### 4. DESCRIPTION OF RELATED ART

Paired immunoglobulin-like type 2 receptor alpha (PILRA) is a cell surface receptor that is expressed on various innate immune cells of the myeloid lineage, such as monocytes, macrophages, microglia (in the CNS), dendritic cells and neutrophils. PILRA is an inhibitory receptor containing an intracellular ITIM domain and an extracellular IgV domain, and its ligands include specific sialylated O-glycosylated proteins. PILRA is also the entry receptor for herpes simplex virus 1 (HSV-1). A naturally occurring allele in the PILRA gene results in a missense variant (G78 to R78) in the encoded PILRA protein. The R78 variant of PILRA is associated with reduced risk of Alzheimer's disease. This variant is also reported to reduce binding of PILRA to several of its ligands by altering access to the sialic acid binding pocket of PILRA. It has been proposed that this variant protects individuals from Alzheimer's disease by reducing inhibitory signaling in microglia and reducing microglial infection during HSV-1 recurrence.

Given the expression and function of PILRA, provided herein are antibodies that specifically bind to human PILRA. Such antibodies may reduce inhibitory signaling of PILRA by blocking the binding of PILRA to ligands, blocking binding of soluble PILRA to T cells, and/or downregulating cell surface PILRA. Such antibodies can be used to activate myeloid cells and to treat diseases in which myeloid cell activation is desired, including cancer and neurodegenerative diseases such as Alzheimer's disease. These and other compositions and methods are provided herein.

### 5. SUMMARY

Provided herein are isolated antibodies and antigen-binding fragments thereof that specifically bind to human PILRA and methods of use thereof, compositions comprising the same, and methods of using the antibodies, fragments, and compositions.

In one aspect (A1), provided herein is an isolated antibody or antigen-binding fragment thereof that specifically binds to human PILRA and comprises the heavy chain variable region (VH) complementarity determining region (CDR) 1, VH CDR2, VH CDR3 and light chain variable region (VL) CDR1, CDR2, and CDR3 sequences of: SEQ ID NOs: 4-6, 32, 14, and 26, respectively; SEQ ID NOs: 10, 5, 11, and 13-15, respectively; SEQ ID NOs: 10, 5, 12, and 13-15, respectively; SEQ ID NOs: 4-6, 13, 14, and 25, respectively; SEQ ID NOs: 4-6, 19, 14, and 25, respectively; SEQ ID NOs: 4-6, 20, 14, and 25, respectively; SEQ ID NOs: 4-6, 21, 14, and 25, respectively; SEQ ID NOs: 4-6, 22, 14, and 25, respectively; SEQ ID NOs: 4-6, 27, 14, and 25, respectively; SEQ ID NOs: 4-6, 20, 14, and 26, respectively; SEQ ID NOs: 4-6, 22, 14, and 26, respectively; SEQ ID NOs: 10, 5, 11, 13, 14, and 25, respectively; SEQ ID NOs: 10, 5, 12, 13, 14, and 25, respectively; SEQ ID NOs: 10, 5, 11, 19, 14, and 25, respectively; SEQ ID NOs: 10, 5, 11, 20, 14, and 25, respectively; SEQ ID NOs: 10, 5, 11, 21, 14, and 25, respectively; SEQ ID NOs: 10, 5, 11, 22, 14, and 25, respectively; SEQ ID NOs: 10, 5, 11, 33, 14, and 25, respectively; SEQ ID NOs: 10, 5, 12, 22, 14, and 26, respectively; SEQ ID NOs: 10, 5, 12, 32, 14, and 26, respectively; SEQ ID NOs: 10, 5, 6, 13, 14, and 25, respectively; SEQ ID NOs: 10, 5, 6, 13, 14, and 26, respectively; SEQ ID NOs: 10, 5, 6, 19, 14, and 15, respectively; SEQ ID NOs: 10, 5, 6, 21, 14, and 15, respectively; SEQ ID NOs: 4, 5, 12, 13, 14, and 25, respectively; SEQ ID NOs: 4, 5, 12, 13, 14, and 26, respectively; SEQ ID NOs: 4, 5, 12, 19, 14, and 15, respectively; SEQ ID NOs: 4, 5, 12, 21, 14, and 15, respectively; SEQ ID NOs: 4-6, 21, 14, and 141, respectively; SEQ ID NOs: 4-6, 140, 14, and 141, respectively; SEQ ID NOs: 10, 5, 12, 20, 14, and 141, respectively; or SEQ ID NOs: 110, 5, 12, 140, 14, and 141, respectively.

In one aspect (A2) of A1, the antibody or antigen-binding comprises a heavy chain variable region and/or a light chain variable region comprising the amino acid sequences of: SEQ ID NOs: 28 and 71, respectively; SEQ ID NOs: 30 and 60, respectively; SEQ ID NOs: 31 and 60, respectively; SEQ ID NOs: 28 and 63, respectively; SEQ ID NOs: 28 and 64, respectively; SEQ ID NOs: 28 and 65, respectively; SEQ ID NOs: 28 and 66, respectively; SEQ ID NOs: 28 and 67, respectively; SEQ ID NOs: 28 and 68, respectively; SEQ ID NOs: 28 and 69, respectively; SEQ ID NOs: 28 and 70, respectively; SEQ ID NOs: 30 and 63, respectively; SEQ ID NOs: 31 and 63, respectively; SEQ ID NOs: 30 and 64, respectively; SEQ ID NOs: 30 and 65, respectively; SEQ ID NOs: 30 and 66, respectively; SEQ ID NOs: 30 and 67, respectively; SEQ ID NOs: 30 and 78, respectively; SEQ ID NOs: 31 and 70, respectively; SEQ ID NOs: 31 and 71, respectively; SEQ ID NOs: 50 and 63, respectively; SEQ ID NOs: 50 and 82, respectively; SEQ ID NOs: 50 and 83, respectively; SEQ ID NOs: 50 and 84, respectively; SEQ ID NOs: 54 and 63, respectively; SEQ ID NOs: 54 and 82, respectively; SEQ ID NOs: 54 and 83, respectively; SEQ ID NOs: 54 and 84, respectively; SEQ ID NOs: 28 and 142, respectively; SEQ ID NOs: 28 and 143, respectively; SEQ ID NOs: 31 and 69, respectively; or SEQ ID NOs: 31 and 143, respectively.

In one aspect (A3) of A1 or A2, the antibody or antigen-binding fragment binds to human PILRA with a binding affinity between about 0.2 nM to 12 nM.

In one aspect (A4) of any one of A1-A3, the antibody or antigen-binding fragment blocks binding of human PILRA to one or more of its ligands. In one aspect (A5), of A4, the ligand is NPDC1.

In one aspect (A6) of any one of A1-A5, the antibody or antigen-binding fragment binds to cynomolgus PILRA. In one aspect (A7), of A6, the antibody or antigen-binding fragment binds to cynomolgus PILRA with a binding affinity between about 0.1 nM to 2 nM.

In one aspect (A8) of any one of A1-A7, the antibody or antigen-binding fragment binds to human PILRB with a binding affinity between about 2 nM to 200 nM. In one aspect (A9) of any one of A1-A8, the antibody or antigen-binding fragment binds to cynomolgus PILRB with a binding affinity between about 0.1 nM to 2 nM.

In one aspect (A10) of any one of A1-A9, the antibody or antigen-binding fragment blocks binding of soluble human PILRA binding to T cells.

In one aspect (A11) of any one of A1-A10, the antibody or antigen-binding fragment thereof downregulates cell surface human PILRA. In one aspect (A12) of any one of A1-A11, the antibody or antigen-binding fragment thereof downregulates cell surface human PILRA by about 20% to 35% more than antibody X0. In one aspect (A13) of any one of A1-A11, the antibody or antigen-binding fragment thereof downregulates cell surface human PILRA by at least 20% more than antibody X0. In one aspect (A14) of any one of A1-A12, the antibody or antigen-binding fragment thereof downregulates cell surface human PILRA by at least 25% more than antibody X0. In one aspect (A15) of any one of A1-A12, the antibody or antigen-binding fragment thereof downregulates cell surface human PILRA by at least 30% more than antibody X0. In one aspect (A16) of any one of A1-A11, the antibody or antigen-binding fragment thereof downregulates cell surface human PILRA by at least 35% more than antibody X0.

In one aspect (A17) of any one of A1-A16, the antibody or antigen-binding fragment agonizes human PILRB.

In one aspect (A18) of any one of A1-A17, the antibody or antigen-binding fragment binds to the G78 variant of human PILRA, optionally wherein the antibody or antigen-binding fragment binds to the G78 variant of human PILRA with an EC50 of no more than 4 μg/ml, no more than 3.5 μg/ml, no more than 3 μg/ml, no more than 2.5 μg/ml, no more than 2 μg/ml, no more than 1.5 μg/ml, or no more than 1 μg/ml.

In one aspect (A19) of any one of A1-A18, the antibody or antigen-binding fragment binds to cynomolgus PILRA with an EC50 of no more than 20 μg/ml, no more than 15 μg/ml, no more than 10 μg/ml, no more than 5 μg/ml, no more than 3 μg/ml, or no more than 1 μg/ml.

In one aspect (A20) of any one of A1-A19, the antibody or antigen-binding fragment decreases binding of human PILRA to one or more of its ligands by at least 90%.

In one aspect (A21) of any one of A1-A20, the antibody or antigen-binding fragment thereof is capable of producing a dose-dependent induction of MCP-1 in monocytes. In one aspect (A22) of any one of A1-A21, the antibody or antigen-binding fragment thereof is capable of inducing release of at least 38,000 μg/mL MCP-1, wherein the antibody or antigen-binding fragment is present at a concentration of 1 μg/mL. In one aspect (A23) of any one of A1-A22, the antibody or antigen-binding fragment thereof is capable of inducing more MCP-1 in monocytes than antibody X0. In one aspect (A24) of any one of A1-A22, the antibody or antigen-binding fragment thereof is capable of inducing 10% to 50% more MCP-1 in monocytes than antibody X0. In one aspect (A25) of any one of A1-A22, the antibody or antigen-binding fragment thereof is capable of inducing at least 10% more MCP-1 in monocytes than antibody X0. In one aspect (A26) of any one of A1-A24, the antibody or antigen-binding fragment thereof is capable of inducing at least 20% more MCP-1 in monocytes than antibody X0. In one aspect (A27) of any one of A1-A24, the antibody or antigen-binding fragment thereof is capable of inducing at least 30% more MCP-1 in monocytes than antibody X0. In one aspect (A28) of any one of A1-A24, the antibody or antigen-binding fragment thereof is capable of inducing at least 40% more MCP-1 in monocytes than antibody X0. In one aspect (A29) of any one of A1-A24, the antibody or antigen-binding fragment thereof is capable of inducing at least 50% more MCP-1 in monocytes than antibody X0.

In one aspect (A30) of any one of A1-A29, the antibody or antigen-binding fragment thereof is capable of producing a dose-dependent induction of IL-8 in monocytes.

In one aspect (A31) of any one of A1-A30, the antibody or antigen-binding fragment thereof is capable of inducing IFNg in macrophages.

In one aspect (A32) of any one of A1-A31, the antibody or antigen-binding fragment thereof is capable of inducing TNFa in macrophages.

In one aspect (A33) of any one of A1-A32, the antibody or antigen-binding fragment thereof is capable of inducing IL-6 in macrophages.

In one aspect (A34) of any one of A1-A33, the antibody or antigen-binding fragment thereof in combination with an antagonist of PD-L1 is capable of inducing IFNg in macrophages.

In one aspect (A35) of any one of A1-A34, the antibody or antigen-binding fragment thereof in combination with an antagonist of PD-L1 is are capable of inducing TNFa in macrophages.

In one aspect (A36) of any one of A1-A35, the antibody or antigen-binding fragment thereof and combination with an antagonist of PD-L1 agent is capable of inducing IL-2 in macrophages.

In one aspect (A37) of any one of A1-A36, the antibody or antigen-binding fragment thereof in combination with an antagonist of PD-L1 is capable of inducing IL-6 in macrophages.

In one aspect (A38) of any one of A34-A37, the antagonist of PD-L1 is an anti-PD-L1 antibody or antigen-binding fragment thereof, optionally wherein the anti-PD-L1 antibody or antigen-binding fragment thereof is selected from the group consisting of atezolizumab, durvalumab (MEDI4736), BMS-936559, MSB0010718C and rHigM12B7.

In one aspect (A39) of any one of A1-A38, the antibody or antigen-binding fragment comprises a heavy chain constant region and a light chain constant region. In one aspect (A40) of A39, the heavy chain constant region is an isotype selected from the group consisting of human IgG₁, IgG₂, IgG₃, and IgG₄ isotypes.

In one aspect (A41) of any one of A1-A40, the antibody or antigen-binding fragment comprises comprises an Fc domain that is engineered to reduce effector function.

In one aspect (A42) of any one of A1-A41, the antibody or antigen-binding fragment comprises a heavy chain constant region and a light chain constant region, wherein the heavy chain constant region is a human IgG₁ heavy chain constant region, and wherein the light chain constant region is a human IgG_{κ} light chain constant region.

In one aspect (A43) of any one of A1-A42, the antibody or antigen-binding fragment is a monoclonal antibody.

In one aspect (A44) of any one of A1-A43, the antibody or antigen-binding fragment thereof is a murine, chimeric, humanized, or human antibody or antigen-binding fragment thereof.

In one aspect (A45) of any one of A1-A44, the antibody or antigen-binding fragment is is a full length antibody.

In one aspect (A46) of any one of A1-A44, the antibody or antigen-binding fragment is is an antigen binding fragment.

In one aspect (A47) of A46, the antigen binding fragment is a Fab, Fab′, F(ab′)₂, single chain Fv (scFv), disulfide linked Fv, V-NAR domain, IgNar, intrabody, IgGΔCH2, minibody, F(ab′)₃, tetrabody, triabody, diabody, single-domain antibody, DVD-Ig, Fcab, mAb², (scFv)₂, or scFv-Fc.

In one aspect (A48), of any one of A1-A47, the antibody or antigen-binding fragment thereof further comprises a detectable label.

In one aspect (A49), provided herein is an antibody or antigen-binding fragment thereof that binds to the same epitope as the antibody or antigen-binding fragment thereof of any one of A1-A47.

In one aspect (A50), provided herein is an antibody or antigen-binding fragment thereof that competitively inhibits binding to human PILRA of the antibody or antigen-binding fragment thereof of any one of A1-A47.

In one aspect (A51), provided herein is an isolated polynucleotide comprising a nucleic acid molecule encoding the heavy chain variable region or heavy chain of the antibody or antigen-binding fragment thereof of any one of A1-A50. In one aspect (A52) of A51, the nucleic acid molecule encodes the VH of any one of SEQ ID NOs: 28, 30, 31, 50, or 54.

In one aspect (A53), provided herein is an isolated polynucleotide comprising a nucleic acid molecule encoding the light chain variable region or light chain of the antibody or antigen-binding fragment thereof of any one of A1-A50. In one aspect (A54) of A53, the nucleic acid molecule encodes the VL of any one of SEQ ID NOs: 60, 63-71, 78, 82-84, 142, or 143.

In one aspect (A55), provided herein is an isolated polynucleotide comprising a nucleic acid molecule encoding the heavy chain variable region or heavy chain of the antibody or antigen-binding fragment thereof of any one of A1-A50 and the light chain variable region or light chain of the antibody or antigen-binding fragment thereof of any one of claims A1-A50.

In one aspect (A56), provided herein is an isolated vector comprising the polynucleotide of any one of A51-A55.

In one aspect (A57), provided herein is a host cell comprising (a) the polynucleotide of any one of A51-A55 (b) the vector of A56, or (c) a first vector comprising the polynucleotide of A51 or A52 and a second vector comprising the polynucleotide of A53 or A54. In one aspect (A58) of A57, the host cell is selected from the group consisting of E. coli, Pseudomonas, Bacillus, Streptomyces, yeast, CHO, YB/20, NSO, PER-C6, HEK-293T, NIH-3T3, HeLa, BHK, Hep G2, SP2/0, R1.1, B-W, L-M, COS 1, COS 7, BSC1, BSC40, BMT10 cell, plant cell, insect cell, and human cell in tissue culture, optionally wherein the host cell is a HEK-293 cell.

In one aspect (A59), provided herein is a method of producing an antibody or antigen-binding fragment thereof that binds to human PILRA comprising culturing the host cell of A57 or A58 so that the nucleic acid molecule is expressed and the antibody or antigen-binding fragment thereof is produced, optionally wherein the method further comprises isolating the antibody or antigen-binding fragment thereof from the culture.

In one aspect (A60), provided herein is an isolated antibody or antigen-binding fragment thereof that specifically binds to human PILRA and is encoded by the polynucleotide of any one of A51-A55 or produced by the method of A59.

In one aspect (A61), provided herein is a pharmaceutical composition comprising the antibody or antigen-binding fragment thereof of any one of A1-A50 and A60 and a pharmaceutically acceptable excipient.

In one aspect (A62), provided herein is a method for downregulating cell surface PILRA comprising contacting a cell expressing PILRA on its surface with the antibody or antigen-binding fragment thereof of any one of A1-A50 and A60 or the pharmaceutical composition of A61.

In one aspect (A63), provided herein is a method for inhibiting binding of PILRA to a PILRA ligand comprising contacting PILRA with the antibody or antigen-binding fragment thereof of any one of A1-A50 and A60 or the pharmaceutical composition of A61 in the presence of the PILRA ligand, optionally wherein the PILRA and/or the PILRA ligand is expressed on a cell.

In one aspect (A64) of A63, the PILRA ligand is NPDC1. In one aspect (A65) of A63 or A64, the PILRA ligand is expressed on a T cell.

In one aspect (A66), provided herein is a method for increasing myeloid cell activation comprising contacting the myeloid cell with the antibody or antigen-binding fragment thereof of any one of A1-A50 and A60 or the pharmaceutical composition of A61. In one aspect (A67) of A66, the myeloid cell activation is Fc receptor-mediated.

In one aspect (A68), provided herein is a method for promoting myeloid cell differentiation comprising contacting the myeloid cell with the antibody or antigen-binding fragment thereof of any one of A1-A50 and A60 or the pharmaceutical composition of A61.

In one aspect (A69), provided herein is a method for increasing myeloid cell production of MIP1b comprising contacting the myeloid cell with the antibody or antigen-binding fragment thereof of any one of A1-A50 and A60 or the pharmaceutical composition of A61.

In one aspect (A70) of any one of A62-A69, the contacting is in vitro.

In one aspect (A71) of any one of A62-A69, the contacting is in a subject.

In one aspect (A72), provided herein is a method of treating cancer in a patient, the method comprising administering to the patient a therapeutically effective amount of the antibody or antigen binding fragment thereof of any one of A1-A50 and A60 or the pharmaceutical composition of A61. In one aspect (A73) of A72, the cancer is a solid tumor in which myeloid cells have infiltrated the tumor microenvironment. In one aspect (A74) of A72 or A73, the cancer is selected from the group consisting of: glioblastoma, head and neck cancer, kidney cancer (optionally wherein the kidney cancer is kidney clear cell cancer), pancreatic cancer, breast cancer, ovarian cancer, sarcoma, colorectal cancer, lung cancer, melanoma, bladder cancer, liver cancer, and uterine cancer.

In one aspect (A75), of any one of A72-A74, the method further comprises administering an antagonist of an inhibitory immune checkpoint molecule, optionally wherein the immune checkpoint molecule is PD-1 or PD-L1. In one aspect (A76) of X75, the antagonist is an antagonist of PD-1, which is an anti-PD-1 antibody or antigen-binding fragment thereof, optionally wherein the anti-PD-1 antibody or antigen-binding fragment thereof is selected from the group consisting of nivolumab, pembrolizumab, MEDI-0680 (AMP-514), camrelizumab (SHR-1210), tislelizumab (BGB-A317), and spartalizumab (NPVPDR001, NVS240118, PDR001). In one aspect (A77) of A75, the antagonist is an antagonist of PD-L1, which is an anti-PD-L1 antibody or antigen-binding fragment thereof, optionally wherein the anti-PD-L1 antibody or antigen-binding fragment thereof is selected from the group consisting of atezolizumab, durvalumab (MEDI4736), BMS-936559, MSB0010718C and rHigM12B7.

In one aspect (A78) of any one of A75-A77, the antibody or antigen-binding fragment thereof that specifically binds to human PILRA and the antagonist of the inhibitory immune checkpoint molecule are administered simultaneously.

In one aspect (A79) of any one of A75-A77, the antibody or antigen-binding fragment thereof that specifically binds to human PILRA and the antagonist of the inhibitory immune checkpoint molecule are administered sequentially.

In one aspect (A80), provided herein is a method of treating a disease or condition in which myeloid cells are dysfunctional or deficient in a patient, the method comprising administering to the patient a therapeutically effective amount of the antibody or antigen binding fragment thereof of any one of A1-A50 and A60 or the pharmaceutical composition of A61, optionally further administering an antagonist of PD-L1.

In one aspect (A81), provided herein is a method of treating a neurodegenerative disease in a patient, the method comprising administering to the patient a therapeutically effective amount of the antibody or antigen binding fragment thereof of any one of A1-A50 and A60 or the pharmaceutical composition of A61, optionally further comprising administering an antagonist of PD-L1. In one aspect (A82) of A81, the neurodegenerative disease is selected from the group consisting of: Alzheimer's disease, dementia, frontotemporal dementia (FTD), vascular dementia, mild cognitive impairment, Parkinson's disease, amyotrophic lateral sclerosis (ALS), Huntington's disease, Tauopathy disease, multiple sclerosis (MS), immune-mediated neuropathies (such as neuropathic pain), Nasu-Hakola disease, pediatric-onset leukoencephalopathy, adult-onset leukoencephalopathy with axonal spheroids and pigmented glia (ALSP), and limbic-predominant age-related TDP43 encephalopathy (LATE). In one aspect (A83) of A81, the neurodegenerative disease is Alzheimer's disease, optionally wherein the patient carries the G78 variant of PILRA.

In one aspect (A84) of A83, the method increases MCP-1, IL-8, IFNg, TNFa, IL-6, and/or MIP1b in the patient.

In one aspect (A85), provided herein is a method of increasing MCP-1 in a monocyte comprising contacting the monocyte with the antibody or antigen-binding fragment thereof of any one of A1-A50 and A60 or the pharmaceutical composition of A61, optionally further comprising contacting an antagonist of PD-L1 with the monocyte. In one aspect (A86) of A85, the monocyte is in vitro. In one aspect (A87) of A85, the monocyte is in a subject. In one aspect (A88) of A87, the subject is a human subject.

In one aspect (A89) of any one of A85-A88, the contacting comprises administering the antibody, antigen-binding fragment thereof, or pharmaceutical composition to a subject.

In one aspect (A90), is a method of increasing IL-8 in a monocyte comprising contacting the monocyte with the antibody or antigen-binding fragment thereof of any one of A1-A50 and A60 or the pharmaceutical composition of A61, optionally further contacting an antagonist of PD-L1 with the monocyte. In one aspect (A91) of A90, the monocyte is in vitro. In one aspect (A92) of A90, the monocyte is in a subject. In one aspect (A93) of A92, the subject is a human subject. In one aspect (A94) of any one of A90-A93, the contacting comprises administering the antibody, antigen-binding fragment thereof, or pharmaceutical composition to a subject.

In one aspect (A95), provided herein is a method of increasing IFNg in a macrophage comprising contacting the macrophage with the antibody or antigen-binding fragment thereof of any one of A1-A50 and A60 or the pharmaceutical composition of A61, optionally further contacting an antagonist of PD-L1 with the macrophage. In one aspect (A96) of A95, the macrophage is in vitro. In one aspect (A97) of A95, the macrophage is in a subject. In one aspect (A98) of A96, the subject is a human subject.

In one aspect (A99) of any one of A95-A98, the contacting comprises administering the antibody, antigen-binding fragment thereof, or pharmaceutical composition to a subject.

In one aspect (A100), provided herein is a method of increasing TNFa in a macrophage comprising contacting the macrophage with the antibody or antigen-binding fragment thereof of any one of A1-A50 and A60 or the pharmaceutical composition of A61, optionally further contacting an antagonist of PD-L1 with the macrophage. In one aspect (A101) of A100, the macrophage is in vitro. In one aspect (A102) of A100, the macrophage is in a subject. In one aspect (A103) of A102, the subject is a human subject. In one aspect (A104) of any one of A100-A103, the contacting comprises administering the antibody, antigen-binding fragment thereof, or pharmaceutical composition to a subject.

In one aspect (A105), provided herein is a method of increasing IL-2 in a macrophage comprising contacting the macrophage with the antibody or antigen-binding fragment thereof of any one of A1-A50 and A60 or the pharmaceutical composition of A61, optionally further contacting an antagonist of PD-L1 with the macrophage. In one aspect (A106) of A105, the macrophage is in vitro. In one aspect (A107) of A105, the macrophage is in a subject. In one aspect (A108) of A107, the subject is a human subject. In one aspect (A109) of any one of A105-A108, the contacting comprises administering the antibody, antigen-binding fragment thereof, or pharmaceutical composition to a subject.

In one aspect (A110), provided herein is a method of increasing IL-6 in a macrophage comprising contacting the macrophage with the antibody or antigen-binding fragment thereof of any one of A1-A50 and A60 or the pharmaceutical composition of A61, optionally further contacting an antagonist of PD-L1 with the macrophage. In one aspect (A111) of A110, the macrophage is in vitro. In one aspect (A112) of A110, the macrophage is in a subject. In one aspect (A113) of A112, the subject is a human subject. In one aspect (A114) of any one of A110-A113, the contacting comprises administering the antibody, antigen-binding fragment thereof, or pharmaceutical composition to a subject.

In one aspect (A115) of any one of A85-A114, the antagonist of PD-L1 is an anti-PD-L1 antibody or antigen-binding fragment thereof, optionally wherein the anti-PD-L1 antibody or antigen-binding fragment thereof is selected from the group consisting of atezolizumab, durvalumab (MEDI4736), BMS-936559, MSB0010718C and rHigM12B7.

In one aspect (A116), provided herein is a method of activating the innate immune system in a patient, the method comprising administering to the patient an effective amount of the antibody or antigen binding fragment thereof of any one of A1-A50 and A60 or the pharmaceutical composition of A61.

In one aspect (A117), provided herein is a method for detecting PILRA in a sample comprising contacting the sample with the antibody or antigen-binding fragment thereof of any one of A1-A50 and A60 or the pharmaceutical composition of A61. In one aspect (A118) of A117, the sample is obtained from a human subject, optionally wherein the sample is a cancer sample.

In one aspect (A119), provided herein is the antibody or antigen-binding fragment thereof of any one of A1-A50 and A60 or the pharmaceutical composition of A61 for use in the method of any one of A62-A116.

In one aspect (A120), provided herein is the antibody or antigen-binding fragment thereof of any one of A1-A50 and A60 or the pharmaceutical composition of A61 for the manufacture of a medicament for use in the method of any one of A62-A85, A87-A90, A92-A95, A97-A100, A102-A105, A107-A110, and A112-A116.

In one aspect (A121), provided herein is an use of the antibody or or antigen-binding fragment thereof of any one of A1-A50 and A60 or the pharmaceutical composition of A61 in the method of any one of A62-A116.

### 6. BRIEF DESCRIPTION OF THE FIGURES

FIG. 1A shows a dose response curve of the tested anti-PILRA antibodies binding to PILRA on human monocytes as compared to an isotype control. (See Example 6.)

FIG. 1B shows a dose response curve of the tested anti-PILRA antibodies binding to PILRA on human monocytes as compared to an isotype control. (See Example 6.)

FIG. 1C shows the ability of the tested anti-PILRA antibodies (1 μg/mL) to bind to PILRA on human monocytes as compared to an isotype control. (See Example 6.)

FIG. 2 shows a dose response curve of the tested anti-PILRA antibodies binding to cynomolgus monocytes as compared to an isotype control. (See Example 6.)

FIG. 3 shows a dose response curve of the tested anti-PILRA antibodies to inhibit binding of NPDC1 to human PILRA molecules on the cell. (See Example 7.)

FIG. 4A shows a dose response curve of the tested anti-PILRA antibodies binding to cells overexpressing human PILRA. (See Example 8.)

FIG. 4B shows a dose response curve of the tested anti-PILRA antibodies binding to cells overexpressing cynomolgus PILRA. (See Example 8.)

FIG. 4C shows a dose response curve of the tested anti-PILRA antibodies binding to cells overexpressing human PILRB. (See Example 8.)

FIG. 5A shows a dose response curve of the tested anti-PILRA antibodies to downregulate PILRA on human monocytes. (See Example 9.)

FIG. 5B shows the ability of the tested anti-PILRA antibodies (1 μg/mL) to downregulate PILRA on human monocytes. (See Example 9.)

FIG. 6A shows a dose response curve of the tested anti-PILRA antibodies to induce MCP-1 on human monocytes. (See Example 10.)

FIG. 6B shows the ability of the tested anti-PILRA antibodies (1 μg/mL) to induce MCP-1 on human monocytes. (See Example 10.)

FIG. 6C shows the ability of the tested anti-PILRA antibodies (1 μg/mL) to induce MCP-1 on cynomolgus monocytes. (See Example 10.)

FIG. 7A shows induction of IFNg in an allogeneic mixed lymphocyte co-culture (human macrophage paired with CD3+T cells) when treated with the tested anti-PILRA antibodies or the anti-PILRA antibodies in combination with anti-PD-L1 treatment. (See Example 11.)

FIG. 7B shows induction of TNFa in an allogeneic mixed lymphocyte co-culture (human macrophage paired with CD3+T cells) when treated with the tested anti-PILRA antibodies or the anti-PILRA antibodies in combination with anti-PD-L1 treatment. (See Example 11.)

FIG. 7C shows induction of IL-2 in an allogeneic mixed lymphocyte co-culture (human macrophage paired with CD3+T cells) when treated with the tested anti-PILRA antibodies or the anti-PILRA antibodies in combination with anti-PD-L1 treatment. (See Example 11.)

FIG. 7D shows induction of IL-6 in an allogeneic mixed lymphocyte co-culture (human macrophage paired with CD3+T cells) when treated with the tested anti-PILRA antibodies or the anti-PILRA antibodies in combination with anti-PD-L1 treatment. (See Example 11.)

### 7. DETAILED DESCRIPTION

Provided herein are antibodies (e.g., monoclonal antibodies) and antigen-binding fragments thereof that specifically bind to PILRA, e.g., human PILRA. Anti-human PILRA antibodies and antigen-binding fragments thereof can, for example, block binding of human PILRA to ligand and/or downregulate cell surface human PILRA. Anti-human PILRA antibodies and antigen-binding fragments thereof can, for example, block binding of soluble PILRA to T cells. Exemplary anti-human PILRA antibodies are provided herein that demonstrate these activities. Blocking binding of human PILRA to ligand and/or downregulating cell surface human PILRA reduces inhibitory signaling by PILRA, resulting in activation and differentiation of myeloid cells. These activities may promote anti-tumor immunity and counteract the mechanisms of neurodegenerative diseases, such as Alzheimer's disease and other diseases associated with dysfunctional microglia.

Also provided are isolated nucleic acids (polynucleotides), such as complementary DNA (cDNA), encoding such antibodies and antigen-binding fragments thereof. Further provided are vectors (e.g., expression vectors) and cells (e.g., host cells) comprising nucleic acids (polynucleotides) encoding such antibodies and antigen-binding fragments thereof. Also provided are methods of making such antibodies and antigen-binding fragments thereof.

In other aspects, provided herein are methods for using such antibodies, for example, to modulate PILRA activity. PILRA activity can be modulated, for example, by altering the binding of PILRA to one or more of its ligands. In some aspects, anti-human PILRA antibodies provided herein are used to block the binding of human PILRA to ligand and/or to downregulate cell surface human PILRA.

In further aspects, anti-human PILRA antibodies provided herein are used to activate myeloid cells, such as macrophages, monocytes, dendritic cells, neutrophils, and microglia, in vitro or in vivo. In further aspects, anti-human PILRA antibodies provided herein are used to treat diseases in which myeloid cells are dysfunctional or deficient, e.g., diseases in which myeloid cell activation, myeloid cell differentiation, or activation of the innate immune system is desired. In some aspects, such diseases include, but are not limited to cancer and neurodegenerative diseases such as Alzheimer's disease. In particular, the R78 variant of PILRA, which is reported to reduce binding of PILRA to several of its ligands, is associated with reduced risk of Alzheimer's disease. Therefore, in some aspects, anti-human PILRA antibodies that reduce PILRA function (e.g., by blocking the binding of PILRA to ligand and/or downregulating cell surface human PILRA) would be useful for treating Alzheimer's disease. Related compositions (e.g., pharmaceutical compositions), kits, and methods are also provided.

### 7.1 Terminology

As used herein, the term “PILRA” refers to mammalian PILRA polypeptides including, but not limited to, native PILRA polypeptides and isoforms of PILRA polypeptides. “PILRA” encompasses full-length, unprocessed PILRA polypeptides as well as forms of PILRA polypeptides that result from processing within the cell. As used herein, the term “human PILRA” refers to a polypeptide comprising the amino acid sequence of SEQ ID NO:1; naturally occurring variants of SEQ ID NO:1, including but not limited to variants thereof in which either G or R is present at position 78 of SEQ ID NO: 1; and processed forms of SEQ ID NO:1, including but not limited to SEQ ID NO: 1 lacking its signal peptide, e.g., from amino acids 1-19 of SEQ ID NO:1. A “PILRA polynucleotide,” “PILRA nucleotide,” or “PILRA nucleic acid” refers to a polynucleotide encoding any PILRA, including those described above.

The term “antibody” means an immunoglobulin molecule that recognizes and specifically binds to a target, such as a protein, polypeptide, peptide, carbohydrate, polynucleotide, lipid, or combinations of the foregoing (e.g., a glycoprotein), through at least one antigen recognition site within the variable region of the immunoglobulin molecule. As used herein, the term “antibody” encompasses polyclonal antibodies, monoclonal antibodies, chimeric antibodies, humanized antibodies, human antibodies, and any other immunoglobulin molecule so long as the antibodies exhibit the desired biological activity. An antibody can be of any the five major classes of immunoglobulins: IgA, IgD, IgE, IgG, and IgM, or subclasses (isotypes) thereof (e.g. IgG1, IgG2, IgG3, IgG4, IgAQ1 and IgA2), based on the identity of their heavy-chain constant domains referred to as alpha, delta, epsilon, gamma, and mu, respectively. The different classes of immunoglobulins have different and well known subunit structures and three-dimensional configurations. Antibodies can be naked, part of a fusion protein, or conjugated to other molecules such as toxins, radioisotopes, etc.

The term “antibody fragment” refers to a portion of an antibody. An “antigen-binding fragment,” “antigen-binding domain,” or “antigen-binding region,” refers to a portion of an antibody that binds to an antigen. An antigen-binding fragment can contain the antigenic determining regions of an antibody (e.g., the complementarity determining regions (CDR)). Examples of antigen-binding fragments of antibodies include, but are not limited to Fab, Fab′, F(ab′)₂, and Fv fragments, linear antibodies, and single chain antibodies. An antigen-binding fragment of an antibody can be derived from any animal species, such as rodents (e.g., mouse, rat, or hamster) and humans or can be artificially produced.

The terms “anti-PILRA antibody,” “PILRA antibody” and “antibody that binds to PILRA” refer to an antibody that is capable of binding PILRA with sufficient affinity such that the antibody is useful as a diagnostic, a therapeutic, and/or as a modulator of PILRA activity. The extent of binding of an anti-PILRA antibody to an unrelated, non-PILRA protein can be less than about 10% of the binding of the antibody to PILRA as measured, e.g., by a radioimmunoassay (RIA). An anti-PILRA antibody can bind exclusively to PILRA and not to PILRB, or an anti-PILRA antibody can bind to PILRA and to PILRB.

A “monoclonal” antibody or antigen-binding fragment thereof refers to a homogeneous antibody or antigen-binding fragment population involved in the highly specific recognition and binding of a single antigenic determinant, or epitope. This is in contrast to polyclonal antibodies that typically include different antibodies directed against different antigenic determinants. The term “monoclonal” antibody or antigen-binding fragment thereof encompasses both intact and full-length monoclonal antibodies as well as antibody fragments (such as Fab, Fab′, F(ab′)2, Fv), single chain (scFv) mutants, fusion proteins comprising an antibody portion, and any other modified immunoglobulin molecule comprising an antigen recognition site. Furthermore, a “monoclonal” antibody or antigen-binding fragment thereof refers to such antibodies and antigen-binding fragments thereof made in any number of manners including but not limited to by hybridoma, phage selection, recombinant expression, and transgenic animals.

As used herein, the terms “variable region” or “variable domain” are used interchangeably and are common in the art. The variable region typically refers to a portion of an antibody, generally, a portion of a light or heavy chain, typically about the amino-terminal 110 to 120 amino acids or 110 to 125 amino acids in the mature heavy chain and about 90 to 115 amino acids in the mature light chain, which differ extensively in sequence among antibodies and are used in the binding and specificity of a particular antibody for its particular antigen. The variability in sequence is concentrated in those regions called complementarity determining regions (CDRs) while the more highly conserved regions in the variable domain are called framework regions (FR). Without wishing to be bound by any particular mechanism or theory, it is believed that the CDRs of the light and heavy chains are primarily responsible for the interaction and specificity of the antibody with antigen. In some aspects, the variable region is a human variable region. In some aspects, the variable region comprises rodent or murine CDRs and human framework regions (FRs). In some aspects, the variable region is a primate (e.g., non-human primate) variable region. In some aspects, the variable region comprises rodent or murine CDRs and primate (e.g., non-human primate) framework regions (FRs).

The terms “VL” and “VL domain” are used interchangeably to refer to the light chain variable region of an antibody.

The terms “VH” and “VH domain” are used interchangeably to refer to the heavy chain variable region of an antibody.

The term “Kabat numbering” and like terms are recognized in the art and refer to a system of numbering amino acid residues in the heavy and light chain variable regions of an antibody or an antigen-binding fragment thereof. In certain aspects, CDRs can be determined according to the Kabat numbering system (see, e.g., Kabat E A & Wu T T (1971) Ann NY Acad Sci 190:382-391 and Kabat E A et al., (1991) Sequences of Proteins of Immunological Interest, Fifth Edition, U.S. Department of Health and Human Services, NIH Publication No. 91-3242). Using the Kabat numbering system, CDRs within an antibody heavy chain molecule are typically present at amino acid positions 31 to 35, which optionally can include one or two additional amino acids, following 35 (referred to in the Kabat numbering scheme as 35A and 35B) (CDR1), amino acid positions 50 to 65 (CDR2), and amino acid positions 95 to 102 (CDR3). Using the Kabat numbering system, CDRs within an antibody light chain molecule are typically present at amino acid positions 24 to 34 (CDR1), amino acid positions 50 to 56 (CDR2), and amino acid positions 89 to 97 (CDR3). In some aspects, the CDRs of the antibodies described herein have been determined according to the Kabat numbering scheme.

Chothia refers instead to the location of the structural loops (Chothia and Lesk, J. Mol. Biol. 196:901-917 (1987)). The end of the Chothia CDR-H1 loop when numbered using the Kabat numbering convention varies between H32 and H34 depending on the length of the loop (this is because the Kabat numbering scheme places the insertions at H35A and H35B; if neither 35A nor 35B is present, the loop ends at 32; if only 35A is present, the loop ends at 33; if both 35A and 35B are present, the loop ends at 34). The AbM hypervariable regions represent a compromise between the Kabat CDRs and Chothia structural loops, and are used by Oxford Molecular's AbM antibody modeling software.

| Loop | Kabat | AbM | Chothia |
|---|---|---|---|
| L1 | L24-L34 | L24-L34 | L24-L34 |
| L2 | L50-L56 | L50-L56 | L50-L56 |
| L3 | L89-L97 | L89-L97 | L89-L97 |
| H1 | H31-H35B | H26-H35B | H26-H32 . . . 34 |

(Kabat Numbering)

| | | | |
|---|---|---|---|
| H1 | H31-H35 | H26-H35 | H26-H32 |

(Chothia Numbering)

| | | | |
|---|---|---|---|
| H2 | H50-H65 | H50-H58 | H52-H56 |
| H3 | H95-H102 | H95-H102 | H95-H102 |

As used herein, the term “constant region” or “constant domain” are interchangeable and have the meaning common in the art. The constant region is an antibody portion, e.g., a carboxyl terminal portion of a light and/or heavy chain which is not directly involved in binding of an antibody to antigen but which can exhibit various effector functions, such as interaction with the Fc receptor. The constant region of an immunoglobulin molecule generally has a more conserved amino acid sequence relative to an immunoglobulin variable domain. In certain aspects, an antibody or antigen-binding fragment comprises a constant region or portion thereof that is sufficient for antibody-dependent cell-mediated cytotoxicity (ADCC).

As used herein, the term “heavy chain” when used in reference to an antibody can refer to any distinct type, e.g., alpha (α), delta (δ), epsilon (ε), gamma (γ), and mu (μ), based on the amino acid sequence of the constant domain, which give rise to IgA, IgD, IgE, IgG, and IgM classes of antibodies, respectively, including subclasses of IgG, e.g., IgG1, IgG2, IgG3, and IgG4. Heavy chain amino acid sequences are well known in the art. In some aspects, the heavy chain is a human heavy chain.

As used herein, the term “light chain” when used in reference to an antibody can refer to any distinct type, e.g., kappa (κ) or lambda (λ) based on the amino acid sequence of the constant domains. Light chain amino acid sequences are well known in the art. In some aspects, the light chain is a human light chain.

The term “chimeric” antibodies or antigen-binding fragments thereof refers to antibodies or antigen-binding fragments thereof wherein the amino acid sequence is derived from two or more species. Typically, the variable region of both light and heavy chains corresponds to the variable region of antibodies or antigen-binding fragments thereof derived from one species of mammals (e.g. mouse, rat, rabbit, etc.) with the desired specificity, affinity, and capability while the constant regions are homologous to the sequences in antibodies or antigen-binding fragments thereof derived from another (usually human) to avoid eliciting an immune response in that species.

The term “humanized” antibody or antigen-binding fragment thereof refers to forms of non-human (e.g. murine) antibodies or antigen-binding fragments that are specific immunoglobulin chains, chimeric immunoglobulins, or fragments thereof that contain minimal non-human (e.g., murine) sequences. Typically, humanized antibodies or antigen-binding fragments thereof are human immunoglobulins in which residues from the complementarity determining regions (CDRs) are replaced by residues from the CDRs of a non-human species (e.g. mouse, rat, rabbit, hamster) that have the desired specificity, affinity, and capability (“CDR grafted”) (Jones et al., Nature 321:522-525 (1986); Riechmann et al., Nature 332:323-327 (1988); Verhoeyen et al., Science 239:1534-1536 (1988)). The humanized antibody or antigen-binding fragment thereof can be further modified by the substitution of additional residues either in the Fv framework region and/or within the replaced non-human residues to refine and optimize the specificity, affinity, and/or capability of the antibody or antigen-binding fragment thereof. In general, the humanized antibody or antigen-binding fragment thereof will comprise VH and VL that comprise substantially all of at least one, and typically two or three, of the CDR regions that correspond to the non-human immunoglobulin, whereas all or substantially all of the FR regions are those of a human immunoglobulin consensus sequence. The humanized antibody or antigen-binding fragment thereof can also comprise at least a portion of an immunoglobulin constant region or domain (Fc), typically that of a human immunoglobulin. Examples of methods used to generate humanized antibodies are described in U.S. Pat. No. 5,225,539; Roguska et al., Proc. Natl. Acad. Sci., USA, 91 (3): 969-973 (1994), and Roguska et al., Protein Eng. 9 (10): 895-904 (1996). In some aspects, a “humanized antibody” is a resurfaced antibody.

The term “human” antibody or antigen-binding fragment thereof means an antibody or antigen-binding fragment thereof having an amino acid sequence derived from a human immunoglobulin gene locus, where such antibody or antigen-binding fragment is made using any technique known in the art. This definition of a human antibody or antigen-binding fragment thereof includes intact or full-length antibodies and fragments thereof.

“Binding affinity” generally refers to the strength of the sum total of non-covalent interactions between a single binding site of a molecule (e.g., an antibody or antigen-binding fragment thereof) and its binding partner (e.g., an antigen). Unless indicated otherwise, as used herein, “binding affinity” refers to intrinsic binding affinity which reflects a 1:1 interaction between members of a binding pair (e.g., antibody or antigen-binding fragment thereof and antigen). The affinity of a molecule X for its partner Y can generally be represented by the dissociation constant (K_{D}). Affinity can be measured and/or expressed in a number of ways known in the art, including, but not limited to, equilibrium dissociation constant (K_{D}), and equilibrium association constant (K_{A}). The K_{D} is calculated from the quotient of k_{off}/kₒₙ, whereas K_{A} is calculated from the quotient of kₒₙ/k_{off}. kₒₙ refers to the association rate constant of, e.g., an antibody or antigen-binding fragment thereof to an antigen, and k_{off} refers to the dissociation rate constant of, e.g., an antibody or antigen-binding fragment thereof from an antigen. The kₒₙ and k_{off} can be determined by techniques known to one of ordinary skill in the art, such as BIAcore® or KinExA.

A antibody that is “blocking” or that “blocks” or that is “inhibitory” of that “inhibits” is an antibody that reduces or inhibits (partially or completely) binding of its target protein to one or more ligands when the antibody is bound to the target protein, and/or that reduces or inhibits (partially or completely) one or more activities or functions of the target protein when the antibody is bound to the target protein.

An antibody that “downregulates” its target protein reduces expression of the target protein on the cell surface.

As used herein, an “epitope” is a term in the art and refers to a localized region of an antigen to which an antibody or antigen-binding fragment thereof can specifically bind. An epitope can be, for example, contiguous amino acids of a polypeptide (linear or contiguous epitope) or an epitope can, for example, come together from two or more non-contiguous regions of a polypeptide or polypeptides (conformational, non-linear, discontinuous, or non-contiguous epitope). In some aspects, the epitope to which an antibody or antigen-binding fragment thereof binds can be determined by, e.g., NMR spectroscopy, X-ray diffraction crystallography studies, ELISA assays, hydrogen/deuterium exchange coupled with mass spectrometry (e.g., liquid chromatography electrospray mass spectrometry), array-based oligo-peptide scanning assays, and/or mutagenesis mapping (e.g., alanine scanning or other site-directed mutagenesis mapping). For X-ray crystallography, crystallization may be accomplished using any of the known methods in the art (e.g., Giegé R et al., (1994) Acta Crystallogr D Biol Crystallogr 50 (Pt 4): 339-350; McPherson A (1990) Eur J Biochem 189:1-23; Chayen N E (1997) Structure 5:1269-1274; McPherson A (1976) J Biol Chem 251:6300-6303). Crystals of an antibody or antigen-binding fragment thereof and its antigen can be studied using well known X-ray diffraction techniques and can be refined using computer software such as X-PLOR (Yale University, 1992, distributed by Molecular Simulations, Inc.; see, e.g., Meth Enzymol (1985) volumes 114 & 115, eds Wyckoff H W et al.,; U.S. 2004/0014194), and BUSTER (Bricogne G (1993) Acta Crystallogr D Biol Crystallogr 49 (Pt 1): 37-60; Bricogne G (1997) Meth Enzymol 276A: 361-423, ed Carter C W; Roversi P et al., (2000) Acta Crystallogr D Biol Crystallogr 56 (Pt 10): 1316-1323). Mutagenesis mapping studies can be accomplished using any method known to one of skill in the art. See, e.g., Champe M et al., (1995) J Biol Chem 270:1388-1394 and Cunningham B C & Wells J A (1989) Science 244:1081-1085 for a description of mutagenesis techniques, including alanine scanning mutagenesis techniques.

A PILRA antibody that “binds to the same epitope” as a reference PILRA antibody refers to an antibody that contacts the same PILRA amino acid residues as the reference PILRA antibody. The ability of a PILRA antibody to bind to the same epitope as a reference PILRA antibody is determined using peptide scanning mutagenesis or high throughput alanine scanning mutagenesis (see Davidson and Doranz, 2014 Immunology 143, 13-20). In the latter methodology, a comprehensive mutation library of PILRA, or a portion thereof (e.g., the extracellular domain), can be generated by mutating each individual amino acid residue to alanine (or if the amino acid residue is alanine, then to another residue such as serine) and testing each mutant for binding to an anti-PILRA antibody or antigen binding fragment thereof. Amino acids that are required for binding, and therefore are epitope residues, are identified by loss of immunoreactivity.

As used herein, the terms “immunospecifically binds,” “immunospecifically recognizes,” “specifically binds,” and “specifically recognizes” are analogous terms in the context of antibodies or antigen-binding fragments thereof. These terms indicate that the antibody or antigen-binding fragment thereof binds to an epitope via its antigen-binding domain and that the binding entails complementarity between the antigen binding domain and the epitope. Accordingly, an antibody that “specifically binds” to human PILRA (e.g., SEQ ID NO:1) may also bind to PILRA from other species (e.g., cynomolgus monkey PILRA), PILRA proteins produced from other human alleles, and/or PILRB proteins, but the extent of binding to an un-related, non-PILRA protein (e.g., other immunomodulatory proteins containing ITIM domains) is less than about 10% of the binding of the antibody to PILRA as measured, e.g., by a radioimmunoassay (RIA).

An antibody is said to “competitively inhibit” binding of a reference antibody to a given epitope if it preferentially binds to that epitope or an overlapping epitope such that it blocks, to some degree, binding of the reference antibody to the epitope. Competitive inhibition may be determined by any method known in the art, for example, competition ELISA assays. An antibody may be said to competitively inhibit binding of the reference antibody to a given epitope by at least 90%, at least 80%, at least 70%, at least 60%, or at least 50%.

A polypeptide, antibody, polynucleotide, vector, cell, or composition which is “isolated” is a polypeptide, antibody, polynucleotide, vector, cell, or composition which is in a form not found in nature. Isolated polypeptides, antibodies, polynucleotides, vectors, cells or compositions include those which have been purified to a degree that they are no longer in a form in which they are found in nature. In some aspects, an antibody, polynucleotide, vector, cell, or composition which is isolated is substantially pure. As used herein, “substantially pure” refers to material which is at least 50% pure (i.e., free from contaminants), at least 90% pure, at least 95% pure, at least 98% pure, or at least 99% pure.

The terms “polypeptide,” “peptide,” and “protein” are used interchangeably herein to refer to polymers of amino acids of any length. The polymer can be linear or branched, it can comprise modified amino acids, and it can be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. It is understood that, because the polypeptides of this disclosure are based upon antibodies, in some aspects, the polypeptides can occur as single chains or associated chains.

As used herein, the term “host cell” can be any type of cell, e.g., a primary cell, a cell in culture, or a cell from a cell line. In some aspects, the term “host cell” refers to a cell transfected with a nucleic acid molecule and the progeny or potential progeny of such a cell. Progeny of such a cell may not be identical to the parent cell transfected with the nucleic acid molecule, e.g., due to mutations or environmental influences that may occur in succeeding generations or integration of the nucleic acid molecule into the host cell genome.

The term “pharmaceutical formulation” refers to a preparation which is in such form as to permit the biological activity of the active ingredient to be effective, and which contains no additional components which are unacceptably toxic to a subject to which the formulation would be administered. The formulation can be sterile.

The terms “administer,” “administering,” “administration,” and the like, as used herein, refer to methods that may be used to deliver a drug, e.g., an anti-human PILRA antibody or antigen-binding fragment thereof, to the desired site of biological action. Administration techniques that can be employed with the agents and methods described herein are found in e.g., Goodman and Gilman, The Pharmacological Basis of Therapeutics, current edition, Pergamon; and Remington's, Pharmaceutical Sciences, current edition, Mack Publishing Co., Easton, Pa.

As used herein, the terms “subject” and “patient” are used interchangeably. The subject can be a mammal such as a non-human animal (e.g., cow, pig, horse, cat, dog, rat, mouse, monkey or other primate, etc.). In some aspects, the subject is a cynomolgus monkey. In some aspects, the subject is a human.

The term “therapeutically effective amount” refers to an amount of a drug, e.g., an anti-human PILRA antibody or antigen-binding fragment thereof, effective to treat a disease or condition in a subject. In the case of cancer, the therapeutically effective amount of the drug can reduce the number of cancer cells; reduce the tumor size or burden; inhibit (i.e., slow to some extent and in some aspects, stop) cancer cell infiltration into peripheral organs; inhibit (i.e., slow to some extent and in some aspects, stop) tumor metastasis; inhibit, to some extent, tumor growth; relieve to some extent one or more of the symptoms associated with the cancer; and/or result in a favorable response such as increased progression-free survival (PFS), disease-free survival (DFS), or overall survival (OS), complete response (CR), partial response (PR), or, in some cases, stable disease (SD), a decrease in progressive disease (PD), a reduced time to progression (TTP), or any combination thereof. To the extent the drug can prevent growth and/or kill existing cancer cells, it can be cytostatic and/or cytotoxic.

Terms such as “treating” or “treatment” or “to treat” or “alleviating” or “to alleviate” refer to therapeutic measures that cure, slow down, lessen symptoms of, and/or halt progression of a diagnosed pathologic condition or disorder. Thus, those in need of treatment include those already diagnosed with or suspected of having the disorder. In some aspects, a subject is successfully “treated” for cancer according to the methods provided herein if the patient shows one or more of the following: a reduction in the number of or complete absence of cancer cells; a reduction in the tumor size; inhibition of or an absence of cancer cell infiltration into peripheral organs including, for example, the spread of cancer into soft tissue and bone; inhibition of or an absence of tumor metastasis; inhibition or an absence of tumor growth; relief of one or more symptoms associated with the specific cancer; reduced morbidity and mortality; improvement in quality of life; reduction in tumorigenicity, tumorigenic frequency, or tumorigenic capacity, of a tumor; reduction in the number or frequency of cancer stem cells in a tumor; differentiation of tumorigenic cells to a non-tumorigenic state; increased progression-free survival (PFS), disease-free survival (DFS), or overall survival (OS), complete response (CR), partial response (PR), stable disease (SD), a decrease in progressive disease (PD), a reduced time to progression (TTP), or any combination thereof.

The terms “cancer” and “cancerous” refer to or describe the physiological condition in mammals in which a population of cells are characterized by unregulated cell growth. Such cancers can include solid tumors, e.g., solid tumors in which myeloid cells (monocytes, macrophages, dendritic cells, granulocytes, neutrophils, microglia or other innate immune cells) have infiltrated the tumor microenvironment. Examples of such cancers include, but are not limited to, glioblastoma, head and neck cancer, kidney cancer (e.g., kidney clear cell cancer), pancreatic cancer, and breast cancer. The cancer can be a “PILRA-positive cancer.” This term refers to a cancer comprising cells (e.g., myeloid cells that have infiltrated the cancer) that express PILRA mRNA or protein. The cancer can be a cancer with “increased PILRA” mRNA or protein This refers to a cancer that has more PILRA (e.g., on myeloid cells that have infiltrated the cancer) than a healthy version of the same tissue.

As used in the present disclosure and claims, the singular forms “a,” “an,” and “the” include plural forms unless the context clearly dictates otherwise.

It is understood that wherever aspects are described herein with the language “comprising,” otherwise analogous aspects described in terms of “consisting of” and/or “consisting essentially of” are also provided. In this disclosure, “comprises,” “comprising,” “containing” and “having” and the like can mean “includes,” “including,” and the like; “consisting essentially of” or “consists essentially of” are open-ended, allowing for the presence of more than that which is recited so long as basic or novel characteristics of that which is recited is not changed by the presence of more than that which is recited, but excludes prior art aspects.

Unless specifically stated or obvious from context, as used herein, the term “or” is understood to be inclusive. The term “and/or” as used in a phrase such as “A and/or B” herein is intended to include both “A and B,” “A or B,” “A,” and “B.” Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

As used herein, the terms “about” and “approximately,” when used to modify a numeric value or numeric range, indicate that deviations of up to 10% above and down to 10% below the value or range remain within the intended meaning of the recited value or range. It is understood that wherever aspects are described herein with the language “about” or “approximately” a numeric value or range, otherwise analogous aspects referring to the specific numeric value or range are also provided.

Any compositions or methods provided herein can be combined with one or more of any of the other compositions and methods provided herein.

### 7.2 Antibodies

In one aspect, provided herein are antibodies (e.g., monoclonal antibodies, such as chimeric, humanized, or human antibodies) and antigen-binding fragments thereof which specifically bind to PILRA, such as human, murine or cynomolgus monkey PILRA. In a specific aspect, provided herein are antibodies (e.g., monoclonal antibodies, such as chimeric, humanized, or human antibodies) and antigen-binding fragments thereof which specifically bind to human PILRA. The amino acid sequences of human, cynomolgus monkey, and murine PILRA are known in the art and also provided herein as represented by SEQ ID NOs: 1-3 respectively.

Human PILRA:
(SEQ ID NO: 1)
MGRPLLLPLLPLLLPPAFLQPSGSTGSGPSYLYGVTQPKHLSASM
GGSVEIPFSFYYPWELATAPDVRISWRRGHFHRQSFYSTRPPSIH
KDYVNRLFLNWTEGQKSGFLRISNLQKQDQSVYFCRVELDTRSSG
RQQWQSIEGTKLSITQAVTTTTQRPSSMTTTWRLSSTTTTTGLRV
TQGKRRSDSWHISLETAVGVAVAVTVLGIMILGLICLLRWRRRKG
QQRTKATTPAREPFQNTEEPYENIRNEGQNTDPKLNPKDDGIVYA
SLALSSSTSPRAPPSHRPLKSPQNETLYSVLKA

In some aspects it is contemplated that the above human PILRA sequence lacks its signal sequence. For example, a human PILRA sequence can comprise amino acids 20-303 of SEQ ID NO:1. The above human PILRA sequence (SEQ ID NO:1) represents a variant sequence in which arginine (R) is present at position 78, and is encoded by an allele associated with protection from Alzheimer's disease. In some aspects, a variant PILRA sequence is contemplated in which position 78 is occupied by a glycine (G). Other features of human PILRA, as shown in SEQ ID NO:1, include an extracellular domain (ECD) from about amino acid 20-197, with an IgV domain from about amino acid 32-150, and ITIM motifs from about amino acid 267-272 and 296-301.

Cynomolgus monkey PILRA
(SeqA - XP_015302640.1 GenBank):
(SEQ ID NO: 2)
MGRPLLLPLLLPLLPLLLPPAFLQPGGSAGSGPSGPYGVTQRKHL
SAPMGGSVEIPFSFYHPWELAAAPNMKISWRRGNFHGEFFYRTRP
AFIHEDYSNRLLLNWTEGQDRGLLRIWNLRKEDQSVYFCRVELDT
RRSGRQRWQSIEGTKLTITQAVTTTTQRPSSMTTTRRPSSATTTA
GLRVTQGKRHSDSWHLSLKTAVGVTVAVAVLGIMILGLICLLRWR
RRKGQQRTKATTPAKEPFQNTEEPYENIRNEGQNTDPKPNPKDDG
IVYASLALSSSTSPRVPPSHHPLKSPQNETLYSVLKV

In some aspects it is contemplated that the above cynomolgus monkey PILRA sequence lacks its signal sequence. For example, a cynomolgus monkey PILRA sequence can comprise amino acids 24-307 of SEQ ID NO:2.

Cynomolgus monkey PILRA
(SeqB - XP_028701418.1 GenBank):
(SEQ ID NO: 120)
MGRPLLLPLLPLLLPPAFLQPGGSAGSGPSGPYGVTQRKHLSAPM
GGSVEIPFSFYHPWELAAAPNMKISWRRGNFHGEFFYRTRPAFIH
EDYSNRLLLNWTEGQDRGLLRIWNLRKEDQSVYFCRVELDTRRSG
RQRWQSIEGTKLTITQAVTTTTRRPSSATTTAGLRVTQGKRHSDS
WHLSLKTAVGVTVAVAVLGIMILGLICLLRWRRRKGQQRTKATTP
AKEPFQNTEEPYENIRNEGQNTDPKPNPKDDGIVYASLALSSSTS
PRVPPSHHPLKSPQNETLYSVLKV
Murine PILRA
(SEQ ID NO: 3)
MALLISLPGGTPAMAQILLLLSSACLHAGNSERSNRKNGFGVNQP
ESCSGVQGGSIDIPFSFYFPWKLAKDPQMSIAWRWKDFHGEFIYN
SSLPFIHEHFKGRLILNWTQGQTSGVLRILNLKESDQTRYFGRVF
LQTTEGIQFWQSIPGTQLNVTNATCTPTTLPSTTAATSAHTQNDI
TEVKSANIGGLDLQTTVGLATAAAVFLVGVLGLIVFLWWKRRRQG
QKTKAEIPAREPLETSEKHESVGHEGQCMDPKENPKDNNIVYASI
SLSSPTSPGTAPNLPVHGNPQEETVYSIVKAK.

In some aspects it is contemplated that the above murine PILRA sequence lacks its signal sequence. For example, a murine PILRA sequence can comprise amino acids 32-302 of SEQ ID NO:3.

In some aspects, antibodies or antigen-binding fragments thereof described herein bind to human PILRA and cynomolgus PILRA, block binding of soluble human PILRA to T-cells, and block binding of human PILRA to one or more of its ligands (e.g., NPDC1). In some aspects, antibodies or antigen-binding fragments thereof described herein block binding of soluble human PILRA to T-cells. In some aspects, the antibodies or antigen-binding fragments thereof described herein can downregulate PILRA.

In some aspects, an antibody or antigen-binding fragment thereof described herein binds to human PILRA (e.g., SEQ ID NO: 1 or amino acids 20-303 of SEQ ID NO: 1, or either of the foregoing sequences in which R or G is at position 78). In some aspects, an antibody or antigen-binding fragment thereof binds to human PILRA and cynomolgus monkey PILRA (e.g., SEQ ID NO: 2 or amino acids 24-307 of SEQ ID NO:2). In some aspects, an antibody or antigen-binding fragment thereof binds to human PILRA but does not bind to murine PILRA (e.g., SEQ ID NO:3 or amino acids 32-302 of SEQ ID NO:3). In some aspects, an antibody or antigen-binding fragment thereof binds to human PILRA (and optionally to cynomolgus monkey PILRA) and to human PILRB (e.g., SEQ ID NO:105, as shown below, or amino acids 20-227 of SEQ ID NO:105).

The sequence of human PILRB is provided
below as SEQ ID NO: 105.
(SEQ ID NO: 105)
MGRPLLLPLLLLLQPPAFLQPGGSTGSGPSYLYGVTQPKHLSASM
GGSVEIPFSFYYPWELAIVPNVRISWRRGHFHGQSFYSTRPPSIH
KDYVNRLFLNWTEGQESGFLRISNLRKEDQSVYFCRVELDTRRSG
RQQLQSIKGTKLTITQAVTTTTTWRPSSTTTIAGLRVTESKGHSE
SWHLSLDTAIRVALAVAVLKTVILGLLCLLLLWWRRRKGSRAPSS
DF

In some aspects it is contemplated that the above human PILRB sequence lacks its signal sequence. For example, a human PILRB sequence can comprise amino acids 20-227 of SEQ ID NO:105.

The sequence of cynomolgus monkey PILRB is
provided below as SEQ ID NO: 121.
(SEQ ID NO: 121)
MGRPLLLPLLPLLLPPAFLQPGGSAGSGPSGPYGVTQRKHLSAPM
GGSVEIPFSFYHPWELAAAPNMKISWRRGNFHGEFFYRTRPAFIH
EDYSNRLLLNWTEGQDRGLLRIWNLRKEDQSVYFCRVELDTRRSG
MQRLQSIEGTKLTITQAVMTTTTWRPSSATTTAGLRVTEGKGHSE
SWHLSLDTAISVALAVAVLKTVTLGLLCLLRWKRRKGQQQTKATT
PARVWRDGMCISPRGCAVRPACEYSTLAPHWQDTWRAS

In some aspects an antibody or antigen-binding fragment thereof described herein binds to the extracellular domain of human PILRA (amino acids 20-197 of SEQ ID NO:1).

In some aspects, an antibody or antigen-binding fragment thereof described herein binds to human PILRA and comprises the six CDRs of an antibody listed in Tables 1 and 2 (i.e., the three VH CDRs of the antibody listed in Table 1 and the three VL CDRs of the same antibody listed in Table 2).

TABLE 1
VH CDR Amino Acid Sequences ¹

| Anti- | VH CDR1 | VH CDR2 | VH CDR3 |
|---|---|---|---|
| body | (SEQ ID NO:) | (SEQ ID NO:) | (SEQ ID NO:) |
| X0 | NYDVN (SEQ ID | GIIPIFGTANYAQKFQG | GVGAGWFDP |
| (PA-29) | NO: 4) | (SEQ ID NO: 5) | (SEQ ID NO: 6) |
| X1 | HYDVN (SEQ ID | GIIPIFGTANYAQKFQG | AVGAGWFDT (SEQ ID |
| | NO: 10) | (SEQ ID NO: 5) | NO: 11) |
| X2 | HYDVN (SEQ ID | GIIPIFGTANYAQKFQG | AVGAGWFDP (SEQ ID |
| | NO: 10) | (SEQ ID NO: 5) | NO: 12) |
| X3 | NYDVN (SEQ ID | GIIPIFGTANYAQKFQG | GVGAGWFDP (SEQ ID |
| | NO: 4) | (SEQ ID NO: 5) | NO: 6) |
| X4 | NYDVN (SEQ ID | GIIPIFGTANYAQKFQG | GVGAGWFDP (SEQ ID |
| | NO: 4) | (SEQ ID NO: 5) | NO: 6) |
| X5 | NYDVN (SEQ ID | GIIPIFGTANYAQKFQG | GVGAGWFDP (SEQ ID |
| | NO: 4) | (SEQ ID NO: 5) | NO: 6) |
| X6 | NYDVN (SEQ ID | GIIPIFGTANYAQKFQG | GVGAGWFDP (SEQ ID |
| | NO: 4) | (SEQ ID NO: 5) | NO: 6) |
| X7 | NYDVN (SEQ ID | GIIPIFGTANYAQKFQG | GVGAGWFDP (SEQ ID |
| | NO: 4) | (SEQ ID NO: 5) | NO: 6) |
| X8 | NYDVN (SEQ ID | GIIPIFGTANYAQKFQG | GVGAGWFDP (SEQ ID |
| | NO: 4) | (SEQ ID NO: 5) | NO: 6) |
| X9 | NYDVN (SEQ ID | GIIPIFGTANYAQKFQG | GVGAGWFDP (SEQ ID |
| | NO: 4) | (SEQ ID NO: 5) | NO: 6) |
| X10 | NYDVN (SEQ ID | GIIPIFGTANYAQKFQG | GVGAGWFDP (SEQ ID |
| | NO: 4) | (SEQ ID NO: 5) | NO: 6) |
| X11 | NYDVN (SEQ ID | GIIPIFGTANYAQKFQG | GVGAGWFDP (SEQ ID |
| | NO: 4) | (SEQ ID NO: 5) | NO: 6) |
| X12 | HYDVN (SEQ ID | GIIPIFGTANYAQKFQG | AVGAGWFDT (SEQ ID |
| | NO: 10) | (SEQ ID NO: 5) | NO: 11) |
| X13 | HYDVN (SEQ ID | GIIPIFGTANYAQKFQG | AVGAGWFDP (SEQ ID |
| | NO: 10) | (SEQ ID NO: 5) | NO: 12) |
| X14 | HYDVN (SEQ ID | GIIPIFGTANYAQKFQG | AVGAGWFDT (SEQ ID |
| | NO: 10) | (SEQ ID NO: 5) | NO: 11) |
| X15 | HYDVN (SEQ ID | GIIPIFGTANYAQKFQG | AVGAGWFDT (SEQ ID |
| | NO: 10) | (SEQ ID NO: 5) | NO: 11) |
| X16 | HYDVN (SEQ ID | GIIPIFGTANYAQKFQG | AVGAGWFDT (SEQ ID |
| | NO: 10) | (SEQ ID NO: 5) | NO: 11) |
| X17 | HYDVN (SEQ ID | GIIPIFGTANYAQKFQG | AVGAGWFDT (SEQ ID |
| | NO: 10) | (SEQ ID NO: 5) | NO: 11) |
| X18 | HYDVN (SEQ ID | GIIPIFGTANYAQKFQG | AVGAGWFDT (SEQ ID |
| | NO: 10) | (SEQ ID NO: 5) | NO: 11) |
| X19 | HYDVN (SEQ ID | GIIPIFGTANYAQKFQG | AVGAGWFDP (SEQ ID |
| | NO: 10) | (SEQ ID NO: 5) | NO: 12) |
| X20 | HYDVN (SEQ ID | GIIPIFGTANYAQKFQG | AVGAGWFDP (SEQ ID |
| | NO: 10) | (SEQ ID NO: 5) | NO: 12) |
| X21 | HYDVN (SEQ ID | GIIPIFGTANYAQKFQG | GVGAGWFDP (SEQ ID |
| | NO: 10) | (SEQ ID NO: 5) | NO: 6) |
| X22 | HYDVN (SEQ ID | GIIPIFGTANYAQKFQG | GVGAGWFDP (SEQ ID |
| | NO: 10) | (SEQ ID NO: 5) | NO: 6) |
| X23 | HYDVN (SEQ ID | GIIPIFGTANYAQKFQG | GVGAGWFDP (SEQ ID |
| | NO: 10) | (SEQ ID NO: 5) | NO: 6) |
| X24 | HYDVN (SEQ ID | GIIPIFGTANYAQKFQG | GVGAGWFDP (SEQ ID |
| | NO: 10) | (SEQ ID NO: 5) | NO: 6) |
| X25 | NYDVN (SEQ ID | GIIPIFGTANYAQKFQG | AVGAGWFDP (SEQ ID |
| | NO: 4) | (SEQ ID NO: 5) | NO: 12) |
| X26 | NYDVN (SEQ ID | GIIPIFGTANYAQKFQG | AVGAGWFDP (SEQ ID |
| | NO: 4) | (SEQ ID NO: 5) | NO: 12) |
| X27 | NYDVN (SEQ ID | GIIPIFGTANYAQKFQG | AVGAGWFDP (SEQ ID |
| | NO: 4) | (SEQ ID NO: 5) | NO: 12) |
| X28 | NYDVN (SEQ ID | GIIPIFGTANYAQKFQG | AVGAGWFDP (SEQ ID |
| | NO: 4) | (SEQ ID NO: 5) | NO: 12) |
| X29 | DYYIH (SEQ ID | WMNPNSGNTGYAQKFQ | EGSSGWYVDY (SEQ ID |
| (PA-43) | NO: 38) | G (SEQ ID NO: 58) | NO: 39) |
| X30 | NYDVN (SEQ ID | GIIPIFGTANYAQKFQG | GVGAGWFDP (SEQ ID |
| | NO: 4) | (SEQ ID NO: 5) | NO: 6) |
| X31 | NYDVN (SEQ ID | GIIPIFGTANYAQKFQG | GVGAGWFDP (SEQ ID |
| | NO: 4) | (SEQ ID NO: 5) | NO: 6) |
| X32 | HYDVN (SEQ ID | GIIPIFGTANYAQKFQG | AVGAGWFDP (SEQ ID |
| | NO: 10) | (SEQ ID NO: 5) | NO: 12) |
| X33 | HYDVN (SEQ ID | GIIPIFGTANYAQKFQG | AVGAGWFDP (SEQ ID |
| | NO: 10) | (SEQ ID NO: 5) | NO: 12) |
| X34 | NYDIN (SEQ ID | GIIPIFGAPVYAQDFQG | ADGAGWFDP |
| (A6) | NO: 200) | (SEQ ID NO: 201) | (SEQ ID NO: 202) |

¹ The VH CDRs in Table 1 are determined according to Kabat.

TABLE 2
VL CDR Amino Acid Sequences ²

| Anti- | VL CDR1 | VL CDR2 | VL CDR3 |
|---|---|---|---|
| body | (SEQ ID NO:) | (SEQ ID NO:) | (SEQ ID NO:) |
| X0 | KSSQSVLYSSNNKNYLA | WASTRES (SEQ | QQYYSTPYT |
| (PA-29) | (SEQ ID NO: 13) | ID NO: 14) | (SEQ ID NO: 15) |
| X1 | KSSQSVLYSSNNKNYLA | WASTRES (SEQ | QQYYSTPYT |
| | (SEQ ID NO: 13) | ID NO: 14) | (SEQ ID NO: 15) |
| X2 | KSSQSVLYSSNNKNYLA | WASTRES (SEQ | QQYYSTPYT |
| | (SEQ ID NO: 13) | ID NO: 14) | (SEQ ID NO: 15) |
| X3 | KSSQSVLYSSNNKNYLA | WASTRES (SEQ | QQYFEEPYT |
| | (SEQ ID NO: 13) | ID NO: 14) | (SEQ ID NO: 25) |
| X4 | KSSQSVYFSPNNKNYLA | WASTRES (SEQ | QQYFEEPYT |
| | (SEQ ID NO: 19) | ID NO: 14) | (SEQ ID NO: 25) |
| X5 | KSSQSVFFDVNNKNYLA | WASTRES (SEQ | QQYFEEPYT |
| | (SEQ ID NO: 20) | ID NO: 14) | (SEQ ID NO: 25) |
| X6 | KSSQSVYFDVNNKNYLA | WASTRES (SEQ | QQYFEEPYT |
| | (SEQ ID NO: 21) | ID NO: 14) | (SEQ ID NO: 25) |
| X7 | KSSQSVFFDENNKNYLA | WASTRES (SEQ | QQYFEEPYT |
| | (SEQ ID NO: 22) | ID NO: 14) | (SEQ ID NO: 25) |
| X8 | KSSQSVWFSPNNKNYLA | WASTRES (SEQ | QQYFEEPYT |
| | (SEQ ID NO: 27) | ID NO: 14) | (SEQ ID NO: 25) |
| X9 | KSSQSVFFDVNNKNYLA | WASTRES (SEQ | QQYYEEPYT |
| | (SEQ ID NO: 20) | ID NO: 14) | (SEQ ID NO: 26) |
| X10 | KSSQSVFFDENNKNYLA | WASTRES (SEQ | QQYYEEPYT |
| | (SEQ ID NO: 22) | ID NO: 14) | (SEQ ID NO: 26) |
| X11 | KSSQSVYFSENNKNYLA | WASTRES (SEQ | QQYYEEPYT |
| | (SEQ ID NO: 32) | ID NO: 14) | (SEQ ID NO: 26) |
| X12 | KSSQSVLYSSNNKNYLA | WASTRES (SEQ | QQYFEEPYT |
| | (SEQ ID NO: 13) | ID NO: 14) | (SEQ ID NO: 25) |
| X13 | KSSQSVLYSSNNKNYLA | WASTRES (SEQ | QQYFEEPYT |
| | (SEQ ID NO: 13) | ID NO: 14) | (SEQ ID NO: 25) |
| X14 | KSSQSVYFSPNNKNYLA | WASTRES (SEQ | QQYFEEPYT |
| | (SEQ ID NO: 19) | ID NO: 14) | (SEQ ID NO: 25) |
| X15 | KSSQSVFFDVNNKNYLA | WASTRES (SEQ | QQYFEEPYT |
| | (SEQ ID NO: 20) | ID NO: 14) | (SEQ ID NO: 25) |
| X16 | KSSQSVYFDVNNKNYLA | WASTRES (SEQ | QQYFEEPYT |
| | (SEQ ID NO: 21) | ID NO: 14) | (SEQ ID NO: 25) |
| X17 | KSSQSVFFDENNKNYLA | WASTRES (SEQ | QQYFEEPYT |
| | (SEQ ID NO: 22) | ID NO: 14) | (SEQ ID NO: 25) |
| X18 | KSSQSVWFDPNNKNYLA | WASTRES (SEQ | QQYFEEPYT |
| | (SEQ ID NO: 33) | ID NO: 14) | (SEQ ID NO: 25) |
| X19 | KSSQSVFFDENNKNYLA | WASTRES (SEQ | QQYYEEPYT |
| | (SEQ ID NO: 22) | ID NO: 14) | (SEQ ID NO: 26) |
| X20 | KSSQSVYFSENNKNYLA | WASTRES (SEQ | QQYYEEPYT |
| | (SEQ ID NO: 32) | ID NO: 14) | (SEQ ID NO: 26) |
| X21 | KSSQSVLYSSNNKNYLA | WASTRES (SEQ | QQYFEEPYT |
| | (SEQ ID NO: 13) | ID NO: 14) | (SEQ ID NO: 25) |
| X22 | KSSQSVLYSSNNKNYLA | WASTRES (SEQ | QQYYEEPYT |
| | (SEQ ID NO: 13) | ID NO: 14) | (SEQ ID NO: 26) |
| X23 | KSSQSVYFSPNNKNYLA | WASTRES (SEQ | QQYYSTPYT |
| | (SEQ ID NO: 19) | ID NO: 14) | (SEQ ID NO: 15) |
| X24 | KSSQSVYFDVNNKNYLA | WASTRES (SEQ | QQYYSTPYT (SED |
| | (SEQ ID NO: 21) | ID NO: 14) | (SEQ ID NO: 15) |
| X25 | KSSQSVLYSSNNKNYLA | WASTRES (SEQ | QQYFEEPYT |
| | (SEQ ID NO: 13) | ID NO: 14) | (SEQ ID NO: 25) |
| X26 | KSSQSVLYSSNNKNYLA | WASTRES (SEQ | QQYYEEPYT |
| | (SEQ ID NO: 13) | ID NO: 14) | (SEQ ID NO: 26) |
| X27 | KSSQSVYFSPNNKNYLA | WASTRES (SEQ | QQYYSTPYT |
| | (SEQ ID NO: 19) | ID NO: 14) | (SEQ ID NO: 15) |
| X28 | KSSQSVYFDVNNKNYLA | WASTRES (SEQ | QQYYSTPYT |
| | (SEQ ID NO: 21) | ID NO: 14) | (SEQ ID NO: 15) |
| X29 | QASQDISQFLN | DAYSLQS | QQANSFPLT |
| (PA-43) | (SEQ ID NO: 16) | (SEQ IDNO: 17) | (SEQ ID NO: 18) |
| X30 | KSSQSVYFDVNNKNYLA | WASTRES | CQQYYEEPYT |
| | (SEQ ID NO: 21) | (SEQ ID NO: 14) | (SEQ ID NO: 141) |
| X31 | KSSQSVLFDVNNKNYLA | WASTRES | CQQYYEEPYT |
| | (SEQ ID NO: 140) | (SEQ ID NO: 14) | (SEQ ID NO: 141) |
| X32 | KSSQSVFFDVNNKNYLA | WASTRES | CQQYYEEPYT |
| | (SEQ ID NO: 20) | (SEQ ID NO: 14) | (*SEQ ID NO: 141) |
| X33 | KSSQSVLFDVNNKNYLA | WASTRES | CQQYYEEPYT |
| | (SEQ ID NO: 140) | (SEQ ID NO: 14) | (SEQ ID NO: 141) |
| X34 | KSSQSVFYSSNNQNYLA | WASTRES | QQYYSSPLT |
| (A6) | (SEQ ID NO: 203) | (SEQ ID NO: 14) | (SEQ ID NO: 204) |

² The VL CDRs in Table 2 are determined according to Kabat.

In some aspects, an antibody or antigen-binding fragment thereof described herein binds to human PILRA and comprises the VH of an antibody listed in Table 3.

TABLE 3
Variable Heavy Chain (VH) Amino Acid Sequences

| Antibody | VH Amino Acid Sequence (SEQ ID NO:) |
|---|---|
| X0 (PA-29) | QVQLVQSGAEVKKPGSSVKVSCKASGYNFLNYDVNWVRQAPGQGL |
| | EWMGGIIPIFGTANYAQKFQGRVTITADESTSTAYMELSSLRSEDTAV |
| | YYCATGVGAGWFDPWGQGTLVTVSS (SEQ ID NO: 28) |
| X1 | QVQLVQSGAEVKKPGSSVKVSCKASGYNFLHYDVNWVRQAPGQGL |
| | EWMGGIIPIFGTANYAQKFQGRVTITADESTSTAYMELSSLRSEDTAV |
| | YYCATAVGAGWFDTWGQGTLVTVSS (SEQ ID NO: 30) |
| X2 | QVQLVQSGAEVKKPGSSVKVSCKASGYNFLHYDVNWVRQAPGQGL |
| | EWMGGIIPIFGTANYAQKFQGRVTITADESTSTAYMELSSLRSEDTAV |
| | YYCATAVGAGWFDPWGQGTLVTVSS (SEQ ID NO: 31) |
| X3 | QVQLVQSGAEVKKPGSSVKVSCKASGYNFLNYDVNWVRQAPGQGL |
| | EWMGGIIPIFGTANYAQKFQGRVTITADESTSTAYMELSSLRSEDTAV |
| | YYCATGVGAGWFDPWGQGTLVTVSS (SEQ ID NO: 28) |
| X4 | QVQLVQSGAEVKKPGSSVKVSCKASGYNFLNYDVNWVRQAPGQGL |
| | EWMGGIIPIFGTANYAQKFQGRVTITADESTSTAYMELSSLRSEDTAV |
| | YYCATGVGAGWFDPWGQGTLVTVSS (SEQ ID NO: 28) |
| X5 | QVQLVQSGAEVKKPGSSVKVSCKASGYNFLNYDVNWVRQAPGQGL |
| | EWMGGIIPIFGTANYAQKFQGRVTITADESTSTAYMELSSLRSEDTAV |
| | YYCATGVGAGWFDPWGQGTLVTVSS (SEQ ID NO: 28) |
| X6 | QVQLVQSGAEVKKPGSSVKVSCKASGYNFLNYDVNWVRQAPGQGL |
| | EWMGGIIPIFGTANYAQKFQGRVTITADESTSTAYMELSSLRSEDTAV |
| | YYCATGVGAGWFDPWGQGTLVTVSS (SEQ ID NO: 28) |
| X7 | QVQLVQSGAEVKKPGSSVKVSCKASGYNFLNYDVNWVRQAPGQGL |
| | EWMGGIIPIFGTANYAQKFQGRVTITADESTSTAYMELSSLRSEDTAV |
| | YYCATGVGAGWFDPWGQGTLVTVSS (SEQ ID NO: 28) |
| X8 | QVQLVQSGAEVKKPGSSVKVSCKASGYNFLNYDVNWVRQAPGQGL |
| | EWMGGIIPIFGTANYAQKFQGRVTITADESTSTAYMELSSLRSEDTAV |
| | YYCATGVGAGWFDPWGQGTLVTVSS (SEQ ID NO: 28) |
| X9 | QVQLVQSGAEVKKPGSSVKVSCKASGYNFLNYDVNWVRQAPGQGL |
| | EWMGGIIPIFGTANYAQKFQGRVTITADESTSTAYMELSSLRSEDTAV |
| | YYCATGVGAGWFDPWGQGTLVTVSS (SEQ ID NO: 28) |
| X10 | QVQLVQSGAEVKKPGSSVKVSCKASGYNFLNYDVNWVRQAPGQGL |
| | EWMGGIIPIFGTANYAQKFQGRVTITADESTSTAYMELSSLRSEDTAV |
| | YYCATGVGAGWFDPWGQGTLVTVSS (SEQ ID NO: 28) |
| X11 | QVQLVQSGAEVKKPGSSVKVSCKASGYNFLNYDVNWVRQAPGQGL |
| | EWMGGIIPIFGTANYAQKFQGRVTITADESTSTAYMELSSLRSEDTAV |
| | YYCATGVGAGWFDPWGQGTLVTVSS (SEQ ID NO: 28) |
| X12 | QVQLVQSGAEVKKPGSSVKVSCKASGYNFLHYDVNWVRQAPGQGL |
| | EWMGGIIPIFGTANYAQKFQGRVTITADESTSTAYMELSSLRSEDTAV |
| | YYCATAVGAGWFDTWGQGTLVTVSS (SEQ ID NO: 30) |
| X13 | QVQLVQSGAEVKKPGSSVKVSCKASGYNFLHYDVNWVRQAPGQGL |
| | EWMGGIIPIFGTANYAQKFQGRVTITADESTSTAYMELSSLRSEDTAV |
| | YYCATAVGAGWFDPWGQGTLVTVSS (SEQ ID NO: 31) |
| X14 | QVQLVQSGAEVKKPGSSVKVSCKASGYNFLHYDVNWVRQAPGQGL |
| | EWMGGIIPIFGTANYAQKFQGRVTITADESTSTAYMELSSLRSEDTAV |
| | YYCATAVGAGWFDTWGQGTLVTVSS (SEQ ID NO: 30) |
| X15 | QVQLVQSGAEVKKPGSSVKVSCKASGYNFLHYDVNWVRQAPGQGL |
| | EWMGGIIPIFGTANYAQKFQGRVTITADESTSTAYMELSSLRSEDTAV |
| | YYCATAVGAGWFDTWGQGTLVTVSS (SEQ ID NO: 30) |
| X16 | QVQLVQSGAEVKKPGSSVKVSCKASGYNFLHYDVNWVRQAPGQGL |
| | EWMGGIIPIFGTANYAQKFQGRVTITADESTSTAYMELSSLRSEDTAV |
| | YYCATAVGAGWFDTWGQGTLVTVSS (SEQ ID NO: 30) |
| X17 | QVQLVQSGAEVKKPGSSVKVSCKASGYNFLHYDVNWVRQAPGQGL |
| | EWMGGIIPIFGTANYAQKFQGRVTITADESTSTAYMELSSLRSEDTAV |
| | YYCATAVGAGWFDTWGQGTLVTVSS (SEQ ID NO: 30) |
| X18 | QVQLVQSGAEVKKPGSSVKVSCKASGYNFLHYDVNWVRQAPGQGL |
| | EWMGGIIPIFGTANYAQKFQGRVTITADESTSTAYMELSSLRSEDTAV |
| | YYCATAVGAGWFDTWGQGTLVTVSS (SEQ ID NO: 30) |
| X19 | QVQLVQSGAEVKKPGSSVKVSCKASGYNFLHYDVNWVRQAPGQGL |
| | EWMGGIIPIFGTANYAQKFQGRVTITADESTSTAYMELSSLRSEDTAV |
| | YYCATAVGAGWFDPWGQGTLVTVSS (SEQ ID NO: 31) |
| X20 | QVQLVQSGAEVKKPGSSVKVSCKASGYNFLHYDVNWVRQAPGQGL |
| | EWMGGIIPIFGTANYAQKFQGRVTITADESTSTAYMELSSLRSEDTAV |
| | YYCATAVGAGWFDPWGQGTLVTVSS (SEQ ID NO: 31) |
| X21 | QVQLVQSGAEVKKPGSSVKVSCKASGYNFLHYDVNWVRQAPGQGL |
| | EWMGGIIPIFGTANYAQKFQGRVTITADESTSTAYMELSSLRSEDTAV |
| | YYCATGVGAGWFDPWGQGTLVTVSS (SEQ ID NO: 50) |
| X22 | QVQLVQSGAEVKKPGSSVKVSCKASGYNFLHYDVNWVRQAPGQGL |
| | EWMGGIIPIFGTANYAQKFQGRVTITADESTSTAYMELSSLRSEDTAV |
| | YYCATGVGAGWFDPWGQGTLVTVSS (SEQ ID NO: 50) |
| X23 | QVQLVQSGAEVKKPGSSVKVSCKASGYNFLHYDVNWVRQAPGQGL |
| | EWMGGIIPIFGTANYAQKFQGRVTITADESTSTAYMELSSLRSEDTAV |
| | YYCATGVGAGWFDPWGQGTLVTVSS (SEQ ID NO: 50) |
| X24 | QVQLVQSGAEVKKPGSSVKVSCKASGYNFLHYDVNWVRQAPGQGL |
| | EWMGGIIPIFGTANYAQKFQGRVTITADESTSTAYMELSSLRSEDTAV |
| | YYCATGVGAGWFDPWGQGTLVTVSS (SEQ ID NO: 50) |
| X25 | QVQLVQSGAEVKKPGSSVKVSCKASGYNFLNYDVNWVRQAPGQGL |
| | EWMGGIIPIFGTANYAQKFQGRVTITADESTSTAYMELSSLRSEDTAV |
| | YYCATAVGAGWFDPWGQGTLVTVSS (SEQ ID NO: 54) |
| X26 | QVQLVQSGAEVKKPGSSVKVSCKASGYNFLNYDVNWVRQAPGQGL |
| | EWMGGIIPIFGTANYAQKFQGRVTITADESTSTAYMELSSLRSEDTAV |
| | YYCATAVGAGWFDPWGQGTLVTVSS (SEQ ID NO: 54) |
| X27 | QVQLVQSGAEVKKPGSSVKVSCKASGYNFLNYDVNWVRQAPGQGL |
| | EWMGGIIPIFGTANYAQKFQGRVTITADESTSTAYMELSSLRSEDTAV |
| | YYCATAVGAGWFDPWGQGTLVTVSS (SEQ ID NO: 54) |
| X28 | QVQLVQSGAEVKKPGSSVKVSCKASGYNFLNYDVNWVRQAPGQGL |
| | EWMGGIIPIFGTANYAQKFQGRVTITADESTSTAYMELSSLRSEDTAV |
| | YYCATAVGAGWFDPWGQGTLVTVSS (SEQ ID NO: 54) |
| X29 (PA-43) | QVQLVQSGAEVKKPGASVKVSCKASGYTFTDYYIHWVRQAPGQGL |
| | EWMGWMNPNSGNTGYAQKFQGRVTMTRDTSTSTVYMELSSLRSED |
| | TAVYYCAREGSSGWYVDYWGQGTLVTVSS (SEQ IDNO: 29) |
| X30 | QVQLVQSGAEVKKPGSSVKVSCKASGYNFLNYDVNWVRQAPGQGL |
| | EWMGGIIPIFGTANYAQKFQGRVTITADESTSTAYMELSSLRSEDTAV |
| | YYCATGVGAGWFDPWGQGTLVTVSS (SEQ ID NO: 28) |
| X31 | QVQLVQSGAEVKKPGSSVKVSCKASGYNFLNYDVNWVRQAPGQGL |
| | EWMGGIIPIFGTANYAQKFQGRVTITADESTSTAYMELSSLRSEDTAV |
| | YYCATGVGAGWFDPWGQGTLVTVSS (SEQ ID NO: 28) |
| X32 | QVQLVQSGAEVKKPGSSVKVSCKASGYNFLHYDVNWVRQAPGQGL |
| | EWMGGIIPIFGTANYAQKFQGRVTITADESTSTAYMELSSLRSEDTAV |
| | YYCATAVGAGWFDPWGQGTLVTVSS (SEQ ID NO: 31) |
| X33 | QVQLVQSGAEVKKPGSSVKVSCKASGYNFLHYDVNWVRQAPGQGL |
| | EWMGGIIPIFGTANYAQKFQGRVTITADESTSTAYMELSSLRSEDTAV |
| | YYCATAVGAGWFDPWGQGTLVTVSS (SEQ ID NO: 31) |
| X34 (A6) | QVQLVQSGAEVKKPGSSVKVSCKASGYTFINYDINWVRQAPGQGLE |
| | WMGGIIPIFGAPVYAQDFQGRVTITADESTSTAYMELSSLRSEDTAV |
| | YYCAIADGAGWFDPWGQGTLVTVSS (SEQ ID NO: 205) |

In some aspects, an antibody or antigen-binding fragment thereof described herein binds to human PILRA and comprises the VL of an antibody listed in Table 4.

TABLE 4
Variable Light Chain (VL) Amino Acid Sequences

| Antibody | VL Amino Acid Sequence (SEQ ID NO:) |
|---|---|
| X0 (PA-29) | DIVMTQSPDSLAVSLGERATINCKSSQSVLYSSNNKNYLAWYQQKPG |
| | QPPKLLIYWASTRESGVPDRFSGSGSGTDFTLTISSLQAEDVAVYYCQ |
| | QYYSTPYTFGQGTRLEIK (SEQ ID NO: 60) |
| X1 | DIVMTQSPDSLAVSLGERATINCKSSQSVLYSSNNKNYLAWYQQKPG |
| | QPPKLLIYWASTRESGVPDRFSGSGSGTDFTLTISSLQAEDVAVYYCQ |
| | QYYSTPYTFGQGTRLEIK (SEQ ID NO: 60) |
| X2 | DIVMTQSPDSLAVSLGERATINCKSSQSVLYSSNNKNYLAWYQQKPG |
| | QPPKLLIYWASTRESGVPDRFSGSGSGTDFTLTISSLQAEDVAVYYCQ |
| | QYYSTPYTFGQGTRLEIK (SEQ ID NO: 60) |
| X3 | DIVMTQSPDSLAVSLGERATINCKSSQSVLYSSNNKNYLAWYQQKPG |
| | QPPKLLIYWASTRESGVPDRFSGSGSGTDFTLTISSLQAEDVAVYYCQ |
| | QYFEEPYTFGQGTRLEIK (SEQ ID NO: 63) |
| X4 | DIVMTQSPDSLAVSLGERATINCKSSQSVYFSPNNKNYLAWYQQKPG |
| | QPPKLLIYWASTRESGVPDRFSGSGSGTDFTLTISSLQAEDVAVYYCQ |
| | QYFEEPYTFGQGTRLEIK (SEQ ID NO: 64) |
| X5 | DIVMTQSPDSLAVSLGERATINCKSSQSVFFDVNNKNYLAWYQQKPG |
| | QPPKLLIYWASTRESGVPDRFSGSGSGTDFTLTISSLQAEDVAVYYCQ |
| | QYFEEPYTFGQGTRLEIK (SEQ ID NO: 65) |
| X6 | DIVMTQSPDSLAVSLGERATINCKSSQSVYFDVNNKNYLAWYQQKP |
| | GQPPKLLIYWASTRESGVPDRFSGSGSGTDFTLTISSLQAEDVAVYYC |
| | QQYFEEPYTFGQGTRLEIK (SEQ ID NO: 66) |
| X7 | DIVMTQSPDSLAVSLGERATINCKSSQSVFFDENNKNYLAWYQQKPG |
| | QPPKLLIYWASTRESGVPDRFSGSGSGTDFTLTISSLQAEDVAVYYCQ |
| | QYFEEPYTFGQGTRLEIK (SEQ ID NO: 67) |
| X8 | DIVMTQSPDSLAVSLGERATINCKSSQSVWFSPNNKNYLAWYQQKPG |
| | QPPKLLIYWASTRESGVPDRFSGSGSGTDFTLTISSLQAEDVAVYYCQ |
| | QYFEEPYTFGQGTRLEIK (SEQ ID NO: 68) |
| X9 | DIVMTQSPDSLAVSLGERATINCKSSQSVFFDVNNKNYLAWYQQKPG |
| | QPPKLLIYWASTRESGVPDRFSGSGSGTDFTLTISSLQAEDVAVYYCQ |
| | QYYEEPYTFGQGTRLEIK (SEQ ID NO: 69) |
| X10 | DIVMTQSPDSLAVSLGERATINCKSSQSVFFDENNKNYLAWYQQKPG |
| | QPPKLLIYWASTRESGVPDRFSGSGSGTDFTLTISSLQAEDVAVYYCQ |
| | QYYEEPYTFGQGTRLEIK (SEQ ID NO: 70) |
| X11 | DIVMTQSPDSLAVSLGERATINCKSSQSVYFSENNKNYLAWYQQKPG |
| | QPPKLLIYWASTRESGVPDRFSGSGSGTDFTLTISSLQAEDVAVYYCQ |
| | QYYEEPYTFGQGTRLEIK (SEQ ID NO: 71) |
| X12 | DIVMTQSPDSLAVSLGERATINCKSSQSVLYSSNNKNYLAWYQQKPG |
| | QPPKLLIYWASTRESGVPDRFSGSGSGTDFTLTISSLQAEDVAVYYCQ |
| | QYFEEPYTFGQGTRLEIK (SEQ ID NO: 63) |
| X13 | DIVMTQSPDSLAVSLGERATINCKSSQSVLYSSNNKNYLAWYQQKPG |
| | QPPKLLIYWASTRESGVPDRFSGSGSGTDFTLTISSLQAEDVAVYYCQ |
| | QYFEEPYTFGQGTRLEIK (SEQ ID NO: 63) |
| X14 | DIVMTQSPDSLAVSLGERATINCKSSQSVYFSPNNKNYLAWYQQKPG |
| | QPPKLLIYWASTRESGVPDRFSGSGSGTDFTLTISSLQAEDVAVYYCQ |
| | QYFEEPYTFGQGTRLEIK (SEQ ID NO: 64) |
| X15 | DIVMTQSPDSLAVSLGERATINCKSSQSVFFDVNNKNYLAWYQQKPG |
| | QPPKLLIYWASTRESGVPDRFSGSGSGTDFTLTISSLQAEDVAVYYCQ |
| | QYFEEPYTFGQGTRLEIK (SEQ ID NO: 65) |
| X16 | DIVMTQSPDSLAVSLGERATINCKSSQSVYFDVNNKNYLAWYQQKP |
| | GQPPKLLIYWASTRESGVPDRFSGSGSGTDFTLTISSLQAEDVAVYYC |
| | QQYFEEPYTFGQGTRLEIK (SEQ ID NO: 66) |
| X17 | DIVMTQSPDSLAVSLGERATINCKSSQSVFFDENNKNYLAWYQQKPG |
| | QPPKLLIYWASTRESGVPDRFSGSGSGTDFTLTISSLQAEDVAVYYCQ |
| | QYFEEPYTFGQGTRLEIK (SEQ ID NO: 67) |
| X18 | DIVMTQSPDSLAVSLGERATINCKSSQSVWFDPNNKNYLAWYQQKP |
| | GQPPKLLIYWASTRESGVPDRFSGSGSGTDFTLTISSLQAEDVAVYYC |
| | QQYFEEPYTFGQGTRLEIK (SEQ ID NO: 78) |
| X19 | DIVMTQSPDSLAVSLGERATINCKSSQSVFFDENNKNYLAWYQQKPG |
| | QPPKLLIYWASTRESGVPDRFSGSGSGTDFTLTISSLQAEDVAVYYCQ |
| | QYYEEPYTFGQGTRLEIK (SEQ ID NO: 70) |
| X20 | DIVMTQSPDSLAVSLGERATINCKSSQSVYFSENNKNYLAWYQQKPG |
| | QPPKLLIYWASTRESGVPDRFSGSGSGTDFTLTISSLQAEDVAVYYCQ |
| | QYYEEPYTFGQGTRLEIK (SEQ ID NO: 71) |
| X21 | DIVMTQSPDSLAVSLGERATINCKSSQSVLYSSNNKNYLAWYQQKPG |
| | QPPKLLIYWASTRESGVPDRFSGSGSGTDFTLTISSLQAEDVAVYYCQ |
| | QYFEEPYTFGQGTRLEIK (SEQ ID NO: 63) |
| X22 | DIVMTQSPDSLAVSLGERATINCKSSQSVLYSSNNKNYLAWYQQKPG |
| | QPPKLLIYWASTRESGVPDRFSGSGSGTDFTLTISSLQAEDVAVYYCQ |
| | QYYEEPYTFGQGTRLEIK (SEQ ID NO: 82) |
| X23 | DIVMTQSPDSLAVSLGERATINCKSSQSVYFSPNNKNYLAWYQQKPG |
| | QPPKLLIYWASTRESGVPDRFSGSGSGTDFTLTISSLQAEDVAVYYCQ |
| | QYYSTPYTFGQGTRLEIK (SEQ ID NO: 83) |
| X24 | DIVMTQSPDSLAVSLGERATINCKSSQSVYFDVNNKNYLAWYQQKP |
| | GQPPKLLIYWASTRESGVPDRFSGSGSGTDFTLTISSLQAEDVAVYYC |
| | QQYYSTPYTFGQGTRLEIK (SEQ ID NO: 84) |
| X25 | DIVMTQSPDSLAVSLGERATINCKSSQSVLYSSNNKNYLAWYQQKPG |
| | QPPKLLIYWASTRESGVPDRFSGSGSGTDFTLTISSLQAEDVAVYYCQ |
| | QYFEEPYTFGQGTRLEIK (SEQ ID NO: 63) |
| X26 | DIVMTQSPDSLAVSLGERATINCKSSQSVLYSSNNKNYLAWYQQKPG |
| | QPPKLLIYWASTRESGVPDRFSGSGSGTDFTLTISSLQAEDVAVYYCQ |
| | QYYEEPYTFGQGTRLEIK (SEQ ID NO: 82) |
| X27 | DIVMTQSPDSLAVSLGERATINCKSSQSVYFSPNNKNYLAWYQQKPG |
| | QPPKLLIYWASTRESGVPDRFSGSGSGTDFTLTISSLQAEDVAVYYCQ |
| | QYYSTPYTFGQGTRLEIK (SEQ ID NO: 83) |
| X28 | DIVMTQSPDSLAVSLGERATINCKSSQSVYFDVNNKNYLAWYQQKP |
| | GQPPKLLIYWASTRESGVPDRFSGSGSGTDFTLTISSLQAEDVAVYYC |
| | QQYYSTPYTFGQGTRLEIK (SEQ ID NO: 84) |
| X29 (PA-43) | DIQMTQSPSSLSASVGDRVTITCQASQDISQFLNWYQQKPGKAPKLLI |
| | YDAYSLQSGVPSRFSGSGSGTDFTLTISSLQPEDFATYYCQQANSFPLT |
| | FGQGTKVEIK (SEQ ID NO: 59) |
| X30 | DIVMTQSPDSLAVSLGERATINCKSSQSVYFDVNNKNYLAWYQQKP |
| | GQPPKLLIYWASTRESGVPDRFSGSGSGTDFTLTISSLQAEDVAVYYC |
| | QQYYEEPYTFGQGTRLEIK (SEQ ID NO: 142) |
| X31 | DIVMTQSPDSLAVSLGERATINCKSSQSVLFDVNNKNYLAWYQQKPG |
| | QPPKLLIYWASTRESGVPDRFSGSGSGTDFTLTISSLQAEDVAVYYCQ |
| | QYYEEPYTFGQGTRLEIK (SEQ ID NO: 143) |
| X32 | DIVMTQSPDSLAVSLGERATINCKSSQSVFFDVNNKNYLAWYQQKPG |
| | QPPKLLIYWASTRESGVPDRFSGSGSGTDFTLTISSLQAEDVAVYYCQ |
| | QYYEEPYTFGQGTRLEIK (SEQ ID NO: 69) |
| X33 | DIVMTQSPDSLAVSLGERATINCKSSQSVLFDVNNKNYLAWYQQKPG |
| | QPPKLLIYWASTRESGVPDRFSGSGSGTDFTLTISSLQAEDVAVYYCQ |
| | QYYEEPYTFGQGTRLEIK (SEQ ID NO: 143) |
| X34 (A6) | DIVMTQSPDSLAVSLGERATINCKSSQSVFYSSNNQNYLAWYQQKPG |
| | QPPKLLIYWASTRESGVPDRFSGSGSGTDFTLTISSLQAEDVAVYYCQ |
| | QYYSSPLTFGQGTRLEIK (SEQ ID NO: 206) |

In some aspects, an antibody or antigen-binding fragment thereof described herein binds to human PILRA and comprises the VH and the VL of an antibody listed in Tables 3 and 4 (i.e., the VH of the antibody listed in Table 3 and the VL of the same antibody listed in Table 4).

In some aspects, an antibody or antigen-binding fragment thereof described herein binds to human PILRA and comprises one, two, three or all of the VH framework regions of an antibody listed in Table 5.

TABLE 5
VH FR Amino Acid Sequences ³

| Anti- | VH FR1 | VH FR2 | VH FR3 | VH FR4 |
|---|---|---|---|---|
| body | (SEQ ID NO:) | (SEQ ID NO:) | (SEQ ID NO:) | (SEQ ID NO:) |
| X0 | QVQLVQSGAEVK | WVRQAPGQGL | RVTITADESTSTAY | WGQGTLVT |
| (PA-29) | KPGSSVKVSCKAS | EWMG (SEQ ID | MELSSLRSEDTAVY | VSS (SEQ ID |
| | GYNFL (SEQ ID | NO: 90) | YCAT (SEQ ID | NO: 92) |
| | NO: 89) | | NO: 91) | |
| X1 | QVQLVQSGAEVK | WVRQAPGQGL | RVTITADESTSTAY | WGQGTLVT |
| | KPGSSVKVSCKAS | EWMG (SEQ ID | MELSSLRSEDTAVY | VSS (SEQ ID |
| | GYNFL (SEQ ID | NO: 90) | YCAT (SEQ ID | NO: 92) |
| | NO: 89) | | NO: 91) | |
| X2 | QVQLVQSGAEVK | WVRQAPGQGL | RVTITADESTSTAY | WGQGTLVT |
| | KPGSSVKVSCKAS | EWMG (SEQ ID | MELSSLRSEDTAVY | VSS (SEQ ID |
| | GYNFL (SEQ ID | NO: 90) | YCAT (SEQ ID | NO: 92) |
| | NO: 89) | | NO: 91) | |
| X3 | QVQLVQSGAEVK | WVRQAPGQGL | RVTITADESTSTAY | WGQGTLVT |
| | KPGSSVKVSCKAS | EWMG (SEQ ID | MELSSLRSEDTAVY | VSS (SEQ ID |
| | GYNFL (SEQ ID | NO: 90) | YCAT (SEQ ID | NO: 92) |
| | NO: 89) | | NO: 91) | |
| X4 | QVQLVQSGAEVK | WVRQAPGQGL | RVTITADESTSTAY | WGQGTLVT |
| | KPGSSVKVSCKAS | EWMG (SEQ ID | MELSSLRSEDTAVY | VSS (SEQ ID |
| | GYNFL (SEQ ID | NO: 90) | YCAT (SEQ ID | NO: 92) |
| | NO: 89) | | NO: 91) | |
| X5 | QVQLVQSGAEVK | WVRQAPGQGL | RVTITADESTSTAY | WGQGTLVT |
| | KPGSSVKVSCKAS | EWMG (SEQ ID | MELSSLRSEDTAVY | VSS (SEQ ID |
| | GYNFL (SEQ ID | NO: 90) | YCAT (SEQ ID | NO: 92) |
| | NO: 89) | | NO: 91) | |
| X6 | QVQLVQSGAEVK | WVRQAPGQGL | RVTITADESTSTAY | WGQGTLVT |
| | KPGSSVKVSCKAS | EWMG (SEQ ID | MELSSLRSEDTAVY | VSS (SEQ ID |
| | GYNFL (SEQ ID | NO: 90) | YCAT (SEQ ID | NO: 92) |
| | NO: 89) | | NO: 91) | |
| X7 | QVQLVQSGAEVK | WVRQAPGQGL | RVTITADESTSTAY | WGQGTLVT |
| | KPGSSVKVSCKAS | EWMG (SEQ ID | MELSSLRSEDTAVY | VSS (SEQ ID |
| | GYNFL (SEQ ID | NO: 90) | YCAT (SEQ ID | NO: 92) |
| | NO: 89) | | NO: 91) | |
| X8 | QVQLVQSGAEVK | WVRQAPGQGL | RVTITADESTSTAY | WGQGTLVT |
| | KPGSSVKVSCKAS | EWMG (SEQ ID | MELSSLRSEDTAVY | VSS (SEQ ID |
| | GYNFL (SEQ ID | NO: 90) | YCAT (SEQ ID | NO: 92) |
| | NO: 89) | | NO: 91) | |
| X9 | QVQLVQSGAEVK | WVRQAPGQGL | RVTITADESTSTAY | WGQGTLVT |
| | KPGSSVKVSCKAS | EWMG (SEQ ID | MELSSLRSEDTAVY | VSS (SEQ ID |
| | GYNFL (SEQ ID | NO: 90) | YCAT (SEQ ID | NO: 92) |
| | NO: 89) | | NO: 91) | |
| X10 | QVQLVQSGAEVK | WVRQAPGQGL | RVTITADESTSTAY | WGQGTLVT |
| | KPGSSVKVSCKAS | EWMG (SEQ ID | MELSSLRSEDTAVY | VSS (SEQ ID |
| | GYNFL (SEQ ID | NO: 90) | YCAT (SEQ ID | NO: 92) |
| | NO: 89) | | NO: 91) | |
| X11 | QVQLVQSGAEVK | WVRQAPGQGL | RVTITADESTSTAY | WGQGTLVT |
| | KPGSSVKVSCKAS | EWMG (SEQ ID | MELSSLRSEDTAVY | VSS (SEQ ID |
| | GYNFL (SEQ ID | NO: 90) | YCAT (SEQ ID | NO: 92) |
| | NO: 89) | | NO: 91) | |
| X12 | QVQLVQSGAEVK | WVRQAPGQGL | RVTITADESTSTAY | WGQGTLVT |
| | KPGSSVKVSCKAS | EWMG (SEQ ID | MELSSLRSEDTAVY | VSS (SEQ ID |
| | GYNFL (SEQ ID | NO: 90) | YCAT (SEQ ID | NO: 92) |
| | NO: 89) | | NO: 91) | |
| X13 | QVQLVQSGAEVK | WVRQAPGQGL | RVTITADESTSTAY | WGQGTLVT |
| | KPGSSVKVSCKAS | EWMG (SEQ ID | MELSSLRSEDTAVY | VSS (SEQ ID |
| | GYNFL (SEQ ID | NO: 90) | YCAT (SEQ ID | NO: 92) |
| | NO: 89) | | NO: 91) | |
| X14 | QVQLVQSGAEVK | WVRQAPGQGL | RVTITADESTSTAY | WGQGTLVT |
| | KPGSSVKVSCKAS | EWMG (SEQ ID | MELSSLRSEDTAVY | VSS (SEQ ID |
| | GYNFL (SEQ ID | NO: 90) | YCAT (SEQ ID | NO: 92) |
| | NO: 89) | | NO: 91) | |
| X15 | QVQLVQSGAEVK | WVRQAPGQGL | RVTITADESTSTAY | WGQGTLVT |
| | KPGSSVKVSCKAS | EWMG (SEQ ID | MELSSLRSEDTAVY | VSS (SEQ ID |
| | GYNFL (SEQ ID | NO: 90) | YCAT (SEQ ID | NO: 92) |
| | NO: 89) | | NO: 91) | |
| X16 | QVQLVQSGAEVK | WVRQAPGQGL | RVTITADESTSTAY | WGQGTLVT |
| | KPGSSVKVSCKAS | EWMG (SEQ ID | MELSSLRSEDTAVY | VSS (SEQ ID |
| | GYNFL (SEQ ID | NO: 90) | YCAT (SEQ ID | NO: 92) |
| | NO: 89) | | NO: 91) | |
| X17 | QVQLVQSGAEVK | WVRQAPGQGL | RVTITADESTSTAY | WGQGTLVT |
| | KPGSSVKVSCKAS | EWMG (SEQ ID | MELSSLRSEDTAVY | VSS (SEQ ID |
| | GYNFL (SEQ ID | NO: 90) | YCAT (SEQ ID | NO: 92) |
| | NO: 89) | | NO: 91) | |
| X18 | QVQLVQSGAEVK | WVRQAPGQGL | RVTITADESTSTAY | WGQGTLVT |
| | KPGSSVKVSCKAS | EWMG (SEQ ID | MELSSLRSEDTAVY | VSS (SEQ ID |
| | GYNFL (SEQ ID | NO: 90) | YCAT (SEQ ID | NO: 92) |
| | NO: 89) | | NO: 91) | |
| X19 | QVQLVQSGAEVK | WVRQAPGQGL | RVTITADESTSTAY | WGQGTLVT |
| | KPGSSVKVSCKAS | EWMG (SEQ ID | MELSSLRSEDTAVY | VSS (SEQ ID |
| | GYNFL (SEQ ID | NO: 90) | YCAT (SEQ ID | NO: 92) |
| | NO: 89) | | NO: 91) | |
| X20 | QVQLVQSGAEVK | WVRQAPGQGL | RVTITADESTSTAY | WGQGTLVT |
| | KPGSSVKVSCKAS | EWMG (SEQ ID | MELSSLRSEDTAVY | VSS (SEQ ID |
| | GYNFL (SEQ ID | NO: 90) | YCAT (SEQ ID | NO: 92) |
| | NO: 89) | | NO: 91) | |
| X21 | QVQLVQSGAEVK | WVRQAPGQGL | RVTITADESTSTAY | WGQGTLVT |
| | KPGSSVKVSCKAS | EWMG (SEQ ID | MELSSLRSEDTAVY | VSS (SEQ ID |
| | GYNFL (SEQ ID | NO: 90) | YCAT (SEQ ID | NO: 92) |
| | NO: 89) | | NO: 91) | |
| X22 | QVQLVQSGAEVK | WVRQAPGQGL | RVTITADESTSTAY | WGQGTLVT |
| | KPGSSVKVSCKAS | EWMG (SEQ ID | MELSSLRSEDTAVY | VSS (SEQ ID |
| | GYNFL (SEQ ID | NO: 90) | YCAT (SEQ ID | NO: 92) |
| | NO: 89) | | NO: 91) | |
| X23 | QVQLVQSGAEVK | WVRQAPGQGL | RVTITADESTSTAY | WGQGTLVT |
| | KPGSSVKVSCKAS | EWMG (SEQ ID | MELSSLRSEDTAVY | VSS (SEQ ID |
| | GYNFL (SEQ ID | NO: 90) | YCAT (SEQ ID | NO: 92) |
| | NO: 89) | | NO: 91) | |
| X24 | QVQLVQSGAEVK | WVRQAPGQGL | RVTITADESTSTAY | WGQGTLVT |
| | KPGSSVKVSCKAS | EWMG (SEQ ID | MELSSLRSEDTAVY | VSS (SEQ ID |
| | GYNFL (SEQ ID | NO: 90) | YCAT (SEQ ID | NO: 92) |
| | NO: 89) | | NO: 91) | |
| X25 | QVQLVQSGAEVK | WVRQAPGQGL | RVTITADESTSTAY | WGQGTLVT |
| | KPGSSVKVSCKAS | EWMG (SEQ ID | MELSSLRSEDTAVY | VSS (SEQ ID |
| | GYNFL (SEQ ID | NO: 90) | YCAT (SEQ ID | NO: 92) |
| | NO: 89) | | NO: 91) | |
| X26 | QVQLVQSGAEVK | WVRQAPGQGL | RVTITADESTSTAY | WGQGTLVT |
| | KPGSSVKVSCKAS | EWMG (SEQ ID | MELSSLRSEDTAVY | VSS (SEQ ID |
| | GYNFL (SEQ ID | NO: 90) | YCAT (SEQ ID | NO: 92) |
| | NO: 89) | | NO: 91) | |
| X27 | QVQLVQSGAEVK | WVRQAPGQGL | RVTITADESTSTAY | WGQGTLVT |
| | KPGSSVKVSCKAS | EWMG (SEQ ID | MELSSLRSEDTAVY | VSS (SEQ ID |
| | GYNFL (SEQ ID | NO: 90) | YCAT (SEQ ID | NO: 92) |
| | NO: 89) | | NO: 91) | |
| X28 | QVQLVQSGAEVK | WVRQAPGQGL | RVTITADESTSTAY | WGQGTLVT |
| | KPGSSVKVSCKAS | EWMG (SEQ ID | MELSSLRSEDTAVY | VSS (SEQ ID |
| | GYNFL (SEQ ID | NO: 90) | YCAT (SEQ ID | NO: 92) |
| | NO: 89) | | NO: 91) | |
| X29 | QVQLVQSGAEVK | WVRQAPGQGL | RVTMTRDTSTSTVY | WGQGTLVT |
| (PA-43) | KPGASVKVSCKAS | EWMG (SEQ ID | MELSSLRSEDTAVY | VSS (SEQ ID |
| | GYTFT (SEQ ID | NO: 90) | YCAR (SEQ ID | NO: 92) |
| | NO: 93) | | NO: 94) | |
| X30 | QVQLVQSGAEVK | WVRQAPGQGL | RVTITADESTSTAY | WGQGTLVT |
| | KPGSSVKVSCKAS | EWMG (SEQ ID | MELSSLRSEDTAVY | VSS (SEQ ID |
| | GYNFL (SEQ ID | NO: 90) | YCAT (SEQ ID | NO: 92) |
| | NO: 89) | | NO: 91) | |
| X31 | QVQLVQSGAEVK | WVRQAPGQGL | RVTITADESTSTAY | WGQGTLVT |
| | KPGSSVKVSCKAS | EWMG (SEQ ID | MELSSLRSEDTAVY | VSS (SEQ ID |
| | GYNFL (SEQ ID | NO: 90) | YCAT (SEQ ID | NO: 92) |
| | NO: 89) | | NO: 91) | |
| X32 | QVQLVQSGAEVK | WVRQAPGQGL | RVTITADESTSTAY | WGQGTLVT |
| | KPGSSVKVSCKAS | EWMG (SEQ ID | MELSSLRSEDTAVY | VSS (SEQ ID |
| | GYNFL (SEQ ID | NO: 90) | YCAT (SEQ ID | NO: 92) |
| | NO: 89) | | NO: 91) | |
| X33 | QVQLVQSGAEVK | WVRQAPGQGL | RVTITADESTSTAY | WGQGTLVT |
| | KPGSSVKVSCKAS | EWMG (SEQ ID | MELSSLRSEDTAVY | VSS (SEQ ID |
| | GYNFL (SEQ ID | NO: 90) | YCAT (SEQ ID | NO: 92) |
| | NO: 89) | | NO: 91) | |
| X34 | QVQLVQSGAEVK | WVRQAPGQGL | RVTITADESTSTAY | WGQGTLVT |
| (A6) | KPGSSVKVSCKAS | EWMG (SEQ ID | MELSSLRSEDTAVY | VSS (SEQ ID |
| | GYTFI (SEQ ID | NO: 90) | YCAI (SEQ ID | NO: 92) |
| | NO: 207) | | NO: 208) | |

³ The VH framework regions described in Table 5 are determined based upon the boundaries of the Kabat numbering system for CDRs. In other words, the VH CDRs are determined by Kabat and the framework regions are the amino acid residues surrounding the CDRs in the variable region in the format FR1, CDR1, FR2, CDR2, FR3, CDR3, and FR4.

In some aspects, an antibody or antigen-binding fragment thereof described herein binds to human PILRA and comprises one, two, three or all of the VL framework regions of an antibody listed in Table 6.

TABLE 6
VL FR Amino Acid Sequences ⁴

| | VL FR1 | VL FR2 | VL FR3 | VL FR4 |
|---|---|---|---|---|
| Antibody | (SEQ ID NO:) | (SEQ ID NO:) | (SEQ ID NO:) | (SEQ ID NO:) |
| X0 | DIVMTQSPDSLAV | WYQQKPGQPP | GVPDRFSGSGSGT | FGQGTRLEI |
| (PA-29) | SLGERATINC (SEQ | KLLIY (SEQ ID | DFTLTISSLQAED | K (SEQ ID |
| | ID NO: 95) | NO: 96) | VAVYYC (SEQ ID | NO: 98) |
| | | | NO: 97) | |
| X1 | DIVMTQSPDSLAV | WYQQKPGQPP | GVPDRFSGSGSGT | FGQGTRLEI |
| | SLGERATINC (SEQ | KLLIY (SEQ ID | DFTLTISSLQAED | K (SEQ ID |
| | ID NO: 95) | NO: 96) | VAVYYC (SEQ ID | NO: 98) |
| | | | NO: 97) | |
| X2 | DIVMTQSPDSLAV | WYQQKPGQPP | GVPDRFSGSGSGT | FGQGTRLEI |
| | SLGERATINC (SEQ | KLLIY (SEQ ID | DFTLTISSLQAED | K (SEQ ID |
| | ID NO: 95) | NO: 96) | VAVYYC (SEQ ID | NO: 98) |
| | | | NO: 97) | |
| X3 | DIVMTQSPDSLAV | WYQQKPGQPP | GVPDRFSGSGSGT | FGQGTRLEI |
| | SLGERATINC (SEQ | KLLIY (SEQ ID | DFTLTISSLQAED | K (SEQ ID |
| | ID NO: 95) | NO: 96) | VAVYYC (SEQ ID | NO: 98) |
| | | | NO: 97) | |
| X4 | DIVMTQSPDSLAV | WYQQKPGQPP | GVPDRFSGSGSGT | FGQGTRLEI |
| | SLGERATINC (SEQ | KLLIY (SEQ ID | DFTLTISSLQAED | K (SEQ ID |
| | ID NO: 95) | NO: 96) | VAVYYC (SEQ ID | NO: 98) |
| | | | NO: 97) | |
| X5 | DIVMTQSPDSLAV | WYQQKPGQPP | GVPDRFSGSGSGT | FGQGTRLEI |
| | SLGERATINC (SEQ | KLLIY (SEQ ID | DFTLTISSLQAED | K (SEQ ID |
| | ID NO: 95) | NO: 96) | VAVYYC (SEQ ID | NO: 98) |
| | | | NO: 97) | |
| X6 | DIVMTQSPDSLAV | WYQQKPGQPP | GVPDRFSGSGSGT | FGQGTRLEI |
| | SLGERATINC (SEQ | KLLIY (SEQ ID | DFTLTISSLQAED | K (SEQ ID |
| | ID NO: 95) | NO: 96) | VAVYYC (SEQ ID | NO: 98) |
| | | | NO: 97) | |
| X7 | DIVMTQSPDSLAV | WYQQKPGQPP | GVPDRFSGSGSGT | FGQGTRLEI |
| | SLGERATINC (SEQ | KLLIY (SEQ ID | DFTLTISSLQAED | K (SEQ ID |
| | ID NO: 95) | NO: 96) | VAVYYC (SEQ ID | NO: 98) |
| | | | NO: 97) | |
| X8 | DIVMTQSPDSLAV | WYQQKPGQPP | GVPDRFSGSGSGT | FGQGTRLEI |
| | SLGERATINC (SEQ | KLLIY (SEQ ID | DFTLTISSLQAED | K (SEQ ID |
| | ID NO: 95) | NO: 96) | VAVYYC (SEQ ID | NO: 98) |
| | | | NO: 97) | |
| X9 | DIVMTQSPDSLAV | WYQQKPGQPP | GVPDRFSGSGSGT | FGQGTRLEI |
| | SLGERATINC (SEQ | KLLIY (SEQ ID | DFTLTISSLQAED | K (SEQ ID |
| | ID NO: 95) | NO: 96) | VAVYYC (SEQ ID | NO: 98) |
| | | | NO: 97) | |
| X10 | DIVMTQSPDSLAV | WYQQKPGQPP | GVPDRFSGSGSGT | FGQGTRLEI |
| | SLGERATINC (SEQ | KLLIY (SEQ ID | DFTLTISSLQAED | K (SEQ ID |
| | ID NO: 95) | NO: 96) | VAVYYC (SEQ ID | NO: 98) |
| | | | NO: 97) | |
| X11 | DIVMTQSPDSLAV | WYQQKPGQPP | GVPDRFSGSGSGT | FGQGTRLEI |
| | SLGERATINC (SEQ | KLLIY (SEQ ID | DFTLTISSLQAED | K (SEQ ID |
| | ID NO: 95) | NO: 96) | VAVYYC (SEQ ID | NO: 98) |
| | | | NO: 97) | |
| X12 | DIVMTQSPDSLAV | WYQQKPGQPP | GVPDRFSGSGSGT | FGQGTRLEI |
| | SLGERATINC (SEQ | KLLIY (SEQ ID | DFTLTISSLQAED | K (SEQ ID |
| | ID NO: 95) | NO: 96) | VAVYYC (SEQ ID | NO: 98) |
| | | | NO: 97) | |
| X13 | DIVMTQSPDSLAV | WYQQKPGQPP | GVPDRFSGSGSGT | FGQGTRLEI |
| | SLGERATINC (SEQ | KLLIY (SEQ ID | DFTLTISSLQAED | K (SEQ ID |
| | ID NO: 95) | NO: 96) | VAVYYC (SEQ ID | NO: 98) |
| | | | NO: 97) | |
| X14 | DIVMTQSPDSLAV | WYQQKPGQPP | GVPDRFSGSGSGT | FGQGTRLEI |
| | SLGERATINC (SEQ | KLLIY (SEQ ID | DFTLTISSLQAED | K (SEQ ID |
| | ID NO: 95) | NO: 96) | VAVYYC (SEQ ID | NO: 98) |
| | | | NO: 97) | |
| X15 | DIVMTQSPDSLAV | WYQQKPGQPP | GVPDRFSGSGSGT | FGQGTRLEI |
| | SLGERATINC (SEQ | KLLIY (SEQ ID | DFTLTISSLQAED | K (SEQ ID |
| | ID NO: 95) | NO: 96) | VAVYYC (SEQ ID | NO: 98) |
| | | | NO: 97) | |
| X16 | DIVMTQSPDSLAV | WYQQKPGQPP | GVPDRFSGSGSGT | FGQGTRLEI |
| | SLGERATINC (SEQ | KLLIY (SEQ ID | DFTLTISSLQAED | K (SEQ ID |
| | ID NO: 95) | NO: 96) | VAVYYC (SEQ ID | NO: 98) |
| | | | NO: 97) | |
| X17 | DIVMTQSPDSLAV | WYQQKPGQPP | GVPDRFSGSGSGT | FGQGTRLEI |
| | SLGERATINC (SEQ | KLLIY (SEQ ID | DFTLTISSLQAED | K (SEQ ID |
| | ID NO: 95) | NO: 96) | VAVYYC (SEQ ID | NO: 98) |
| | | | NO: 97) | |
| X18 | DIVMTQSPDSLAV | WYQQKPGQPP | GVPDRFSGSGSGT | FGQGTRLEI |
| | SLGERATINC (SEQ | KLLIY (SEQ ID | DFTLTISSLQAED | K (SEQ ID |
| | ID NO: 95) | NO: 96) | VAVYYC (SEQ ID | NO: 98) |
| | | | NO: 97) | |
| X19 | DIVMTQSPDSLAV | WYQQKPGQPP | GVPDRFSGSGSGT | FGQGTRLEI |
| | SLGERATINC (SEQ | KLLIY (SEQ ID | DFTLTISSLQAED | K (SEQ ID |
| | ID NO: 95) | NO: 96) | VAVYYC (SEQ ID | NO: 98) |
| | | | NO: 97) | |
| X20 | DIVMTQSPDSLAV | WYQQKPGQPP | GVPDRFSGSGSGT | FGQGTRLEI |
| | SLGERATINC (SEQ | KLLIY (SEQ ID | DFTLTISSLQAED | K (SEQ ID |
| | ID NO: 95) | NO: 96) | VAVYYC (SEQ ID | NO: 98) |
| | | | NO: 97) | |
| X21 | DIVMTQSPDSLAV | WYQQKPGQPP | GVPDRFSGSGSGT | FGQGTRLEI |
| | SLGERATINC (SEQ | KLLIY (SEQ ID | DFTLTISSLQAED | K (SEQ ID |
| | ID NO: 95) | NO: 96) | VAVYYC (SEQ ID | NO: 98) |
| | | | NO: 97) | |
| X22 | DIVMTQSPDSLAV | WYQQKPGQPP | GVPDRFSGSGSGT | FGQGTRLEI |
| | SLGERATINC (SEQ | KLLIY (SEQ ID | DFTLTISSLQAED | K (SEQ ID |
| | ID NO: 95) | NO: 96) | VAVYYC (SEQ ID | NO: 98) |
| | | | NO: 97) | |
| X23 | DIVMTQSPDSLAV | WYQQKPGQPP | GVPDRFSGSGSGT | FGQGTRLEI |
| | SLGERATINC (SEQ | KLLIY (SEQ ID | DFTLTISSLQAED | K (SEQ ID |
| | ID NO: 95) | NO: 96) | VAVYYC (SEQ ID | NO: 98) |
| | | | NO: 97) | |
| X24 | DIVMTQSPDSLAV | WYQQKPGQPP | GVPDRFSGSGSGT | FGQGTRLEI |
| | SLGERATINC (SEQ | KLLIY (SEQ ID | DFTLTISSLQAED | K (SEQ ID |
| | ID NO: 95) | NO: 96) | VAVYYC (SEQ ID | NO: 98) |
| | | | NO: 97) | |
| X25 | DIVMTQSPDSLAV | WYQQKPGQPP | GVPDRFSGSGSGT | FGQGTRLEI |
| | SLGERATINC (SEQ | KLLIY (SEQ ID | DFTLTISSLQAED | K (SEQ ID |
| | ID NO: 95) | NO: 96) | VAVYYC (SEQ ID | NO: 98) |
| | | | NO: 97) | |
| X26 | DIVMTQSPDSLAV | WYQQKPGQPP | GVPDRFSGSGSGT | FGQGTRLEI |
| | SLGERATINC (SEQ | KLLIY (SEQ ID | DFTLTISSLQAED | K (SEQ ID |
| | ID NO: 95) | NO: 96) | VAVYYC (SEQ ID | NO: 98) |
| | | | NO: 97) | |
| X27 | DIVMTQSPDSLAV | WYQQKPGQPP | GVPDRFSGSGSGT | FGQGTRLEI |
| | SLGERATINC (SEQ | KLLIY (SEQ ID | DFTLTISSLQAED | K (SEQ ID |
| | ID NO: 95) | NO: 96) | VAVYYC (SEQ ID | NO: 98) |
| | | | NO: 97) | |
| X28 | DIVMTQSPDSLAV | WYQQKPGQPP | GVPDRFSGSGSGT | FGQGTRLEI |
| | SLGERATINC (SEQ | KLLIY (SEQ ID | DFTLTISSLQAED | K (SEQ ID |
| | ID NO: 95) | NO: 96) | VAVYYC (SEQ ID | NO: 98) |
| | | | NO: 97) | |
| X29 (PA- | DIQMTQSPSSLSAS | WYQQKPGKAP | GVPSRFSGSGSGT | FGQGTK VEI |
| 43) | VGDRVTITC (SEQ | KLLIY (SEQ ID | DFTLTISSLQPEDF | K (SEQ ID |
| | ID NO: 99) | NO: 100) | ATYYC (SEQ ID | NO: 102) |
| | | | NO: 101) | |
| X30 | DIVMTQSPDSLAV | WYQQKPGQPP | GVPDRFSGSGSGT | FGQGTRLEI |
| | SLGERATINC (SEQ | KLLIY (SEQ ID | DFTLTISSLQAED | K (SEQ ID |
| | ID NO: 95) | NO: 96) | VAVYY (SEQ ID | NO: 98) |
| | | | NO: 144) | |
| X31 | DIVMTQSPDSLAV | WYQQKPGQPP | GVPDRFSGSGSGT | FGQGTRLEI |
| | SLGERATINC (SEQ | KLLIY (SEQ ID | DFTLTISSLQAED | K (SEQ ID |
| | ID NO: 95) | NO: 96) | VAVYY (SEQ ID | NO: 98) |
| | | | NO: 144) | |
| X32 | DIVMTQSPDSLAV | WYQQKPGQPP | GVPDRFSGSGSGT | FGQGTRLEI |
| | SLGERATINC (SEQ | KLLIY (SEQ ID | DFTLTISSLQAED | K (SEQ ID |
| | ID NO: 95) | NO: 96) | VAVYY (SEQ ID | NO: 98) |
| | | | NO: 144) | |
| X33 | DIVMTQSPDSLAV | WYQQKPGQPP | GVPDRFSGSGSGT | FGQGTRLEI |
| | SLGERATINC (SEQ | KLLIY (SEQ ID | DFTLTISSLQAED | K (SEQ ID |
| | ID NO: 95) | NO: 96) | VAVYY (SEQ ID | NO: 98) |
| | | | NO: 144) | |
| X34 (A6) | DIVMTQSPDSLAV | WYQQKPGQPP | GVPDRFSGSGSGT | FGQGTRLEI |
| | SLGERATINC (SEQ | KLLIY (SEQ ID | DFTLTISSLQAED | K (SEQ ID |
| | ID NO: 95) | NO: 96) | VAVYYC (SEQ ID | NO: 98) |
| | | | NO: 97) | |

⁴ The VL framework regions described in Table 6 are determined based upon the boundaries of the Kabat numbering system for CDRs. In other words, the VL CDRs are determined by Kabat and the framework regions are the amino acid residues surrounding the CDRs in the variable region in the format FR1, CDR1, FR2, CDR2, FR3, CDR3, and FR4.

In some aspects, an antibody or antigen-binding fragment thereof described herein binds to human PILRA and comprises the four VH framework regions and the four VL framework regions of an antibody listed in Tables 5 and 6 (i.e., the four VH framework regions of the antibody listed in Table 5 and the four VL framework regions of the same antibody listed in Table 6.)

In certain aspects, an antibody or antigen-binding fragment thereof described herein may be described by its VL domain alone, or its VH domain alone, or by its 3 VL CDRs alone, or its 3 VH CDRs alone. See, for example, Rader C et al., (1998) PNAS 95:8910-8915, which is incorporated herein by reference in its entirety, describing the humanization of the mouse anti-αvβ3 antibody by identifying a complementing light chain or heavy chain, respectively, from a human light chain or heavy chain library, resulting in humanized antibody variants having affinities as high or higher than the affinity of the original antibody. See also Clackson T et al., (1991) Nature 352:624-628, which is incorporated herein by reference in its entirety, describing methods of producing antibodies that bind a specific antigen by using a specific VL domain (or VH domain) and screening a library for the complementary variable domains. The screen produced 14 new partners for a specific VH domain and 13 new partners for a specific VL domain, which were strong binders, as determined by ELISA. See also Kim S J & Hong H J, (2007) J Microbiol 45:572-577, which is incorporated herein by reference in its entirety, describing methods of producing antibodies that bind a specific antigen by using a specific VH domain and screening a library (e.g., human VL library) for complementary VL domains; the selected VL domains in turn could be used to guide selection of additional complementary (e.g., human) VH domains.

In certain aspects, the CDRs of an antibody or antigen-binding fragment thereof can be determined according to the Chothia numbering scheme, which refers to the location of immunoglobulin structural loops (see, e.g., Chothia C & Lesk A M, (1987), J Mol Biol 196:901-917; A1-Lazikani B et al., (1997) J Mol Biol 273:927-948; Chothia C et al., (1992) J Mol Biol 227:799-817; Tramontano A et al., (1990) J Mol Biol 215 (1): 175-82; and U.S. Pat. No. 7,709,226). Typically, when using the Kabat numbering convention, the Chothia CDR-H1 loop is present at heavy chain amino acids 26 to 32, 33, or 34, the Chothia CDR-H2 loop is present at heavy chain amino acids 52 to 56, and the Chothia CDR-H3 loop is present at heavy chain amino acids 95 to 102, while the Chothia CDR-L1 loop is present at light chain amino acids 24 to 34, the Chothia CDR-L2 loop is present at light chain amino acids 50 to 56, and the Chothia CDR-L3 loop is present at light chain amino acids 89 to 97. The end of the Chothia CDR-H1 loop when numbered using the Kabat numbering convention varies between H32 and H34 depending on the length of the loop (this is because the Kabat numbering scheme places the insertions at H35A and H35B; if neither 35A nor 35B is present, the loop ends at 32; if only 35A is present, the loop ends at 33; if both 35A and 35B are present, the loop ends at 34).

In certain aspects, provided herein are antibodies and antigen-binding fragments thereof that specifically bind to human PILRA and comprise the Chothia VH and VL CDRs of an antibody listed in Tables 3 and 4. In some aspects, antibodies or antigen-binding fragments thereof that specifically bind to human PILRA comprise one or more CDRs, in which the Chothia and Kabat CDRs have the same amino acid sequence. In some aspects, provided herein are antibodies and antigen-binding fragments thereof that specifically bind to human PILRA and comprise combinations of Kabat CDRs and Chothia CDRs.

In certain aspects, the CDRs of an antibody or antigen-binding fragment thereof can be determined according to the IMGT numbering system as described in Lefranc M-P, (1999) The Immunologist 7:132-136 and Lefranc M-P et al., (1999) Nucleic Acids Res 27:209-212. According to the IMGT numbering scheme, VH-CDR1 is at positions 26 to 35, VH-CDR2 is at positions 51 to 57, VH-CDR3 is at positions 93 to 102, VL-CDR1 is at positions 27 to 32, VL-CDR2 is at positions 50 to 52, and VL-CDR3 is at positions 89 to 97. In some aspects, provided herein are antibodies and antigen-binding fragments thereof that specifically bind to human PILRA and comprise the IMGT VH and VL CDRs of an antibody listed in Tables 3 and 4, for example, as described in Lefranc M-P (1999) supra and Lefranc M-P et al., (1999) supra).

In certain aspects, the CDRs of an antibody or antigen-binding fragment thereof can be determined according to MacCallum R M et al., (1996) J Mol Biol 262:732-745. See also, e.g., Martin A. “Protein Sequence and Structure Analysis of Antibody Variable Domains,” in Antibody Engineering, Kontermann and Dübel, eds., Chapter 31, pp. 422-439, Springer-Verlag, Berlin (2001). In some aspects, provided herein are antibodies or antigen-binding fragments thereof that specifically bind to human PILRA and comprise VH and VL CDRs of an antibody listed in Tables 3 and 4 as determined by the method in MacCallum R M et al.

In certain aspects, the CDRs of an antibody or antigen-binding fragment thereof can be determined according to the AbM numbering scheme, which refers to AbM hypervariable regions, which represent a compromise between the Kabat CDRs and Chothia structural loops, and are used by Oxford Molecular's AbM antibody modeling software (Oxford Molecular Group, Inc.). In some aspects, provided herein are antibodies or antigen-binding fragments thereof that specifically bind to human PILRA and comprise VH and VL CDRs of an antibody listed in Tables 3 and 4 as determined by the AbM numbering scheme.

In some aspects, provided herein are antibodies that comprise a heavy chain and a light chain. With respect to the heavy chain, in some aspects, the heavy chain of an antibody described herein can be an alpha (α), delta (δ), epsilon (ε), gamma (γ) or mu (μ) heavy chain. In some aspects, the heavy chain of an antibody described can comprise a human alpha (α), delta (δ), epsilon (ε), gamma (γ) or mu (μ) heavy chain. In some aspects, an antibody described herein, which immunospecifically binds to human PILRA, comprises a heavy chain wherein the amino acid sequence of the VH domain comprises an amino acid sequence set forth in Table 3 and wherein the constant region of the heavy chain comprises the amino acid sequence of a human gamma (γ) heavy chain constant region. In some aspects, an antibody described herein, which immunospecifically binds to human PILRA, comprises a heavy chain wherein the amino acid sequence of the VH domain comprises an amino acid sequence set forth in Table 3 and wherein the constant region of the heavy chain comprises the amino acid sequence of an IgG1 heavy chain constant region. In some aspects, an antibody described herein, which immunospecifically binds to human PILRA, comprises a heavy chain wherein the amino acid sequence of the VH domain comprises an amino acid sequence set forth in Table 3 and wherein the constant region of the heavy chain comprises the amino acid sequence of an IgG2 (e.g., IgG2a or IgG2b) heavy chain constant region. In some aspects, an antibody described herein, which immunospecifically binds to human PILRA, comprises a heavy chain wherein the amino acid sequence of the VH domain comprises an amino acid sequence set forth in Table 3 and wherein the constant region of the heavy chain comprises the amino acid sequence of an IgG4 heavy chain constant region. In some aspects, an antibody described herein, which immunospecifically binds to human PILRA, comprises a heavy chain wherein the amino acid sequence of the VH domain comprises a sequence set forth in Table 3, and wherein the constant region of the heavy chain comprises the amino acid of a human heavy chain described herein or known in the art. Non-limiting examples of human constant region sequences have been described in the art, e.g., see U.S. Pat. No. 5,693,780 and Kabat E A et al., (1991) supra.

With respect to the light chain, in some aspects, the light chain of an antibody described herein is a kappa light chain. In some aspects, the light chain of an antibody described herein is a lambda light chain. In some aspects, the light chain of an antibody described herein is a human kappa light chain or a human lambda light chain.

In some aspects, an antibody described herein, which immunospecifically binds to a human PILRA, comprises a light chain wherein the amino acid sequence of the VL domain comprises a sequence set forth in Table 4, and wherein the constant region of the light chain comprises the amino acid sequence of a human kappa light chain constant region. In some aspects, an antibody described herein, which immunospecifically binds to human PILRA, comprises a light chain wherein the amino acid sequence of the VL domain comprises a sequence set forth in Table 4, and wherein the constant region of the light chain comprises the amino acid sequence of a human lambda light chain constant region. In some aspects, an antibody described herein, which immunospecifically binds to human PILRA, comprises a light chain wherein the amino acid sequence of the VL domain comprises a sequence set forth in Table 4, and wherein the constant region of the light chain comprises the amino acid sequence of a human kappa or lambda light chain constant region. Non-limiting examples of human constant region sequences have been described in the art, e.g., see U.S. Pat. No. 5,693,780 and Kabat E A et al., (1991) supra.

In some aspects, an antibody described herein, which immunospecifically binds to human PILRA comprises a VH domain and a VL domain comprising the amino acid sequence of any of the anti-human PILRA antibodies described herein, and wherein the constant regions comprise the amino acid sequences of the constant regions of an IgG, IgE, IgM, IgD, IgA, or IgY immunoglobulin molecule, or a human IgG, IgE, IgM, IgD, IgA, or IgY immunoglobulin molecule. In some aspects, an antibody described herein, which immunospecifically binds to human PILRA comprises a VH domain and a VL domain comprising the amino acid sequences of any of the anti-human PILRA antibodies described herein, and wherein the constant regions comprise the amino acid sequences of the constant regions of an IgG, IgE, IgM, IgD, IgA, or IgY immunoglobulin molecule, any class (e.g., IgG1, IgG2, IgG3, IgG4, IgA1, and IgA2), or any subclass (e.g., IgG2a and IgG2b) of immunoglobulin molecule. In some aspects, the constant regions comprise the amino acid sequences of the constant regions of a human IgG, IgE, IgM, IgD, IgA, or IgY immunoglobulin molecule, any class (e.g., IgG1, IgG2, IgG3, IgG4, IgA1, and IgA2), or any subclass (e.g., IgG2a and IgG2b) of immunoglobulin molecule.

Non-limiting examples of human constant regions are described in the art, e.g., see Kabat E A et al., (1991) supra.

### Exemplary Fc Domains

In some aspects, an anti-PILRA antibody, and in particular, an anti-human PILRA antibody as provided herein, may comprise an Fc domain. In some aspects, the Fc domain is a human IgG1, IgG2, IgG3, and/or IgG4 isotype.

In certain aspects, the Fc domain has an IgG1 isotype. In some aspects, an anti-PILRA antibody contains a murine IgG1 Fc domain. In some aspects, an anti-human PILRA antibody contains a human IgG1 Fc domain (hIgG1), e.g., as provided in SEQ ID NO:106.

(SEQ ID NO: 106)
EPKSCDKTHTCPPCPAPELLGGPSVFLFPPKPKDTLMISRTPEVT
CVVVDVSHEDPEVKFNWYVDGVEVHNAKTKPREEQYNSTYRVVSV
LTVLHQDWLNGKEYKCKVSNKALPAPIEKTISKAKGQPREPQVYT
LPPSRDELTKNQVSLTCLVKGFYPSDIAVEWESNGQPENNYKTTP
PVLDSDGSFFLYSKLTVDKSRWQQGNVFSCSVMHEALHNHYTQKS
LSLSPGK

In some aspects, the human IgG1 Fc domain of an anti-human PILRA antibody binds an activating Fc receptor. In certain aspects, the activating Fc receptor is selected from any one or more of FcγRI, FcγRIIa and IIc, and FcγRIIIa and IIIb.

In some aspects, the human IgG1 Fc domain of an anti-human PILRA antibody does not bind or has reduced binding to FcγRIII (CD16) and/or C1q. In some aspects, the human IgG1 Fc domain of an anti-human PILRA antibody has reduced antibody-dependent cellular cytotoxicity (ADCC) and/or complement binding activity, respectively, which in each case may reduce undesired killing of cells, e.g., myeloid cells, to which the anti-PILRA antibody binds. The above effects may be achieved by certain amino acid modifications, e.g., the “NSLF” mutations, in which a human IgG1 Fc domain contains the mutations N325S and L328F (by EU numbering of the IgG1 Fc domain), as shown, e.g., in SEQ ID NO:107. In another aspect, the human IgG1 Fc domain comprises a mutation corresponding to K322A (EU numbering), e.g., as provided in SEQ ID NO: 108.

(SEQ ID NO: 107)
EPKSCDKTHTCPPCPAPELLGGPSVFLFPPKPKDTLMISRTPEVT
CVVVDVSHEDPEVKFNWYVDGVEVHNAKTKPREEQYNSTYRVVSV
LTVLHQDWLNGKEYKCKVSSKAFPAPIEKTISKAKGQPREPQVYT
LPPSRDELTKNQVSLTCLVKGFYPSDIAVEWESNGQPENNYKTTP
PVLDSDGSFFLYSKLTVDKSRWQQGNVFSCSVMHEALHNHYTQKS
LSLSPGK
(SEQ ID NO: 108)
EPKSCDKTHTCPPCPAPELLGGPSVFLFPPKPKDTLMISRTPEVT
CVVVDVSHEDPEVKFNWYVDGVEVHNAKTKPREEQYNSTYRVVSV
LTVLHQDWLNGKEYKCAVSNKALPAPIEKTISKAKGQPREPQVYT
LPPSRDELTKNQVSLTCLVKGFYPSDIAVEWESNGQPENNYKTTP
PVLDSDGSFFLYSKLTVDKSRWQQGNVFSCSVMHEALHNHYTQKS
LSLSPGK

Exemplary modifications to the human IgG1 Fc domain are listed below in Table 7.

TABLE 7
Exemplary modifications to the human IgG1 Fc domain
Mutation (EU numbering scheme)

N325S and L328F (“NSLF”)
S267E and L328F (“SELF”)
P331S (“PS”)
P331S and E430G (“PSEG”)
K322A
L234A, L235A, and P331S (“LALAPS”)
(Substantially abolishes Fc binding to FcR)

In certain aspects of an anti-PILRA antibody provided herein, the Fc domain has an IgG2 isotype. In some aspects, an anti-PILRA antibody contains a murine IgG2 Fc domain, e.g., murine IgG2a (mIgG2a). In some aspects, an anti-human PILRA antibody contains a human IgG2 Fc domain (hIgG2). In some aspects, the human IgG2 Fc domain of an anti-human PILRA antibody binds an activating Fc receptor. In certain aspects, the activating Fc receptor is selected from any one or more of FcγRI, FcγRIIa and IIc, and FcγRIIIa and IIIb.

In certain aspects of an anti-PILRA antibody provided herein, the Fc domain has an IgG4 isotype. In some aspects, an anti-human PILRA antibody contains a human IgG4 Fc domain (hIgG4), e.g., as provided in SEQ ID NO: 109. In some aspects, the human IgG4 Fc region of the anti-human PILRA antibody binds an activating Fc receptor. In certain aspects, the activating Fc receptor is selected from any one or more of FcγRI, FcγRIIa and IIc, and FcγRIIIa and IIIb. In certain aspects, the human IgG4 Fc region comprises a mutation corresponding to S228P (by EU numbering), e.g., as provided in SEQ ID NO:110.

(SEQ ID NO: 109)
ESKYGPPCPSCPAPEFLGGPSVFLFPPKPKDTLMISRTPEVTCVV
VDVSQEDPEVQFNWYVDGVEVHNAKTKPREEQFNSTYRVVSVLTV
LHQDWLNGKEYKCKVSNKGLPSSIEKTISKAKGQPREPQVYTLPP
SQEEMTKNQVSLTCLVKGFYPSDIAVEWESNGQPENNYKTTPPVL
DSDGSFFLYSRLTVDKSRWQEGNVFSCSVMHEALHNHYTQKSLSL
SLGK
(SEQ ID NO: 110)
ESKYGPPCPPCPAPEFLGGPSVFLFPPKPKDTLMISRTPEVTCVV
VDVSQEDPEVQFNWYVDGVEVHNAKTKPREEQFNSTYRVVSVLTV
LHQDWLNGKEYKCKVSNKGLPSSIEKTISKAKGQPREPQVYTLPP
SQEEMTKNQVSLTCLVKGFYPSDIAVEWESNGQPENNYKTTPPVL
DSDGSFFLYSRLTVDKSRWQEGNVFSCSVMHEALHNHYTQKSLSL
SLGK

In some aspects, any of the constant region mutations or modifications described herein can be introduced into one or both heavy chain constant regions of an antibody or antigen-binding fragment thereof described herein having two heavy chain constant regions.

In some aspects, an antibody or antigen-binding fragment thereof described herein, which immunospecifically binds to human PILRA, comprises a heavy chain and a light chain, wherein (i) the heavy chain comprises a VH domain comprising the VH CDR1, VH CDR2, and VH CDR3 amino acid sequences of an antibody listed in Table 1; (ii) the light chain comprises a VL domain comprising the VL CDR1, VL CDR2, and VL CDR3 amino acid sequences of the same antibody listed in Table 2; (iii) the heavy chain further comprises a constant heavy chain domain comprising the amino acid sequence of the constant domain of a human IgG1 heavy chain; and (iv) the light chain further comprises a constant light chain domain comprising the amino acid sequence of the constant domain of a human kappa light chain.

In some aspects, an antibody or antigen-binding fragment thereof described herein, which immunospecifically binds to human PILRA, comprises a heavy chain and a light chain, wherein (i) the heavy chain comprises a VH domain comprising the amino acid sequence of an antibody listed in Table 3; (ii) the light chain comprises a VL domain comprising the amino acid sequence of the same antibody listed in Table 4; (iii) the heavy chain further comprises a constant heavy chain domain comprising the amino acid sequence of the constant domain of a human IgG1 heavy chain; and (iv) the light chain further comprises a constant light chain domain comprising the amino acid sequence of the constant domain of a human kappa light chain.

In some aspects, an antibody or antigen-binding fragment thereof described herein, which immunospecifically binds to human PILRA, comprises framework regions (e.g., framework regions of the VH domain and/or VL domain) that are human framework regions or derived from human framework regions. Non-limiting examples of human framework regions are described in the art, e.g., see Kabat E A et al., (1991) supra). In some aspects, an antibody or antigen-binding fragment thereof described herein comprises framework regions (e.g., framework regions of the VH domain and/or VL domain) that are primate (e.g., non-human primate) framework regions or derived from primate (e.g., non-human primate) framework regions.

In some aspects, an antibody or antigen-binding fragment thereof described herein, which immunospecifically binds to human PILRA, comprises one, two, three or four VH framework regions (FRs) having the amino acid sequences described herein for an antibody set forth in Table 5, supra. In some aspects, an antibody or antigen-binding fragment thereof described herein, which immunospecifically binds to human PILRA, comprises one, two, three or four VL framework regions (FRs) having the amino acid sequences described herein for an antibody set forth in Table 6, supra. In some aspects, an antibody or antigen-binding fragment thereof described herein, which immunospecifically binds to human PILRA, comprises one, two, three or four VH framework regions having the amino acid sequences described herein for an antibody set forth in Table 5, supra, and one, two, three or four VL framework regions having the amino acid sequences described herein for the same antibody set forth in Table 6, supra.

### Antibody Activities

In some aspects, an antibody or antigen-binding fragment thereof described herein, which immunospecifically binds to human PILRA, downregulates cell-surface PILRA. An anti-human PILRA antibody that downregulates PILRA would have the effect of decreasing inhibitory signaling that would otherwise result from engagement of cell surface PILRA with its ligand.

In some aspects, an antibody or antigen-binding fragment thereof described herein, which immunospecifically binds to human PILRA, downregulates cell-surface PILRA (e.g., human PILRA on primary human macrophages), as compared to the level of cell-surface PILRA in the absence of the antibody or fragment or in the presence of a control antibody or fragment. In some aspects, cell-surface PILRA is downregulated by at least 10%, at least 20%, at least 30%, at least 40%, or at least 50% in the presence of the anti-human PILRA antibody after 24 hours at 37° C., as compared to the level of cell surface PILRA in the absence of the antibody or fragment or in the presence of a control antibody or fragment after 24 hours at 37° C. In some aspects, cell-surface PILRA is downregulated by about 10% to about 50% in the presence of the anti-human PILRA antibody after 24 hours at 37° C., as compared to cell surface PILRA in the absence of the antibody or fragment or in the presence of a control antibody or fragment after 24 hours at 37° C. In some aspects, cell surface PILRA is downregulated by at least 30%, at least 40%, at least 50%, or at least 60% in the presence of the anti-human PILRA antibody after 24 hours at 37° C., e.g., as compared to the level of cell surface PILRA in the absence of the antibody or fragment or in the presence of a control antibody or fragment after 24 hours at 37° C. In some aspects, cell-surface PILRA is downregulated by about 30% to about 60% in the presence of the anti-human PILRA antibody after 24 hours at 37° C., e.g., as compared to the level of cell surface PILRA in the absence of the antibody or fragment or in the presence of a control antibody or fragment after 24 hours at 37° C. The percent downregulation can be calculated, for example, by normalizing the levels of cell surface PILRA detected after incubation at the indicated time points on ice versus 37° C., in the presence or absence of anti-human PILRA antibody, or in the presence of anti-human PILRA antibody or a control antibody.

Downregulation can be measured, for example, using the assay disclosed in Example 9. Downregulation can be measured, for example, by incubating human or cynomolgus primary monocytes with anti-human PILRA antibody or with no antibody or control antibody (e.g., for 24 hours at 37° C.) and detecting cell surface human PILRA. Cell surface human or cynomolgus PILRA can be detected, for example, using FACS. The downregulation can be dependent on the dose of anti-human PILRA antibody. In some embodiments, an antibody or antigen-binding fragment thereof described herein, which immunospecifically binds to human PILRA downregulates levels of cell surface PILRA at least 20% to at least 35% more than the X0 (PA-29) antibody.

In some aspects, an antibody or antigen-binding fragment thereof described herein, which immunospecifically binds to human PILRA, blocks binding of PILRA-Fc to human T-cells. An anti-human PILRA antibody that binds to PILRA-Fc and blocks binding of PILRA-Fc to T-cells is presumed to be blocking binding of PILRA-Fc to a PILRA ligand on T-cells. An antibody with this activity would likewise be expected to function in blocking the binding of endogenous PILRA to a PILRA ligand, thereby suppressing the inhibitory signaling of endogenous PILRA.

In some aspects, an antibody or antigen-binding fragment thereof described herein, which immunospecifically binds to human PILRA, blocks binding of PILRA-Fc to human T-cells by at least 70%, e.g., as compared to the binding of PILRA-Fc to human T-cells in the absence of the antibody or fragment or in the presence of a control antibody or fragment. In some aspects, an antibody or antigen-binding fragment thereof described herein, which immunospecifically binds to human PILRA, blocks binding of PILRA-Fc to human T-cells by at least 75%, e.g., as compared to the binding of PILRA-Fc to human T-cells in the presence of a control antibody. In some aspects, an antibody or antigen-binding fragment thereof described herein, which immunospecifically binds to human PILRA, blocks binding of PILRA-Fc to human T-cells by at least 80%, e.g., as compared to the binding of PILRA-Fc to human T-cells in the absence of the antibody or fragment or in the presence of a control antibody or fragment. In some aspects, an antibody or antigen-binding fragment thereof described herein, which immunospecifically binds to human PILRA, blocks binding of PILRA-Fc to human T-cells by at least 85%, e.g., as compared to the binding of PILRA-Fc to human T-cells in the absence of the antibody or fragment or in the presence of a control antibody or fragment. In some aspects, an antibody or antigen-binding fragment thereof described herein, which immunospecifically binds to human PILRA, blocks binding of PILRA-Fc to human T-cells by at least 90%, e.g., as compared to the binding of PILRA-Fc to human T-cells in the absence of the antibody or fragment or in the presence of a control antibody or fragment. In some aspects, an antibody or antigen-binding fragment thereof described herein, which immunospecifically binds to human PILRA, blocks binding of PILRA-Fc to human T-cells by at least 95%, e.g., as compared to the binding of PILRA-Fc to human T-cells in the absence of the antibody or fragment or in the presence of a control antibody or fragment. In some aspects, an antibody or antigen-binding fragment thereof described herein, which immunospecifically binds to human PILRA, blocks binding of PILRA-Fc to human T-cells by 100%, e.g., as compared to the binding of PILRA-Fc to human T-cells in the absence of the antibody or fragment or in the presence of a control antibody or fragment. In some aspects, an antibody or antigen-binding fragment thereof described herein, which immunospecifically binds to human PILRA, blocks binding of PILRA-Fc to human T-cells by about 70% to about 95%, or by about 70% to about 98%, e.g., as compared to the binding of PILRA-Fc to the human T-cells in the presence of a control antibody or fragment. In some aspects, an antibody or antigen-binding fragment thereof described herein, which immunospecifically binds to human PILRA, blocks binding of PILRA-Fc to human T-cells by about 80% to about 95% e.g., as compared to the binding of PILRA-Fc to human T-cells in the presence of a control antibody or fragment.

Blocking of binding can be measured, for example, by testing an antibody's inhibition of binding of PILRA-Fc to T cells. Blocking of binding can be measured, for example, by incubating an anti-human PILRA antibody or antigen-binding fragment thereof with about 5 μg/ml PILRA-Fc for 30 min at 4° C., then adding human T cells for 30 min at 4° C., washing the cells and detecting bound PILRA-Fc. The bound PILRA-Fc can be detected, for example, using flow cytometry. The blocking of binding can be dependent on the dose of anti-human PILRA antibody.

In some aspects, an antibody or antigen-binding fragment thereof described herein, which immunospecifically binds to human PILRA, blocks binding of PILRA to one or more PILRA ligands. PILRA ligands include glycosylated proteins, such as those with PTPXP, PTPXXP, PXTPXP, or PXTPXXP motif. Exemplary ligands include COLEC12, NPDC1, CLEC4G, and PIANP, as well as the HSV-1 glycoprotein B. Amino acid Arg126 in PILRA (SEQ ID NO: 1), is well known to be essential for sialic acid interaction (see e.g., Rathore et al., PLOS Genetics 14: e1007427 (2018)).

In some aspects, an anti-human PILRA antibody that blocks binding of PILRA to one or more of its ligands can be identified by testing the ability of the antibody to block binding of PILRA-Fc to one or more PILRA ligands, wherein such ligand is expressed in a soluble form or expressed on a cell surface. In some aspects, an anti-human PILRA antibody that blocks binding of PILRA to one or more of its ligands can be identified by testing the ability of the antibody to block binding of cells expressing cell surface PILRA to one or more PILRA ligands, wherein such ligand is expressed in a soluble form or expressed on a cell surface.

In some aspects, an antibody or antigen-binding fragment thereof described herein, which immunospecifically binds to human PILRA, blocks binding of PILRA-Fc to one or more PILRA ligands (including but not limited to those provided above), e.g., as compared to the binding in the absence of the antibody or fragment or in the presence of a control antibody or fragment. Blocking of binding can be measured, for example, using the assay disclosed in Example 7 (e.g., by testing an antibody's ability to modulate binding of ligand to cells expressing PILRA). Blocking of binding can be measured, for example, by incubating cells expressing PILRA (e.g., for about 30 min at about 4° C.), with about 5 μg/ml of the ligand, incubating again (e.g., for about 30 min at about 4° C.), washing the cells and detecting bound ligand. The bound ligand can be detected, for example, using flow cytometry. The blocking of binding can be dependent on the dose of anti-human PILRA antibody.

In some aspects, an antibody or antigen-binding fragment thereof described herein, which immunospecifically binds to human PILRA, activates myeloid cells (e.g., macrophages (such as “M1” macrophages), microglia, dendritic cells, neutrophils, granulocytes and other myeloid-derived cells such as myeloid-derived suppressor cells (“MDSCs”)). By activating myeloid cells, an anti-human PILRA antibody is capable of activating the innate immune system, e.g., which can promote an anti-tumor response and, in the case of microglia, can promote an environment in the CNS that counteracts neurodegenerative conditions.

In some aspects, an antibody or antigen-binding fragment thereof described herein, which immunospecifically binds to human PILRA, activates myeloid cells (such as those described above), as compared to the activation of myeloid cells in the absence of the antibody or fragment or in the presence of a control antibody or fragment. Activation of myeloid cells (e.g., MDSCs) can be assessed, for example, by detecting the amount of MIP1b produced by the cells in the presence of an anti-human PILRA antibody or antigen-binding fragment thereof compared to activation in the absence of the antibody or fragment or in the presence of a control antibody or fragment. The activation of the myeloid cells (e.g., MDSCs) can be dependent on the dose of anti-human PILRA antibody.

In some aspects, an antibody or antigen-binding fragment thereof described herein, which immunospecifically binds to human PILRA, promotes differentiation of myeloid cells (e.g., promotes differentiation of monocytes into macrophages (such as “M1” macrophages) or dendritic cells, or promotes differentiation of other myeloid precursors into microglia, dendritic cells, neutrophils and other myeloid-derived cells such as myeloid-derived suppressor cells (“MDSCs”)). By promoting the differentiation of myeloid cells, an anti-human PILRA antibody is capable of activating the innate immune system, e.g., which can promote an anti-tumor response (e.g., through M1 macrophages in the tumor microenvironment) and, in the case of microglia, can promote an environment in the CNS that counteracts neurodegenerative conditions.

In some aspects, an antibody or antigen-binding fragment thereof described herein, which immunospecifically binds to human PILRA, promotes differentiation of myeloid cells (such as those described above), compared to the differentiation of the myeloid cells in the absence of the antibody or fragment or in the presence of a control antibody or fragment. Differentiation of myeloid cells (e.g., MDSCs) can be assessed, for example, by detecting the amount of CD14^{lo}CD86^{hi} cells in the presence of an anti-human PILRA antibody or antigen-binding fragment thereof compared to differentiation in the absence of the antibody or fragment or in the presence of a control antibody or fragment. The differentiation of the myeloid cells (e.g., MDSCs) can be dependent on the dose of anti-human PILRA antibody.

### Antibodies That Bind Same Epitope/Competitively Inhibit

In another aspect, provided herein are antibodies or antigen-binding fragments thereof that bind to the same epitope of human PILRA as an antibody or antigen-binding fragment thereof described herein (e.g., X0-X34).

In another aspect, provided herein are antibodies or antigen-binding fragments thereof that bind to an overlapping epitope of human PILRA as an antibody or antigen-binding fragment thereof described herein (e.g., X0-X34). Antibodies that have overlapping epitopes contact at least one or more of the same amino acid residues of PILRA.

Competitive binding assays can be used to determine whether two antibodies bind to overlapping epitopes. Competitive binding can be determined in an assay in which an immunoglobulin to be tested inhibits specific binding of a reference antibody to a common antigen, such as PILRA (e.g., human PILRA). Numerous types of competitive binding assays are known, for example: solid phase direct or indirect radioimmunoassay (RIA), solid phase direct or indirect enzyme immunoassay (EIA), sandwich competition assay (see Stahli C et al., (1983) Methods Enzymol 9:242-253); solid phase direct biotin-avidin EIA (see Kirkland T N et al., (1986) J Immunol 137:3614-9); solid phase direct labeled assay, solid phase direct labeled sandwich assay (see Harlow E & Lane D, (1988) Antibodies: A Laboratory Manual, Cold Spring Harbor Press); solid phase direct label RIA using I-125 label (see Morel G A et al., (1988) Mol Immunol 25 (1): 7-15); solid phase direct biotin-avidin EIA (Cheung R C et al., (1990) Virology 176:546-52); and direct labeled RIA. (Moldenhauer G et al., (1990) Scand J Immunol 32:77-82). Typically, such an assay involves the use of purified antigen (e.g., PILRA such as human PILRA) bound to a solid surface or cells bearing such antigen, an unlabeled test immunoglobulin and a labeled reference immunoglobulin. Competitive inhibition can be measured by determining the amount of label bound to the solid surface or cells in the presence of the test immunoglobulin. Usually the test immunoglobulin is present in excess. Usually, when a competing antibody is present in excess, it will inhibit specific binding of a reference antibody to a common antigen by at least 50-55%, 55-60%, 60-65%, 65-70%, 70-75% or more. A competition binding assay can be configured in a large number of different formats using either labeled antigen or labeled antibody. In a common version of this assay, the antigen is immobilized on a 96-well plate. The ability of unlabeled antibodies to block the binding of labeled antibodies to the antigen is then measured using radioactive or enzyme labels. For further details see, for example, Wagener C et al., (1983) J Immunol 130:2308-2315; Wagener C et al., (1984) J Immunol Methods 68:269-274; Kuroki M et al., (1990) Cancer Res 50:4872-4879; Kuroki M et al., (1992) Immunol Invest 21:523-538; Kuroki M et al., (1992) Hybridoma 11:391-407 and Antibodies: A Laboratory Manual, Ed Harlow E & Lane D editors supra, pp. 386-389.

In some aspects, a competitive binding assay is performed using surface plasmon resonance (BIAcore®), e.g., by an ‘in tandem approach’ such as that described by Abdiche Y N et al., (2009) Analytical Biochem 386:172-180, whereby PILRA antigen is immobilized on the chip surface, for example, a CM5 sensor chip and the anti-PILRA antibodies are then run over the chip. To determine if an antibody or antigen-binding fragment thereof competitively inhibits binding of an anti-PILRA antibody described herein, the anti-PILRA antibody is first run over the chip surface to achieve saturation and then the potential, competing antibody is added. Binding of the competing antibody or antigen-binding fragment thereof can then be determined and quantified relative to a non-competing control.

In some aspects, a Fortebio Octet competitive binding is used to determine that a PILRA antibody or antigen-binding fragment thereof competitively inhibits the binding of another PILRA antibody or antigen-binding fragment thereof to PILRA.

In another aspect, provided herein are antibodies that competitively inhibit (e.g., in a dose dependent manner) an antibody or antigen-binding fragment thereof described herein (e.g., X0-X34) from binding to human PILRA, as determined using assays known to one of skill in the art or described herein (e.g., ELISA competitive assays, or suspension array or surface plasmon resonance assay). An antibody that “competitively inhibits” may also be referred to as an antibody that “competes for binding” to a reference antibody.

### Antigen Binding Fragments

In some aspects, an antigen-binding fragment of an anti-PILRA antibody described herein, such as an anti-human PILRA antibody, is provided. Exemplary antigen-binding fragments include but are not limited to Fab, Fab′, F(ab′)2, and scFv, wherein the Fab, Fab′, F(ab′)2, or scFv comprises a heavy chain variable region sequence and a light chain variable region sequence of an anti-human PILRA antibody as described herein. A Fab, Fab′, F(ab′)2, or scFv can be produced by any technique known to those of skill in the art, including, but not limited to, those discussed in Section 7.3, infra. In some aspects, an antigen-binding fragment, such as a Fab, Fab′, F(ab′)2, or scFv, further comprises a moiety that extends the half-life of the antibody in vivo. The moiety is also termed a “half-life extending moiety.” Any moiety known to those of skill in the art for extending the half-life of an antigen-binding fragment, such as a Fab, Fab′, F(ab′)2, or scFv, in vivo can be used. For example, the half-life extending moiety can include an Fc region, a polymer, an albumin, or an albumin binding protein or compound. The polymer can include a natural or synthetic, optionally substituted straight or branched chain polyalkylene, polyalkenylene, polyoxylalkylene, polysaccharide, polyethylene glycol, polypropylene glycol, polyvinyl alcohol, methoxypolyethylene glycol, lactose, amylose, dextran, glycogen, or derivative thereof. Substituents can include one or more hydroxy, methyl, or methoxy groups. In some aspects, an antigen-binding fragment, such as an Fab, Fab′, F(ab′)2, or scFv, can be modified by the addition of one or more C-terminal amino acids for attachment of the half-life extending moiety. In some aspects the half-life extending moiety is polyethylene glycol or human serum albumin. In some aspects, an antigen-binding fragment, such as a Fab, Fab′, F(ab′)2, or scFv, is fused to a Fc region.

An anti-PILRA antibody (such as an anti-human PILRA antibody) or antigen-binding fragment thereof can be fused or conjugated (e.g., covalently or noncovalently linked) to a detectable label or substance. Examples of detectable labels or substances include enzyme labels, such as, glucose oxidase; radioisotopes, such as iodine (125I, 121I), carbon (14C), sulfur (35S), tritium (3H), indium (121 In), and technetium (99Tc); luminescent labels, such as luminol; and fluorescent labels, such as fluorescein and rhodamine, and biotin. Such labeled antibodies or antigen-binding fragments thereof can be used to detect PILRA (e.g., human PILRA) protein. See, e.g., Section 7.5, infra.

### 7.3 Antibody Production

Antibodies and antigen-binding fragments thereof that immunospecifically bind to human PILRA can be produced by any method known in the art for the synthesis of antibodies and antigen-binding fragments, for example, by chemical synthesis or by recombinant expression techniques. The methods described herein employ, unless otherwise indicated, conventional techniques in molecular biology, microbiology, genetic analysis, recombinant DNA, organic chemistry, biochemistry, PCR, oligonucleotide synthesis and modification, nucleic acid hybridization, and related fields within the skill of the art. These techniques are described, for example, in the references cited herein and are fully explained in the literature. See, e.g., Sambrook J et al., (2001) Molecular Cloning: A Laboratory Manual, Cold Spring Harbor Laboratory Press, Cold Spring Harbor, NY; Ausubel F M et al., Current Protocols in Molecular Biology, John Wiley & Sons (1987 and annual updates); Current Protocols in Immunology, John Wiley & Sons (1987 and annual updates); Eckstein (ed.) (1991) Oligonucleotides and Analogues: A Practical Approach, IRL Press; Birren B et al., (eds.) (1999) Genome Analysis: A Laboratory Manual, Cold Spring Harbor Laboratory Press.

In a certain aspect, provided herein is a method of making an antibody or antigen-binding fragment which immunospecifically binds to human PILRA comprising culturing a cell or host cell described herein (e.g., a cell or a host cell comprising polynucleotides encoding an antibody or antigen-binding fragment thereof described herein). In a certain aspect, provided herein is a method of making an antibody or antigen-binding fragment thereof which immunospecifically binds to human PILRA comprising expressing (e.g., recombinantly expressing) the antibody or antigen-binding fragment thereof using a cell or host cell described herein (e.g., a cell or a host cell comprising polynucleotides encoding an antibody or antigen-binding fragment thereof described herein). In some aspects, the cell is an isolated cell. In some aspects, the encoding polynucleotides have been introduced into the cell. In some aspects, the method further comprises the step of purifying the antibody or antigen-binding fragment obtained from the cell or host cell.

Methods for producing polyclonal antibodies are known in the art (see, for example, Chapter 11 in: Short Protocols in Molecular Biology, (2002) 5th Ed., Ausubel F M et al., eds., John Wiley and Sons, New York).

Monoclonal antibodies or antigen-binding fragments thereof can be prepared using a wide variety of techniques known in the art including the use of hybridoma, recombinant, and phage display technologies, yeast-based presentation technologies, or a combination thereof. For example, monoclonal antibodies or antigen-binding fragments thereof can be produced using hybridoma techniques including those known in the art and taught, for example, in Harlow E & Lane D, Antibodies: A Laboratory Manual, (Cold Spring Harbor Laboratory Press, 2nd ed. 1988); Hammerling G J et al., in: Monoclonal Antibodies and T-Cell Hybridomas 563 681 (Elsevier, N.Y., 1981), or as described in Kohler G & Milstein C (1975) Nature 256:495. Examples of yeast-based presentation methods that can be employed to select and generate the antibodies described herein include those disclosed in, for example, WO2009/036379A2; WO2010/105256; and WO2012/009568, each of which is herein incorporated by reference in its entirety.

In some aspects, a monoclonal antibody or antigen-binding fragment is an antibody or antigen-binding fragment produced by a clonal cell (e.g., hybridoma or host cell producing a recombinant antibody or antigen-binding fragment), wherein the antibody or antigen-binding fragment immunospecifically binds to human PILRA as determined, e.g., by ELISA or other antigen-binding assays known in the art or in the Examples provided herein. In some aspects, a monoclonal antibody or antigen-binding fragment thereof can be a chimeric or a humanized antibody or antigen-binding fragment thereof. In some aspects, a monoclonal antibody or antigen-binding fragment thereof can be a Fab fragment or a F(ab′)2 fragment. Monoclonal antibodies or antigen-binding fragments thereof described herein can, for example, be made by the hybridoma method as described in Kohler G & Milstein C (1975) Nature 256:495 or can, e.g., be isolated from phage libraries using the techniques as described herein, for example. Other methods for the preparation of clonal cell lines and of monoclonal antibodies and antigen-binding fragments thereof expressed thereby are well known in the art (see, for example, Chapter 11 in: Short Protocols in Molecular Biology, (2002) 5th Ed., Ausubel F M et al., supra).

Antigen-binding fragments of antibodies described herein can be generated by any technique known to those of skill in the art. For example, Fab and F(ab′)2 fragments described herein can be produced by proteolytic cleavage of immunoglobulin molecules, using enzymes such as papain (to produce Fab fragments) or pepsin (to produce F(ab′)2 fragments). A Fab fragment corresponds to one of the two identical arms of a tetrameric antibody molecule and contains the complete light chain paired with the VH and CH1 domains of the heavy chain. A F(ab′)2 fragment contains the two antigen-binding arms of a tetrameric antibody molecule linked by disulfide bonds in the hinge region.

Further, the antibodies or antigen-binding fragments thereof described herein can also be generated using various phage display and/or yeast-based presentation methods known in the art. In phage display methods, proteins are displayed on the surface of phage particles which carry the polynucleotide sequences encoding them. In particular, DNA sequences encoding VH and VL domains are amplified from animal cDNA libraries (e.g., human or murine cDNA libraries of affected tissues). The DNA encoding the VH and VL domains are recombined together with a scFv linker by PCR and cloned into a phagemid vector. The vector is electroporated in E. coli and the E. coli is infected with helper phage. Phage used in these methods are typically filamentous phage including fd and M13, and the VH and VL domains are usually recombinantly fused to either the phage gene III or gene VIII. Phage expressing an antibody or antigen-binding fragment thereof that binds to a particular antigen can be selected or identified with antigen, e.g., using labeled antigen or antigen bound or captured to a solid surface or bead. Examples of phage display methods that can be used to make the antibodies or fragments described herein include those disclosed in Brinkman U et al., (1995) J Immunol Methods 182:41-50; Ames R S et al., (1995) J Immunol Methods 184:177-186; Kettleborough C A et al., (1994) Eur J Immunol 24:952-958; Persic L et al., (1997) Gene 187:9-18; Burton D R & Barbas C F (1994) Advan Immunol 57:191-280; PCT Application No. PCT/GB91/001134; International Publication Nos. WO 90/02809, WO 91/10737, WO 92/01047, WO 92/18619, WO 93/1 1236, WO 95/15982, WO 95/20401, and WO 97/13844; and U.S. Pat. Nos. 5,698,426, 5,223,409, 5,403,484, 5,580,717, 5,427,908, 5,750,753, 5,821,047, 5,571,698, 5,427,908, 5,516,637, 5,780,225, 5,658,727, 5,733,743, and 5,969,108.

A humanized antibody or antigen-binding fragment thereof can be selected from any class of immunoglobulins, including IgM, IgG, IgD, IgA and IgE, and any isotype, including IgG1, IgG2, IgG3 and IgG4.

### 7.3.1 Polynucleotides

In certain aspects, provided herein are polynucleotides comprising a nucleotide sequence encoding an antibody or antigen-binding fragment thereof described herein or a domain thereof (e.g., a variable light chain region and/or variable heavy chain region) that immunospecifically binds to human PILRA, and vectors, e.g., vectors comprising such polynucleotides for recombinant expression in host cells (e.g., E. coli and mammalian cells).

In particular aspects, provided herein are polynucleotides comprising nucleotide sequences encoding antibodies or antigen-binding fragments thereof, which immunospecifically bind to human PILRA and comprise an amino acid sequence as described herein, as well as antibodies or antigen-binding fragments that compete with such antibodies or antigen-binding fragments for binding to a human PILRA (e.g., in a dose-dependent manner), or which bind to the same epitope as that of such antibodies or antigen-binding fragments.

Also provided herein is a polynucleotide comprising a nucleotide sequence encoding a polypeptide comprising a sequence of one or more of SEQ ID NOs: 4-6, 10-22, 25-33, 38, 39, 50, 54, 58-60, 63-71, 78, 82-84, 89-102, and 200-208. In some aspects, an antibody or antigen-binding fragment thereof comprising the polypeptide immunospecifically binds to human PILRA.

Also provided herein are kits, vectors, or host cells comprising (i) a first polynucleotide comprising a nucleotide sequence encoding a VH polypeptide provided herein (see e.g., Table 3) and (ii) a second polynucleotide comprising a nucleotide sequence encoding a VL polypeptide provided herein (see e.g., Table 4). In a kit comprising such first and second polynucleotides, the first and second polynucleotides can be in the same vector or can be in different vectors. In a host cell comprising such first and second polynucleotides, the first and second polynucleotides can in the same vector or can be in different vectors.

In some aspects, provided herein are polynucleotides comprising a nucleotide sequence encoding three VH domain CDRs, e.g., a polypeptide containing VH CDR1, VH CDR2, and VH CDR3 of any one of antibodies described herein (e.g., see Table 1), e.g., wherein the three VH domain CDRs are in the context of a VH. In some aspects, provided herein are polynucleotides comprising a nucleotide sequence encoding three VL domain CDRs, e.g., a polypeptide containing VL CDR1, VL CDR2, and VL CDR3 of any one of antibodies described herein (e.g., see Table 2), e.g., wherein the three VL domain CDRs are in the context of a VL. In some aspects, provided herein are polynucleotides (or combinations of polynucleotides) comprising a nucleotide sequence encoding an anti-human PILRA antibody or antigen-binding fragment thereof comprising (i) three VH domain CDRs, e.g., a polypeptide containing VH CDR1, VH CDR2, and VH CDR3 of any one of antibodies described herein (e.g., see Table 1) e.g., wherein the three VH domain CDRs are in the context of a VH and (ii) three VL domain CDRs, e.g., a polypeptide containing VL CDR1, VL CDR2, and VL CDR3 of any one of antibodies described herein (e.g., see Table 2) e.g., wherein the three VL domain CDRs are in the context of a VL.

In some aspects, provided herein are polynucleotides comprising a nucleotide sequence encoding an anti-human PILRA antibody or an antigen-binding fragment thereof or a fragment thereof comprising a VH domain, e.g., containing FR1-CDR1-FR2-CDR2-FR3-CDR3-FR4, comprising an amino acid sequence described herein (e.g., see Tables 1 and 5, e.g., the VH CDRs and VH FRs of a particular antibody identified by name in the tables). In some aspects, provided herein are polynucleotides comprising a nucleotide sequence encoding an anti-human PILRA antibody or antigen-binding fragment thereof or a fragment thereof comprising a VL domain, e.g., containing FR1-CDR1-FR2-CDR2-FR3-CDR3-FR4, comprising an amino acid sequence described herein (e.g., see Tables 2 and 6, e.g., the VL CDRs and VL FRs of a particular antibody identified by name in the Tables).

In some aspects, a polynucleotide comprises a nucleic acid sequence encoding a heavy chain variable region (e.g., a VH comprising an amino acid sequence provided in Table 3) and a heavy chain constant region, e.g., a human gamma (Y) heavy chain constant region.

In some aspects, a polynucleotide comprises a nucleic acid sequence encoding a light chain variable region (e.g., a VL comprising an amino acid sequence provided in Table 4) and a light chain constant region, e.g., a human lambda or kappa light chain constant region.

Also provided herein are polynucleotides encoding an anti-human PILRA antibody or antigen-binding fragment thereof described herein or a domain thereof that are optimized, e.g., by codon/RNA optimization, replacement with heterologous signal sequences, and elimination of mRNA instability elements. Methods to generate optimized nucleic acids encoding an anti-human PILRA antibody or antigen-binding fragment thereof or a domain thereof (e.g., heavy chain, light chain, VH domain, or VL domain) for recombinant expression by introducing codon changes (e.g., a codon change that encodes the same amino acid due to the degeneracy of the genetic code) and/or eliminating inhibitory regions in the mRNA can be carried out by adapting the optimization methods described in, e.g., U.S. Pat. Nos. 5,965,726; 6,174,666; 6,291,664; 6,414,132; and 6,794,498, accordingly.

A polynucleotide encoding an antibody or antigen-binding fragment thereof described herein or a domain thereof can be generated from nucleic acid from a suitable source (e.g., a hybridoma) using methods well known in the art (e.g., PCR and other molecular cloning methods). For example, PCR amplification using synthetic primers hybridizable to the 3′ and 5′ ends of a known sequence can be performed using genomic DNA obtained from hybridoma cells producing the antibody of interest. Such PCR amplification methods can be used to obtain nucleic acids comprising the sequence encoding the light chain and/or heavy chain of an antibody or antigen-binding fragment thereof. Such PCR amplification methods can be used to obtain nucleic acids comprising the sequence encoding the variable light chain region and/or the variable heavy chain region of an antibody or antigen-binding fragment thereof. The amplified nucleic acids can be cloned into vectors for expression in host cells and for further cloning, for example, to generate chimeric and humanized antibodies or antigen-binding fragments thereof.

Polynucleotides provided herein can be, e.g., in the form of RNA or in the form of DNA. DNA includes cDNA, genomic DNA, and synthetic DNA, and DNA can be double-stranded or single-stranded. If single stranded, DNA can be the coding strand or non-coding (anti-sense) strand. In some aspects, the polynucleotide is a cDNA or a DNA lacking one more endogenous introns. In some aspects, a polynucleotide is a non-naturally occurring polynucleotide. In some aspects, a polynucleotide is recombinantly produced. In some aspects, the polynucleotides are isolated. In some aspects, the polynucleotides are substantially pure. In some aspects, a polynucleotide is purified from natural components.

### 7.3.2 Cells and Vectors

In certain aspects, provided herein are vectors (e.g., expression vectors) comprising polynucleotides comprising nucleotide sequences encoding anti-human PILRA antibodies and antigen-binding fragments thereof or a domain thereof for recombinant expression in host cells, preferably in mammalian cells. Also provided herein are cells, e.g. host cells, comprising such vectors for recombinantly expressing anti-human PILRA antibodies or antigen-binding fragments thereof described herein (e.g., human or humanized antibodies or antigen-binding fragments thereof). In some aspects, provided herein are methods for producing an antibody or antigen-binding fragments thereof described herein, comprising expressing such antibody or antigen-binding fragment thereof in a host cell.

In some aspects, recombinant expression of an antibody or antigen-binding fragment thereof or domain thereof described herein (e.g., a heavy or light chain described herein) that specifically binds to human PILRA involves construction of an expression vector containing a polynucleotide that encodes the antibody or antigen-binding fragment thereof or domain thereof. Once a polynucleotide encoding an antibody or antigen-binding fragment thereof or domain thereof (e.g., heavy or light chain variable domain) described herein has been obtained, the vector for the production of the antibody or antigen-binding fragment thereof can be produced by recombinant DNA technology using techniques well known in the art. Thus, methods for preparing a protein by expressing a polynucleotide containing an antibody or antigen-binding fragment thereof or domain thereof (e.g., light chain or heavy chain) encoding nucleotide sequence are described herein. Methods which are well known to those skilled in the art can be used to construct expression vectors containing antibody or antigen-binding fragment thereof or domain thereof (e.g., light chain or heavy chain) coding sequences and appropriate transcriptional and translational control signals. These methods include, for example, in vitro recombinant DNA techniques, synthetic techniques, and in vivo genetic recombination. Also provided are replicable vectors comprising a nucleotide sequence encoding an antibody or antigen-binding fragment thereof described herein, a heavy or light chain, a heavy or light chain variable domain, or a heavy or light chain CDR, operably linked to a promoter. Such vectors can, for example, include the nucleotide sequence encoding the constant region of the antibody or antigen-binding fragment thereof (see, e.g., International Publication Nos. WO 86/05807 and WO 89/01036; and U.S. Pat. No. 5,122,464), and variable domains of the antibody or antigen-binding fragment thereof can be cloned into such a vector for expression of the entire heavy, the entire light chain, or both the entire heavy and light chains.

An expression vector can be transferred to a cell (e.g., host cell) by conventional techniques and the resulting cells can then be cultured by conventional techniques to produce an antibody or antigen-binding fragment thereof described herein (e.g., an antibody or antigen-binding fragment thereof comprising the six CDRs, the VH, the VL, the VH and the VL, the heavy chain, the light chain, or the heavy and the light chain of X0-X34) or a domain thereof (e.g., the VH, the VL, the VH and the VL, the heavy chain, or the light chain of X0-X34). Thus, provided herein are host cells containing a polynucleotide encoding an antibody or antigen-binding fragment thereof described herein (e.g., an antibody or antigen-binding fragment thereof comprising the six CDRs, the VH, the VL, the VH and the VL, the heavy chain, the light chain, or the heavy and the light chain of X0-X34) or a domain thereof (e.g., the VH, the VL, the VH and the VL, the heavy chain, or the light chain of X0-X34), operably linked to a promoter for expression of such sequences in the host cell. In some aspects, for the expression of double-chained antibodies or antigen-binding fragments thereof, vectors encoding both the heavy and light chains, individually, can be co-expressed in the host cell for expression of the entire immunoglobulin, as detailed below. In some aspects, a host cell contains a vector comprising a polynucleotide encoding both the heavy chain and light chain of an antibody described herein (e.g., the heavy and the light chain of X0-X34), or a domain thereof (e.g., the VH and the VL of X0-X34). In some aspects, a host cell contains two different vectors, a first vector comprising a polynucleotide encoding a heavy chain or a heavy chain variable region of an antibody or antigen-binding fragment thereof described herein, and a second vector comprising a polynucleotide encoding a light chain or a light chain variable region of an antibody described herein (e.g., an antibody comprising the six CDRs of X0-X34), or a domain thereof. In some aspects, a first host cell comprises a first vector comprising a polynucleotide encoding a heavy chain or a heavy chain variable region of an antibody or antigen-binding fragment thereof described herein, and a second host cell comprises a second vector comprising a polynucleotide encoding a light chain or a light chain variable region of an antibody or antigen-binding fragment thereof described herein (e.g., an antibody or antigen-binding fragment thereof comprising the six CDRs of X0-X34). In some aspects, a heavy chain/heavy chain variable region expressed by a first cell associated with a light chain/light chain variable region of a second cell to form an human PILRA antibody or antigen-binding fragment thereof described herein (e.g., antibody or antigen-binding fragment thereof comprising the six CDRs of X0-X34). In some aspects, provided herein is a population of host cells comprising such first host cell and such second host cell.

In some aspects, provided herein is a population of vectors comprising a first vector comprising a polynucleotide encoding a light chain/light chain variable region of an anti-human PILRA antibody or antigen-binding fragment thereof described herein, and a second vector comprising a polynucleotide encoding a heavy chain/heavy chain variable region of an anti-human PILRA antibody or antigen-binding fragment thereof described herein (e.g., antibody or antigen-binding fragment thereof comprising the CDRs of X0-X34). Alternatively, a single vector can be used which encodes, and is capable of expressing, both heavy and light chain polypeptides.

A variety of host-expression vector systems can be utilized to express antibodies and antigen-binding fragments thereof described herein (e.g., an antibody or antigen-binding fragment thereof comprising the CDRs of X0-X34) (see, e.g., U.S. Pat. No. 5,807,715). Such host-expression systems represent vehicles by which the coding sequences of interest can be produced and subsequently purified, but also represent cells which can, when transformed or transfected with the appropriate nucleotide coding sequences, express an antibody or antigen-binding fragment thereof described herein in situ. These include but are not limited to microorganisms such as bacteria (e.g., E. coli and B. subtilis) transformed with recombinant bacteriophage DNA, plasmid DNA or cosmid DNA expression vectors containing antibody coding sequences; yeast (e.g., Saccharomyces Pichia) transformed with recombinant yeast expression vectors containing antibody coding sequences; insect cell systems infected with recombinant virus expression vectors (e.g., baculovirus) containing antibody coding sequences; plant cell systems (e.g., green algae such as Chlamydomonas reinhardtii) infected with recombinant virus expression vectors (e.g., cauliflower mosaic virus, CaMV; tobacco mosaic virus, TMV) or transformed with recombinant plasmid expression vectors (e.g., Ti plasmid) containing antibody coding sequences; or mammalian cell systems (e.g., COS (e.g., COS1 or COS), CHO, BHK, MDCK, HEK 293, NSO, PER.C6, VERO, CRL7030, HsS78Bst, HeLa, and NIH 3T3, HEK-293T, HepG2, SP210, R1.1, B-W, L-M, BSC1, BSC40, YB/20 and BMT10 cells) harboring recombinant expression constructs containing promoters derived from the genome of mammalian cells (e.g., metallothionein promoter) or from mammalian viruses (e.g., the adenovirus late promoter; the vaccinia virus 7.5K promoter). In some aspects, cells for expressing antibodies and antigen-binding fragments thereof described herein (e.g., an antibody or antigen-binding fragment thereof comprising the CDRs of X0-X34) are CHO cells, for example CHO cells from the CHO GS System™ (Lonza). In some aspects, cells for expressing antibodies described herein are human cells, e.g., human cell lines. In some aspects, a mammalian expression vector is pOptiVEC™ or pcDNA3.3. In some aspects, bacterial cells such as Escherichia coli, or eukaryotic cells (e.g., mammalian cells), especially for the expression of whole recombinant antibody molecule, are used for the expression of a recombinant antibody molecule. For example, mammalian cells such as Chinese hamster ovary (CHO) cells in conjunction with a vector such as the major intermediate early gene promoter element from human cytomegalovirus is an effective expression system for antibodies (Foecking M K & Hofstetter H (1986) Gene 45:101-105; and Cockett M I et al., (1990) Biotechnology 8:662-667). In some aspects, antibodies or antigen-binding fragments thereof described herein are produced by CHO cells or NSO cells.

In addition, a host cell strain can be chosen which modulates the expression of the inserted sequences, or modifies and processes the gene product in the specific fashion desired. Such modifications (e.g., glycosylation) and processing (e.g., cleavage) of protein products can contribute to the function of the protein. To this end, eukaryotic host cells which possess the cellular machinery for proper processing of the primary transcript, glycosylation, and phosphorylation of the gene product can be used. Such mammalian host cells include but are not limited to CHO, VERO, BHK, Hela, MDCK, HEK 293, NIH 3T3, W138, BT483, Hs578T, HTB2, BT20 and T47D, NSO (a murine myeloma cell line that does not endogenously produce any immunoglobulin chains), CRL7030, COS (e.g., COS1 or COS), PER.C6, VERO, HsS78Bst, HEK-293T, HepG2, SP210, R1.1, B-W, L-M, BSC1, BSC40, YB/20, BMT10 and HsS78Bst cells. In some aspects, anti-human PILRA antibodies or antigen-binding fragments thereof described herein (e.g., an antibody or antigen-binding fragment thereof comprising the CDRs of X0-X34) are produced in mammalian cells, such as CHO cells.

Once an antibody or antigen-binding fragment thereof described herein has been produced by recombinant expression, it can be purified by any method known in the art for purification of an immunoglobulin molecule, for example, by chromatography (e.g., ion exchange, affinity, particularly by affinity for the specific antigen after Protein A, and sizing column chromatography), centrifugation, differential solubility, or by any other standard technique for the purification of proteins. Further, the antibodies or antigen-binding fragments thereof described herein can be fused to heterologous polypeptide sequences described herein or otherwise known in the art to facilitate purification.

In some aspects, an antibody or antigen-binding fragment thereof described herein is isolated or purified. Generally, an isolated antibody or antigen-binding fragment thereof is one that is substantially free of other antibodies or antigen-binding fragments thereof with different antigenic specificities than the isolated antibody or antigen-binding fragment thereof. For example, in some aspects, a preparation of an antibody or antigen-binding fragment thereof described herein is substantially free of cellular material and/or chemical precursors.

### 7.4 Pharmaceutical Compositions

Provided herein are compositions comprising an anti-PILRA antibody (such as an anti-human PILRA antibody) or antigen-binding fragment thereof, as described herein. In some aspects, the antibody or antigen-binding fragment thereof having the desired degree of purity is present in a formulation comprising, e.g., a physiologically acceptable carrier, excipient or stabilizer (Remington's Pharmaceutical Sciences (1990) Mack Publishing Co., Easton, PA). Acceptable carriers, excipients, or stabilizers are nontoxic to recipients at the dosages and concentrations employed. Formulations suitable for parenteral administration include aqueous and non-aqueous, isotonic sterile injection solutions, which can comprise antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives.

In some aspects, a pharmaceutical composition comprises an anti-human PILRA antibody or antigen-binding fragment thereof as described herein, and a pharmaceutically acceptable carrier (see, e.g., Gennaro, Remington: The Science and Practice of Pharmacy with Facts and Comparisons: Drugfacts Plus, 20th ed. (2003); Ansel et al., Pharmaceutical Dosage Forms and Drug Delivery Systems, 7th ed., Lippencott Williams and Wilkins (2004); Kibbe et al., Handbook of Pharmaceutical Excipients, 3rd ed., Pharmaceutical Press (2000)). Pharmaceutical compositions described herein are, in some aspects, for use as a medicament. The compositions to be used for in vivo administration can be sterile. This is readily accomplished by filtration through, e.g., sterile filtration membranes.

A pharmaceutical composition described herein can be used to exert a biological effect(s) in vivo or in vitro. For example, a pharmaceutical composition described herein can be used to activate myeloid cells, promote differentiation of myeloid cells, inhibit binding of PILRA to one or more of its ligands, inhibit binding of soluble PILRA to T cells, and/or to cells expressing such ligands (e.g., T cells), and/or downregulate cell surface PILRA.

A pharmaceutical compositions described herein can be used to treat a disease or condition, such as a disease or condition that would be alleviated by activating myeloid cells, promoting the differentiation of myeloid cells, inhibiting the binding of PILRA to one or more of its ligands and/or to cells expressing such ligands (e.g., T cells), and/or downregulating cell surface PILRA. A pharmaceutical composition described herein can be used to treat a disease or condition in which myeloid cells are dysfunctional (e.g., hypoactive) or deficient, e.g., a disease or condition in which myeloid cell activation or differentiation is desired, or in which activation of the innate immune system is desired.

In some aspects, a pharmaceutical composition provided herein is used to treat diseases or conditions such as cancer. Examples of cancers that can be treated as provided herein include solid tumors, e.g., solid tumors in which myeloid cells (monocytes, macrophages, dendritic cells, granulocytes, neutrophils, microglia (in the CNS) or other innate immune cells) have infiltrated the tumor microenvironment. Examples of such cancers that can be treated by the pharmaceutical compositions provided herein include, but are not limited to, glioblastoma, head and neck cancer, kidney cancer (e.g., kidney clear cell cancer), pancreatic cancer, and breast cancer. Other cancers include, but are not limited to, ovarian cancer, sarcoma, colorectal cancer, lung cancer, melanoma, bladder cancer, liver cancer and uterine cancer. In some aspects, a cancer is a hematopoietic cancer, such as a leukemia, lymphoma, or myeloma. In some aspects, a cancer may be an early stage cancer or a late stage cancer. In some aspects, a cancer is a primary tumor. In some aspects, a cancer is a metastatic tumor at a second site derived from any of the above types of cancer. In some aspects, a cancer is a PILRA-positive cancer. In some aspects, a cancer is a cancer with increased PILRA (e.g. increased PILRA mRNA and/or increased PILRA protein).

In some aspects, a pharmaceutical composition provided herein is used to treat a neurodegenerative disease. In some aspects, the neurodegenerative disease is characterized by dysfunctional (e.g., hypoactive) or deficient myeloid cells, such as microglia. In some aspects, the neurodegenerative disease is an immune-mediated neurodegenerative disease. In some aspects, the neurodegenerative disease is selected from Alzheimer's disease, dementia, frontotemporal dementia (FTD), vascular dementia, mild cognitive impairment, Parkinson's disease, amyotrophic lateral sclerosis (ALS), Huntington's disease, Tauopathy disease, multiple sclerosis, immune-mediated neuropathies (such as neuropathic pain), Nasu-Hakola disease, pediatric-onset leukoencephalopathy, adult-onset leukoencephalopathy with axonal spheroids and pigmented glia (ALSP), and limbic-predominant age-related TDP43 encephalopathy (LATE).

In some aspects, a pharmaceutical composition provided herein is used to inhibit HSV-1 infection. In some aspects, a pharmaceutical composition provided herein is used to inhibit HSV-1 reoccurance.

### 7.5 Uses and Methods

In various aspects, provided herein are in vitro and in vivo methods of using anti-human PILRA antibodies or antigen-binding fragments thereof as described herein, or pharmaceutical compositions thereof as described herein. In one aspect, a method of activating a myeloid cell is provided, the method comprising exposing the cell to an anti-human PILRA antibody or antigen-binding fragment thereof, or pharmaceutical composition thereof. In another aspect, a method of promoting differentiation of a myeloid cell is provided, the method comprising exposing the cell to an anti-human PILRA antibody or antigen-binding fragment thereof, or pharmaceutical composition thereof. In another aspect, a method of inhibiting binding of PILRA to one or more of its ligands is provided, the method comprising contacting PILRA with an anti-human PILRA antibody or antigen-binding fragment thereof, or pharmaceutical composition thereof, in the presence of one or more of its ligands. Exemplary ligands include glycosylated proteins, such as those with a PTPXP, PTPXXP, PXTPXP, or PXTPXXP motif. Exemplary ligands include, e.g., COLEC12, NPDC1, CLEC4G, and PIANP, as well as the HSV-1 glycoprotein B. In certain aspects, PILRA and/or the one or more of its ligands are expressed on cells. In another aspect, a method of downregulating cell surface PILRA is provided, the method comprising exposing a cell expressing PILRA on its surface to an anti-human PILRA antibody or antigen-binding fragment thereof, or pharmaceutical composition thereof.

### 7.5.1 Therapeutic Uses and Methods

In some aspects, provided herein are methods for increasing myeloid cell activation in a subject (e.g., a human subject) in need thereof, comprising administering to the subject an anti-human PILRA antibody or antigen-binding fragment thereof as described herein, or a pharmaceutical composition thereof as described herein. In some aspects, provided herein are methods for promoting myeloid cell differentiation in a subject (e.g., a human subject) in need thereof, comprising administering to the subject an anti-human PILRA antibody or antigen-binding fragment thereof as described herein, or a pharmaceutical composition thereof as described herein.

In some aspects, methods are provided in which an anti-human PILRA antibody or antigen-binding fragment thereof as described herein, or pharmaceutical composition as described herein, is administered to a subject (e.g., a human subject) in need thereof to inhibit interaction of PILRA with one or more of its ligands (e.g., NPDC1) in the subject. In some aspects, methods are provided in which an anti-human PILRA antibody or antigen-binding fragment thereof as described herein, or pharmaceutical composition thereof as described herein, is administered to a subject (e.g., a human subject) in need thereof to downregulate cell surface PILRA in the subject.

In some aspects, the anti-human PILRA antibody or antigen-binding fragment thereof, or pharmaceutical composition thereof, is administered to achieve any two or more of the above effects.

In some aspects, provided herein are methods of treating a disease or condition that would be alleviated by activating myeloid cells, promoting the differentiation of myeloid cells, inhibiting the binding of PILRA to one or more of its ligands and/or downregulating cell surface PILRA. Such methods can comprise administering an anti-human PILRA antibody or antigen-binding fragment thereof as described herein, or a pharmaceutical composition thereof as described herein, to a patient (e.g., a human patient) in need thereof.

In some aspects, provided herein are methods of treating a disease or condition in which myeloid cells are dysfunctional (e.g., hypoactive) or deficient, e.g., a disease or condition in which myeloid cell activation or differentiation is desired, or in which activation of the innate immune system is desired. Such methods can comprise administering an anti-human PILRA antibody or antigen-binding fragment thereof as described herein, or a pharmaceutical composition thereof as described herein, to a patient (e.g., a human patient) in need thereof. A disease or condition in which myeloid cells are dysfunctional may include cancer or neurodegenerative diseases, as further described herein.

In some aspects, provided herein are methods of treating cancer. A method of treating cancer can comprise administering an anti-human PILRA antibody or antigen-binding fragment thereof as described herein, or a pharmaceutical composition thereof as described herein, to a patient (e.g., a human patient) in need thereof. In some aspects, provided herein are methods of treating cancer, wherein the cancer is a solid tumor. Solid tumors include those in which myeloid cells (monocytes, macrophages, dendritic cells, granulocytes, neutrophils, microglia (in the CNS) or other innate immune cells) have infiltrated the tumor microenvironment. Examples of such cancers that can be treated as provided herein include, but are not limited to, glioblastoma, head and neck cancer, kidney cancer (e.g., kidney clear cell cancer), pancreatic cancer, and breast cancer. Other cancers include, but are not limited to, ovarian cancer, sarcoma, colorectal cancer, lung cancer, melanoma, bladder cancer, liver cancer, and uterine cancer.

In some aspects, a cancer to be treated by the methods of the present disclosure includes, without limitation, a hematopoietic cancer, such as a leukemia, lymphoma, or myeloma. In some aspects, a cancer to be treated by the methods of the present disclosure may be an early stage cancer or a late stage cancer. In some aspects, a cancer may be a primary tumor. In some aspects, a cancer may be a metastatic tumor at a second site derived from any of the above types of cancer.

In some aspects, a cancer to be treated by the methods of the present disclosure is a PILRA-positive cancer. In some aspects, a cancer to be treated by the methods of the present invention is a cancer with increased PILRA (e.g. increased PILRA mRNA and/or increased PILRA protein).

In some aspects, a method of treating a cancer is provided, wherein the method comprises administering an anti-human PILRA antibody or antigen-binding fragment thereof, or pharmaceutical composition thereof, and wherein the method further comprises administering an antagonist of an inhibitory immune checkpoint molecule. In some aspects, the inhibitory checkpoint molecule is PD-1 (programmed cell death protein-1) or its ligand PD-L1 (programmed death ligand-1). In some aspects, an antagonist of PD-1 is an antibody to PD-1. PD-1 antibodies include, for example, OPDIVO (nivolumab), KEYTRUDA (pembrolizumab), MEDI-0680 (AMP-514; WO2012/145493), camrelizumab (SHR-1210), tislelizumab (BGB-A317), or spartalizumab (NPVPDR001, NVS240118, and PDR001). A recombinant protein composed of the extracellular domain of PD-L2 (B7-DC) used to the Fc portion of IgG1, called AMP-224, can also be used to antagonize the PD-1 receptor. In some aspects, an antagonist of PD-L1 is an antibody to PD-L1. PD-L1 antibodies include, for example, TECENTRIQ (atezolizumab), durvalumab (MEDI4736), BMS-936559 (WO2007/005874), MSB0010718C (WO2013/79174) or rHigM12B7. In some aspects, an anti-human PILRA antibody or antigen-binding fragment thereof, or pharmaceutical composition thereof, is administered in combination with radiation therapy and/or a chemotherapeutic agent.

In some aspects, provided herein are methods of inhibiting HSV-1 infection and/or HSV-1 reoccurance. A method of inhibiting HSV-1 infection and/or reoccurance can comprise administering an anti-human PILRA antibody or antigen-binding fragment thereof as described herein, or a pharmaceutical composition thereof as described herein, to a patient (e.g., a human patient) in need thereof.

In some aspects, provided herein are methods of treating a neurodegenerative disease. A method of treating a neurodegenerative disease can comprise administering an anti-human PILRA antibody or antigen-binding fragment thereof as described herein, or a pharmaceutical composition thereof as described herein, to a patient (e.g., a human patient) in need thereof. In some aspects, the neurodegenerative disease is characterized by dysfunctional (e.g., hypoactive) or deficient myeloid cells, such as microglia. Microglia are innate immune cells that reside specifically in the brain and that function as macrophages, clearing debris and dead neurons through the process of phagocytosis and providing other supportive functions for maintaining brain health. In some aspects, the patient has symptoms of a neurodegenerative disease, and an anti-human PILRA antibody or antigen-binding fragment thereof as described herein, or a pharmaceutical composition thereof as described herein, is administered to treat the neurodegenerative disease. In some aspects, the patient is at risk of developing a neurodegenerative disease, and the anti-human PILRA antibody, antigen-binding fragment, or pharmaceutical composition is administered to reduce risk, slow onset, or prevent the neurodegenerative disease.

In some aspects, the neurodegenerative disease is an immune-mediated neurodegenerative disease. In some aspects, the neurodegenerative disease is selected from Alzheimer's disease, dementia, frontotemporal dementia (FTD), vascular dementia, mild cognitive impairment, Parkinson's disease, amyotrophic lateral sclerosis (ALS), Huntington's disease, Tauopathy disease, multiple sclerosis (MS), immune-mediated neuropathies (such as neuropathic pain), Nasu-Hakola disease, pediatric-onset leukoencephalopathy, adult-onset leukoencephalopathy with axonal spheroids and pigmented glia (ALSP), and limbic-predominant age-related TDP43 encephalopathy (LATE).

### Alzheimer's Disease

Alzheimer's disease (AD) is the most common form of dementia. There is no cure for the disease, which worsens as it progresses, and eventually leads to death. Most often, AD is diagnosed in people over 65 years of age. However, the less-prevalent early-onset Alzheimer's can occur much earlier. Common symptoms of Alzheimer's disease include, behavioral symptoms, such as difficulty in remembering recent events; cognitive symptoms, confusion, irritability and aggression, mood swings, trouble with language, and long-term memory loss. As the disease progresses bodily functions are lost, ultimately leading to death. Alzheimer's disease develops for an unknown and variable amount of time before becoming fully apparent, and it can progress undiagnosed for years.

In some aspects, administering an anti-human PILRA antibody or antigen-binding fragment thereof as provided herein, or a pharmaceutical composition thereof as provided herein, can prevent, reduce the risk, and/or treat Alzheimer's disease in an individual. In some aspects, the individual has early-onset Alzheimer's disease. In some aspects, the individual has late-onset Alzheimer's disease. In some aspects, the individual is experiencing the prodromal stage of

Alzheimer's disease. In some aspects, the individual has mild Alzheimer's disease. In some aspects, administering the anti-human PILRA antibody, antigen-binding fragment or pharmaceutical composition may modulate one or more PILRA activities in an individual having Alzheimer's disease.

### Dementia

Dementia is a non-specific syndrome (i.e., a set of signs and symptoms) that presents as a serious loss of global cognitive ability in a previously unimpaired person, beyond what might be expected from normal ageing. Dementia may be static as the result of a unique global brain injury. Alternatively, dementia may be progressive, resulting in long-term decline due to damage or disease in the body. While dementia is much more common in the geriatric population, it can also occur before the age of 65. Cognitive areas affected by dementia include, without limitation, memory, attention span, language, and problem solving. Generally, symptoms must be present for at least six months to before an individual is diagnosed with dementia.

Exemplary forms of dementia include, without limitation, frontotemporal dementia, Alzheimer's disease, vascular dementia, semantic dementia, and dementia with Lewy bodies.

In some aspects, administering an anti-human PILRA antibody or antigen-binding fragment thereof as provided herein, or a pharmaceutical composition thereof as provided herein, can prevent, reduce the risk, and/or treat dementia. In some aspects, administering the anti-human PILRA antibody, antigen-binding fragment or pharmaceutical composition may modulate one or more PILRA activities in an individual having dementia.

### Frontotemporal Dementia

Frontotemporal dementia (FTD) is a condition resulting from the progressive deterioration of the frontal lobe of the brain. Over time, the degeneration may advance to the temporal lobe. Second only to Alzheimer's disease (AD) in prevalence, FTD accounts for 20% of pre-senile dementia cases. The clinical features of FTD include memory deficits, behavioral abnormalities, personality changes, and language impairments (Cruts, M. & Van Broeckhoven, C., Trends Genet. 24:186-194 (2008); Neary, D., et al., Neurology 51:1546-1554 (1998); Ratnavalli, E., Brayne, C., Dawson, K. & Hodges, J. R., Neurology 58:1615-1621 (2002)).

A substantial portion of FTD cases are inherited in an autosomal dominant fashion, but even in one family, symptoms can span a spectrum from FTD with behavioral disturbances, to Primary Progressive Aphasia, to Cortico-Basal Ganglionic Degeneration. FTD, like most neurodegenerative diseases, can be characterized by the pathological presence of specific protein aggregates in the diseased brain. Historically, the first descriptions of FTD recognized the presence of intraneuronal accumulations of hyperphosphorylated Tau protein in neurofibrillary tangles or Pick bodies. A causal role for the microtubule associated protein Tau was supported by the identification of mutations in the gene encoding the Tau protein in several families (Hutton, M., et al., Nature 393:702-705 (1998). However, the majority of FTD brains show no accumulation of hyperphosphorylated Tau but do exhibit immunoreactivity to ubiquitin (Ub) and TAR DNA binding protein (TDP43) (Neumann, M., et al., Arch. Neurol. 64:1388-1394 (2007)). A majority of those FTD cases with Ub inclusions (FTD-U) were shown to carry mutations in the Progranulin gene.

In some aspects, administering an anti-human PILRA antibody or antigen-binding fragment thereof as provided herein, or a pharmaceutical composition thereof as provided herein, of the present disclosure, can prevent, reduce the risk, and/or treat FTD. In some aspects, administering the anti-human PILRA antibody, antigen-binding fragment or pharmaceutical composition may modulate one or more PILRA activities in an individual having FTD.

### Parkinson's Disease

Parkinson's disease, which may be referred to as idiopathic or primary parkinsonism, hypokinetic rigid syndrome (HRS), or paralysis agitans, is a neurodegenerative brain disorder that affects motor system control. The progressive death of dopamine-producing cells in the brain leads to the major symptoms of Parkinson's. Most often, Parkinson's disease is diagnosed in people over 50 years of age. Parkinson's disease is idiopathic (having no known cause) in most people. However, genetic factors also play a role in the disease.

Symptoms of Parkinson's disease include, without limitation, tremors of the hands, arms, legs, jaw, and face, muscle rigidity in the limbs and trunk, slowness of movement (bradykinesia), postural instability, difficulty walking, neuropsychiatric problems, changes in speech or behavior, depression, anxiety, pain, psychosis, dementia, hallucinations, and sleep problems.

In some aspects, administering an anti-human PILRA antibody or antigen-binding fragment thereof as provided herein, or a pharmaceutical composition thereof as provided herein, can prevent, reduce the risk, and/or treat Parkinson's disease. In some aspects, administering the anti-human PILRA antibody, antigen-binding fragment or pharmaceutical composition may modulate one or more PILRA activities in an individual having Parkinson's disease.

### Amyotrophic Lateral Sclerosis (ALS)

As used herein, amyotrophic lateral sclerosis (ALS) or, motor neuron disease or, Lou Gehrig's disease are used interchangeably and refer to a debilitating disease with varied etiology characterized by rapidly progressive weakness, muscle atrophy and fasciculations, muscle spasticity, difficulty speaking (dysarthria), difficulty swallowing (dysphagia), and difficulty breathing (dyspnea).

In some aspects, administering an anti-human PILRA antibody or antigen-binding fragment thereof as provided herein, or a pharmaceutical composition thereof as provided herein, can prevent, reduce the risk, and/or treat ALS. In some aspects, administering the anti-human PILRA antibody, antigen-binding fragment or pharmaceutical composition may modulate one or more PILRA activities in an individual having amyotrophic lateral sclerosis.

### Huntington's Disease

Huntington's disease (HD) is an inherited neurodegenerative disease caused by an autosomal dominant mutation in the Huntingtin gene (HTT). Expansion of a cytokine-adenine-guanine (CAG) triplet repeat within the Huntingtin gene results in production of a mutant form of the Huntingtin protein (Htt) encoded by the gene. This mutant Huntingtin protein (mHtt) is toxic and contributes to neuronal death. Symptoms of Huntington's disease most commonly appear between the ages of 35 and 44, although they can appear at any age.

Symptoms of Huntington's disease, include, without limitation, motor control problems, jerky, random movements (chorea), abnormal eye movements, impaired balance, seizures, difficulty chewing, difficulty swallowing, cognitive problems, altered speech, memory deficits, thinking difficulties, insomnia, fatigue, dementia, changes in personality, depression, anxiety, and compulsive behavior.

In some aspects, administering an anti-human PILRA antibody or antigen-binding fragment thereof as provided herein, or a pharmaceutical composition thereof as provided herein, can prevent, reduce the risk, and/or treat Huntington's disease (HD). In some aspects, administering the anti-human PILRA antibody, antigen-binding fragment or pharmaceutical composition may modulate one or more PILRA activities in an individual having Huntington's disease.

### Tauopathy Disease

Tauopathy diseases, or Tauopathies, are a class of neurodegenerative disease caused by aggregation of the microtubule-associated protein tau within the brain. Alzheimer's disease (AD) is the most well-known tauopathy disease and involves an accumulation of tau protein within neurons in the form of insoluble neurofibrillary tangles (NFTs). Other tauopathy diseases and disorders include progressive supranuclear palsy, dementia pugilistica (chromic traumatic encephalopathy), frontotemporal dementia and parkinsonism linked to chromosome 17, Lytico-Bodig disease (Parkinson-dementia complex of Guam), Tangle-predominant dementia, Ganglioglioma and gangliocytoma, Meningioangiomatosis, Subacute sclerosing panencephalitis, lead encephalopathy, tuberous sclerosis, Hallervorden-Spatz disease, lipofuscinosis, Pick's disease, corticobasal degeneration, Argyrophilic grain disease (AGD), Huntington's disease, and frontotemporal lobar degeneration.

In some aspects, administering an anti-human PILRA antibody or antigen-binding fragment thereof as provided herein, or a pharmaceutical composition thereof as provided herein, can prevent, reduce the risk, and/or treat a tauopathy disease. In some aspects, administering the anti-human PILRA antibody, antigen-binding fragment or pharmaceutical composition may modulate one or more PILRA activities in an individual having a tauopathy disease.

### Multiple Sclerosis

Multiple sclerosis (MS) can also be referred to as disseminated sclerosis or encephalomyelitis disseminata. MS is an inflammatory disease in which the fatty myelin sheaths around the axons of the brain and spinal cord are damaged, leading to demyelination and scarring as well as a broad spectrum of signs and symptoms. MS affects the ability of nerve cells in the brain and spinal cord to communicate with each other effectively. Nerve cells communicate by sending electrical signals called action potentials down long fibers called axons, which are contained within an insulating substance called myelin. In MS, the body's own immune system attacks and damages the myelin. When myelin is lost, the axons can no longer effectively conduct signals. MS onset usually occurs in young adults, and is more common in women.

Symptoms of MS include, without limitation, changes in sensation, such as loss of sensitivity or tingling; pricking or numbness, such as hypoesthesia and paresthesia; muscle weakness; clonus; muscle spasms; difficulty in moving; difficulties with coordination and balance, such as ataxia; problems in speech, such as dysarthria, or in swallowing, such as dysphagia; visual problems, such as nystagmus, optic neuritis including phosphenes, and diplopia; fatigue; acute or chronic pain; and bladder and bowel difficulties; cognitive impairment of varying degrees; emotional symptoms of depression or unstable mood; Uhthoff's phenomenon, which is an exacerbation of extant symptoms due to an exposure to higher than usual ambient temperatures; and Lhermitte's sign, which is an electrical sensation that runs down the back when bending the neck.

In some aspects, administering an anti-human PILRA antibody or antigen-binding fragment thereof as provided herein, or a pharmaceutical composition thereof as provided herein, can prevent, reduce the risk, and/or treat MS. In some aspects, administering the anti-human PILRA antibody, antigen-binding fragment or pharmaceutical composition may modulate one or more PILRA activities in an individual having MS.

### Administration and Dosing

An anti-human PILRA antibody or antigen-binding fragment thereof as provided herein, or a pharmaceutical composition thereof as provided herein, can be administered by any suitable means, including parenteral, intrapulmonary, intranasal, intratumoral, intralesional administration, intracerobrospinal, intracranial, intraspinal, intrasynovial, intrathecal, oral, topical, or inhalation routes. Parenteral infusions include intramuscular, intravenous administration as a bolus or by continuous infusion over a period of time, intraarterial, intra-articular, intraperitoneal, or subcutaneous administration. In some aspects, the administration is intravenous administration. In some aspects, the administration is subcutaneous.

The appropriate dosage and dosing regimen of an anti-human PILRA antibody or antigen-binding fragment thereof as provided herein, or a pharmaceutical composition thereof as provided herein, when used alone or in combination with one or more other additional therapeutic agents, will depend on the disease to be treated, the severity and course of the disease, the route of administration and other factors.

In some aspects, provided herein is an antibody or antigen-binding fragment thereof or pharmaceutical composition provided herein for use as a medicament.

In some aspects, provided herein is an antibody or antigen-binding fragment thereof or pharmaceutical composition provided herein, for use in a method for the treatment of cancer. In some aspects, provided herein is an antibody or antigen-binding fragment thereof or pharmaceutical composition provided herein, for use in a method for the treatment of cancer in a subject, comprising administering to the subject an effective amount of an antibody or antigen-binding fragment thereof or pharmaceutical composition provided herein.

In some aspects, provided herein is an antibody or antigen-binding fragment thereof or pharmaceutical composition provided herein, for use in a method for the treatment of a neurodegenerative disease. In some aspects, provided herein is an antibody or antigen-binding fragment thereof or pharmaceutical composition provided herein, for use in a method for the treatment of a neurodegenerative disease in a subject, comprising administering to the subject an effective amount of an antibody or antigen-binding fragment thereof or pharmaceutical composition provided herein.

### 7.5.2 Detection and Diagnostic Uses

An anti-human PILRA antibody or antigen-binding fragment thereof described herein (see, e.g., Section 7.2) can be used to assay PILRA protein (e.g., human PILRA protein) levels in a biological sample using classical methods known to those of skill in the art, including immunoassays, such as the enzyme linked immunosorbent assay (ELISA), immunoprecipitation, or Western blotting. Suitable antibody assay labels are known in the art and include enzyme labels, such as, glucose oxidase; radioisotopes, such as iodine (125I, 121I), carbon (14C), sulfur (35S), tritium (3H), indium (121 In), and technetium (99Tc); luminescent labels, such as luminol; and fluorescent labels, such as fluorescein and rhodamine, and biotin. Such labels can be used to label an antibody or antigen-binding fragment thereof described herein. Alternatively, a second antibody or antigen-binding fragment thereof that recognizes an anti-human PILRA antibody or antigen-binding fragment thereof described herein can be labeled and used in combination with an anti-human PILRA antibody or antigen-binding fragment thereof to detect PILRA protein (e.g., human PILRA protein) levels.

Assaying for the expression level of PILRA protein (e.g., human PILRA protein) is intended to include qualitatively or quantitatively measuring or estimating the level of a PILRA protein (e.g., human PILRA protein) in a first biological sample either directly (e.g., by determining or estimating absolute protein level) or relatively (e.g., by comparing to the disease associated protein level in a second biological sample). PILRA protein (e.g., human PILRA protein) expression level in the first biological sample can be measured or estimated and compared to a standard PILRA protein (e.g., human PILRA protein) level, the standard being taken from a second biological sample obtained from an individual not having the disorder or being determined by averaging levels from a population of individuals not having the disorder. As will be appreciated in the art, once the “standard” PILRA protein (e.g., human PILRA protein) level is known, it can be used repeatedly as a standard for comparison.

As used herein, the term “biological sample” refers to any biological sample obtained from a subject, cell line, tissue, or other source of cells potentially expressing PILRA protein (e.g., human PILRA protein). Methods for obtaining tissue biopsies and body fluids from animals (e.g., humans) are well known in the art.

An anti-human PILRA antibody described herein can be used for prognostic, diagnostic, monitoring and screening applications, including in vitro and in vivo applications well known and standard to the skilled artisan and based on the present description. Prognostic, diagnostic, monitoring and screening assays and kits for in vitro assessment and evaluation of immune system status and/or immune response may be utilized to predict, diagnose and monitor to evaluate patient samples including those known to have or suspected of having an immune system-dysfunction, cancer or neurodegenerative disease, such as Alzheimer's disease.

Anti-human PILRA antibodies and antigen-binding fragments thereof described herein can carry a detectable or functional label. When fluorescence labels are used, currently available microscopy and fluorescence-activated cell sorter analysis (FACS) or combination of both methods procedures known in the art may be utilized to identify and to quantitate the specific binding members. Anti-human PILRA antibodies or antigen-binding fragments thereof described herein can carry a fluorescence label. Exemplary fluorescence labels include, for example, reactive and conjugated probes, e.g., Aminocoumarin, Fluorescein and Texas red, Alexa Fluor dyes, Cy dyes and DyLight dyes. An anti-human PILRA antibody can carry a radioactive label, such as the isotopes 3H, 14C, 32P, 35S, 36Cl, 51Cr, 57Co, 58Co, 59Fe, 67Cu, 90Y, 99Tc, 111 In, 117Lu, 121I, 124I, 125I, 131I, 198Au, 211At, 213Bi, 225Ac and 186Re. When radioactive labels are used, currently available counting procedures known in the art may be utilized to identify and quantitate the specific binding of anti-human PILRA antibody or antigen-binding fragment to PILRA protein (e.g., human PILRA protein). In the instance where the label is an enzyme, detection may be accomplished by any of the presently utilized colorimetric, spectrophotometric, fluorospectrophotometric, amperometric or gasometric techniques as known in the art. This can be achieved by contacting a sample or a control sample with an anti-human PILRA antibody or antigen-binding fragment thereof under conditions that allow for the formation of a complex between the antibody or antigen-binding fragment thereof and PILRA protein (e.g., human PILRA protein). Any complexes formed between the antibody or antigen-binding fragment thereof and PILRA protein (e.g., human PILRA protein) are detected and compared in the sample and the control. In light of the specific binding of the antibodies or antigen-binding fragments thereof described herein to human PILRA, the antibodies or antigen-binding fragments thereof can be used to specifically detect PILRA protein (e.g., human PILRA protein) expression on the surface of cells. The antibodies or antigen-binding fragments thereof described herein can also be used to purify PILRA protein (e.g., human PILRA protein) via immunoaffinity purification.

Also included herein is an assay system which may be prepared in the form of a test kit for the quantitative analysis of the extent of the presence of PILRA protein (e.g., human PILRA protein). The system or test kit may comprise a labeled component, e.g., a labeled antibody or antigen-binding fragment, and one or more additional immunochemical reagents. See, e.g., Section 7.6 below for more on kits.

In some aspects, methods for in vitro detection of PILRA protein (e.g., human PILRA protein) in a sample, comprising contacting said sample with an antibody or antigen-binding fragment thereof, are provided herein. In some aspects, provided herein is the use of an antibody or antigen-binding fragment thereof provided herein, for in vitro detection of PILRA protein (e.g., human PILRA protein) in a sample. In some aspects, provided herein is an antibody or antigen-binding fragment thereof or composition provided herein for use in the detection of PILRA protein (e.g., human PILRA protein) in a subject or a sample obtained from a subject. In some aspects, provided herein is an antibody or antigen-binding fragment thereof provided herein for use as a diagnostic. In some aspects, the antibody comprises a detectable label.

### 7.6 Kits

Provided herein are kits comprising one or more antibodies or antigen-binding fragments thereof described herein. In some aspects, provided herein is a pharmaceutical pack or kit comprising one or more containers filled with one or more of the ingredients of the pharmaceutical compositions described herein, such as one or more antibodies or antigen-binding fragments thereof provided herein. Optionally associated with such container(s) can be a notice in the form prescribed by a governmental agency regulating the manufacture, use, or sale of pharmaceuticals or biological products, which notice reflects approval by the agency of manufacture, use or sale for human administration.

Also provided herein are kits that can be used in detection methods. In some aspects, a kit comprises an antibody or antigen-binding fragment thereof described herein, preferably a purified antibody or antigen-binding fragment thereof, in one or more containers. In some aspects, kits described herein contain a substantially isolated PILRA protein (e.g., human PILRA protein) that can be used as a control. In some aspects, the kits described herein further comprise a control antibody or antigen-binding fragment thereof which does not react with PILRA protein (e.g., human PILRA protein). In some aspects, kits described herein contain one or more elements for detecting the binding of an antibody or antigen-binding fragment thereof to PILRA protein (e.g., human PILRA protein) (e.g., the antibody or antigen-binding fragment thereof can be conjugated to a detectable substrate such as a fluorescent compound, an enzymatic substrate, a radioactive compound or a luminescent compound, or a second antibody or antigen-binding fragment thereof which recognizes the first antibody or antigen-binding fragment thereof can be conjugated to a detectable substrate). In some aspects, a kit provided herein can include a recombinantly produced or chemically synthesized PILRA protein (e.g., human PILRA protein). The PILRA protein (e.g., human PILRA protein) provided in the kit can also be attached to a solid support. In some aspects, the detecting means of the above described kit includes a solid support to which a PILRA protein (e.g., human PILRA protein) is attached. Such a kit can also include a non-attached reporter-labeled anti-human antibody or antigen-binding fragment thereof or anti-mouse/rat antibody or antigen-binding fragment thereof. In this aspect, binding of the antibody or antigen-binding fragment thereof to the PILRA protein (e.g., human PILRA protein) can be detected by binding of the said reporter-labeled antibody or antigen-binding fragment thereof.

### 8. EXAMPLES

The examples in this Section (i.e., Section 8) are offered by way of illustration, and not by way of limitation.

### Example 1: Generation of Anti-PILRA Antibodies X0 (PA-29), X29 (PA-43), and X34 (A6)

Human and cynomolgus PILRA and PILRB polypeptides were used to generate and characterize anti-PILRA antibodies that were produced. These polypeptides were generated by creating nucleic acids that encoded the amino acid sequence of human PILRA (SEQ ID NO:1), cynomolgus PILRA (SEQ ID NO:2), human PILRB (SEQ ID NO:105), and cynomolgus PILRB (SEQ ID NO:121). The nucleic acids were each cloned into a mammalian expression vector containing nucleic acids encoding a heterologous signal peptide. Proteins were expressed from these vectors in HEK 293 cells and purified using standard methods.

Antibody phage display against human PILRA (SEQ ID NO:1) and cynomolgus PILRA (SEQ ID NO:2) with deselection to human PILRB (SEQ ID NO:105) was used to identify PILRA-specific antibodies from a naïve library. Methods for performing antibody phage display are described above, e.g., in Section 7.3. Antibodies X0 (PA-29), X29 (PA-43), and X34 (A6) were produced from this method.

Parental antibody X0 (PA-29) was screened for antagonist properties (e.g., using the methods described in the Examples 5-9 below), and X0 (PA-29) was selected for screening based on its characteristics for binding to human and cynomolgus PILRA, but not binding to human PILRB.

As described below, the anti-PILRA antibodies were selected based on their ability to bind to human PILRA and cynomolgus PILRA and block binding of human PILRA to one or more of its ligands (e.g., NPDC1).

### Example 2: Affinity Maturation of Anti-PILRA Antibodies

Affinity maturation was performed on the sequence of anti-PILRA antibody X0 (PA-29) to identify antibodies with stronger binding to human and cynomolgus PILRA. Briefly, residues in heavy and light chain CDRs were randomized to generate CDR mutagenesis libraries. The CDR mutagenesis libraries were subjected to three or four rounds of selection using both human and cynomolgus PILRA proteins (as described in Example 1) to enrich mutants with improved binding to both human and cynomolgus PILRA. 69 individual mutants were screened and those with improved binding to human PILRA and cross-reactivity to cynomolgus PILRA were further characterized as described in the Examples below.

### Example 3: Molecular Cloning of Anti-PILRA Antibodies

Certain anti-PILRA affinity-matured antibodies described in Example 2 were selected for sequencing. The amino acid sequences of the heavy chain CDR sequences and light chain CDR sequences of the antibodies of the present disclosure are provided at Tables 1 and 2, respectively. The amino acid sequences of the heavy chain variable region and light chain variable region of the antibodies are set forth in Tables 3 and 4, respectively.

### Example 4: Production of Recombinant Anti-PILRA Antibodies

For the antibodies described in Example 3, anti-PILRA antibody heavy and light variable gene regions were synthesized and subcloned into mammalian expression vectors encoding human IgG4 S228P (SP) or IgG1 LALAPS and IgK, or mouse IgG1 and mouse IgK to produce recombinant antibodies in Expi293 or ExpiCHO cells (Invitrogen) using standard procedures. Supernatants were purified using protein A chromatography, and the antibody concentrations were determined by absorbance at 280 nm measurements.

### Example 5: Characterization of Binding Kinetics and Cross-Reactivity of Anti-PILRA Antibodies

Binding kinetics of human anti-PILRA-IgG4 antibodies were evaluated by surface plasmon resonance (SPR) using a Carterra LSA instrument (Carterra, Salt Lake City, UT). Antibodies were immobilized in duplicate arrays onto an HC30M sensor chip (Carterra) by amine coupling according to the manufacturer's protocol. After priming the immobilized antibodies with HBS-EP+ (Teknova, Hollister, CA, #8022) running buffer with 0.5 mg/mL BSA (MP Biomedicals LLC, #820451), the immobilized antibodies were tested for their ability to bind to recombinant human PILRA (SEQ ID NO:1) and cynomolgus PILRA (SEQ ID NO:120) and to human PILRB (SEQ ID NO:105) and cynomolgus PILRB (SEQ ID NO:121).

For binding kinetics analysis, human and cynomolgus PILRA and PILRB analytes were diluted in running buffer to 440 to 470 nM, and diluted 3-fold serially five times, to 1.8 to 1.9 nM, and injected for five minutes over the entire chip using the single channel flow cell to measure association of binding to the immobilized antibodies. Dissociation of the analytes was measured for five minutes, followed by regeneration of the chip with two 30-second injections of 10 mM glycine pH 2.0. The calculated K_{D} values are summarized in Table 8 below.

For the human PILRB interactions, the binding was too weak and heterogenous for kinetic constants to be calculated in this assay. Therefore, steady-state affinities were estimated based on the binding level of each analyte concentration at the end of the injections. These data were analyzed using the NextGenKIT high-throughput kinetics analysis software from Carterra, and are indicated in parentheses in Table 8.

A subset of these antibodies was further evaluated using the Biacore T200 (Cytiva). Anti-PILRA antibodies were diluted to 10 μg/mL and captured using an anti-Fab (Cytiva, #28958325) surface on a CM4 chip, that was prepared by amine coupling according to the manufacturer's protocol. Human PILRA and cynomolgus PILRB were diluted in running buffer to 130 nM, and diluted 4-fold serially, twice, to 8 nM. Human PILRB was similarly diluted from 260 nM to 16 nM. These analytes were injected and allowed 2 minutes of association, followed by dissociation for 5 minutes.

Data were analyzed using Biacore evaluation software to generate kinetic constants. The equilibrium dissociation constants (K_{D}) were calculated from the fitted association and dissociation rate constants (k-on and k-off) for each of the anti-PILRA antibodies. These Kn values are indicated with asterisks in Table 8 below.

TABLE 8
Binding affinities of anti-PILRA antibodies determined by SPR

| | K_{D} (nM) human | | | |
|---|---|---|---|---|
| Antibody | | PILRB | K_{D} (nM) cyno | |
| Name | PILRA | (Steady state fit) | PILRA | PILRB |
| X0 (PA-29) | 233 | (No binding) | 17 | 40 |
| | 294* | No binding* | | 144* |
| X1 | 2.5 | (39) | 1.6 | 1.1 |
| | 1.6* | 128* | | 1.1* |
| X2 | 3.5 | (53) | 1.5 | 1.1 |
| | 3.1* | 157* | | 1.7* |
| X4 | 12 | (138) | 0.2 | 0.3 |
| X5 | 2.4 | (35) | 0.1 | 0.1 |
| X6 | 3.1 | (35) | 0.1 | 0.3 |
| X7 | 3.8 | (77) | 0.4 | 0.5 |
| X9 | 2.8 | (39) | 0.1 | 0.2 |
| X30 | 3.3 | (43) | 0.1 | 0.3 |
| X31 | 2.2 | (38) | 0.1 | 0.3 |
| X10 | 3.2 | (68) | 0.3 | 0.6 |
| X11 | 4.8 | (80) | 0.3 | 0.5 |
| | 3.4* | 142* | | 0.4* |
| X12 | 1.0 | (12) | 0.2 | 0.3 |
| | 0.3* | 28* | | 0.1* |
| X13 | 1.1 | (18) | 0.3 | 0.3 |
| | 0.2* | 9.1* | | 0.1* |
| X14 | 1.1 | (4) | 0.1 | 0.2 |
| X15 | 0.1 | (2) | 0.1 | 0.1 |
| X16 | 0.5 | (2) | 0.1 | 0.2 |
| X17 | 0.4 | (2) | 0.1 | 0.3 |
| X18 | 1.3 | (4) | 0.2 | 0.3 |
| X32 | 0.1 | (2) | 0.1 | 0.1 |
| X33 | 0.6 | (2) | 0.1 | 0.1 |
| X19 | 0.3 | (2) | 0.1 | 0.1 |
| X20 | 0.5 | (2) | 0.1 | 0.1 |
| X21 | 1.8 | (43) | 0.1 | 0.2 |
| | 1.9* | Too weak to fit | | 0.2* |
| X22 | 2.2 | (54) | 0.2 | 0.3 |
| | 6.2* | 14* | | Not tested* |
| X23 | 3.9 | (35) | 0.3 | 0.7 |
| X24 | 1.7 | (16) | 0.2 | 0.6 |
| X25 | 7.0 | (211) | 0.3 | 0.5 |
| X26 | 8.1 | (199) | 0.3 | 0.6 |
| X27 | 13 | (87) | 0.5 | 1.1 |
| X28 | 3.8 | (33) | 0.3 | 0.6 |

Values in parentheses were estimated using steady state fits.
*Values generated using Biacore T200.

Evaluation by SPR of binding by purified antibodies to human and cynomolgus PILRA and PILRB showed that all of the affinity-matured antibodies described herein were cross-reactive to both human and cynomolgus PILRA and PILRB to a certain extent. The binding affinities of the engineered variants ranged from approximately 0.2 to 12 nM for human PILRA, compared to a binding affinity of over 200 nM for the parental X0 (PA-29) antibody. The binding affinities to cynomolgus PILRA and PILRB of the engineered variants were comparable to each other and sometimes higher than binding to human PILRA, ranging from approximately 0.1 to 2 nM. This contrasted with the parental X0 (PA-29) antibody, which bound to cynomolgus PILRA and PILRB with affinities of approximately 20 and 40 nM, respectively. All antibodies except the parental X0 (PA-29) antibody were cross-reactive to human PILRB at lower affinities, ranging from approximately 2 to 200 nM. Binding of human PILRB to the parental X0 (PA-29) antibody was not observed in these assays. As such, while the affinity-matured antibodies have a higher affinity for PILRB than the parental X0 (PA-29) antibody (where no binding was observed), the affinity-matured antibodies still preferentially and specifically bind human PILRA over human PILRB (as described above).

### Example 6: Anti-PILRA Antibodies Binding to Cell Surface PILRA in Human and Cynomolgus Monocytes

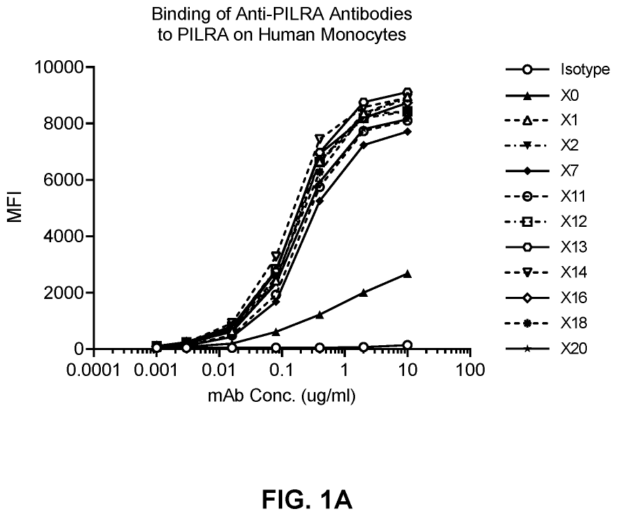

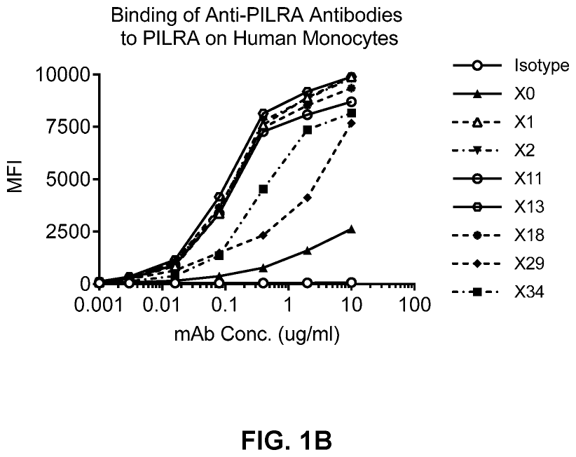

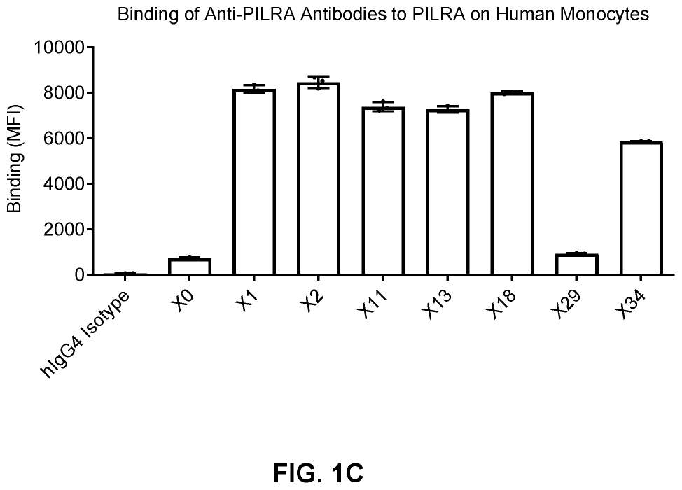

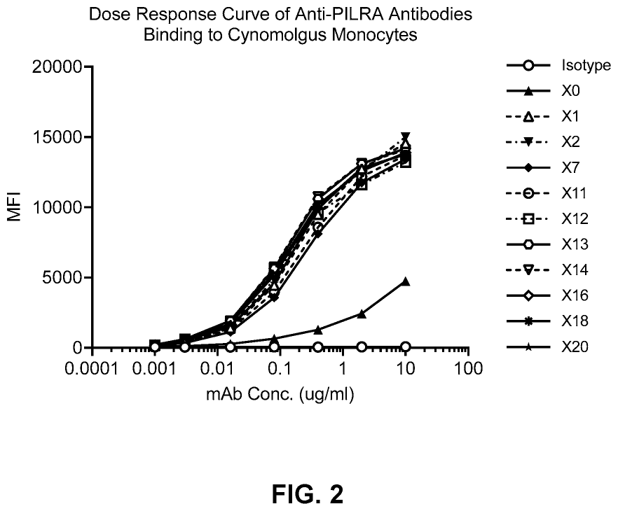

Anti-human PILRA antibodies X0 (PA-29) and its affinity matured variants were tested for their abilities to bind cell surface PILRA on human and cynomolgus monocytes. CD14+ monocytes were isolated from healthy human donors using a RosetteSep Human Monocyte Enrichment Cocktail kit (StemCell). Cynomolgus white blood cells were collected from cynomolgus whole blood. In both human and cynomolgus samples, the Fc receptor on the tested monocytes was blocked and the monocytes were treated with Alexa Fluor 647-conjugated PILRA antibodies for 30 minutes on ice. A dose response curve of antibodies X0 (PA-29), X1, X2, X7, X11, X12, X13, X14, X16, X18, and X20 was tested for binding to cell surface PILRA in human and cynomolgus monocytes, and antibodies bound to PILRA in a dose response manner. See FIGS. 1A and 2. In another sample of human donor monocytes, a dose response curve of antibodies X0 (PA-29), X1, X2, X11, X13, X18, X29 (PA-43), and X34 (A6) was tested for binding to cell surface PILRA in human and cynomolgus monocytes, and antibodies bound to PILRA in a dose response manner. See FIG. 1B. FIG. 1C shows the binding of antibodies X0 (PA-29), X1, X2, X11, X13, X18, X29 (PA-43), and X34 (A6) (1 μg/mL) to PILRA on human monocytes, with X1, X2, X11, X13, X18, and X34 (A6) demonstrating the highest binding to human PILRA.

### Example 7: Anti-PILRA Antibodies Can Decrease Binding of NPDC1 to PILRA-Expressing Cells

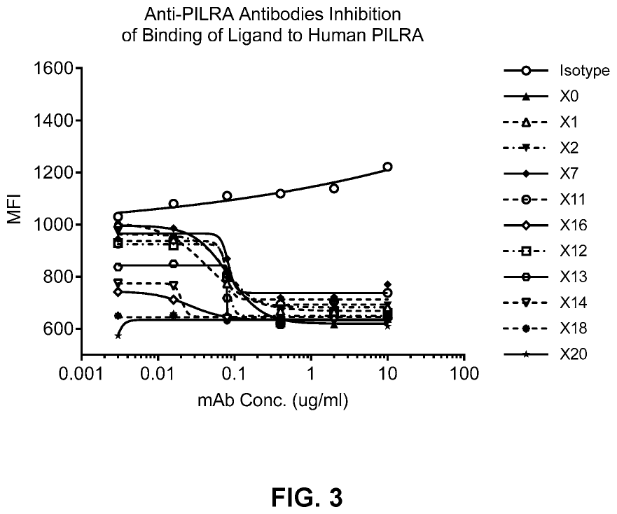

A dose response curve of antibodies X0 (PA-29), X1, X2, X7, X11, X12, X13, X14, X16, X18, and X20 was assessed for modulating the binding of Neural Proliferation Differentiation and Control Protein 1 (NPDC1) Fc to 293 cells ectopically expressing human PILRA (HEK293F human PILRA G78 overexpressing cells). Conditioned media samples containing antibodies were incubated with human PILRA overexpressing cells for 30 minutes at 4° C., and then 5 μg/mL NPDC1 Fc was added to this mixture. After 30 minutes of incubation at 4° C., the cells were washed and cell-bound NPDC1 Fc was detected with anti-human IgG Alexa Fluor 647. The cells were analyzed by flow cytometry on FACS Canto II with the percentage of NPDC1 Fc binding in the presence of conditioned media relative to NPDC1 Fc binding alone was calculated. FIG. 3 shows the results of the dose response curve with tested anti-PILRA antibodies. All tested antibodies decreased binding of NPDC1 to human PILRA.

### Example 8: Anti-PILRA Antibodies Binding Recombinant Cell Surface Human PILRA and PILRB, and Cynomolgus PILRA

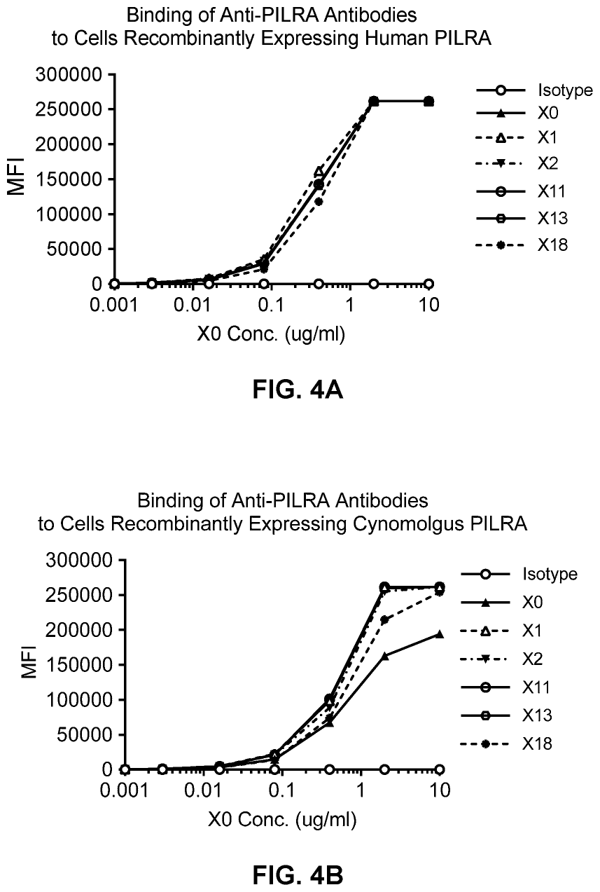

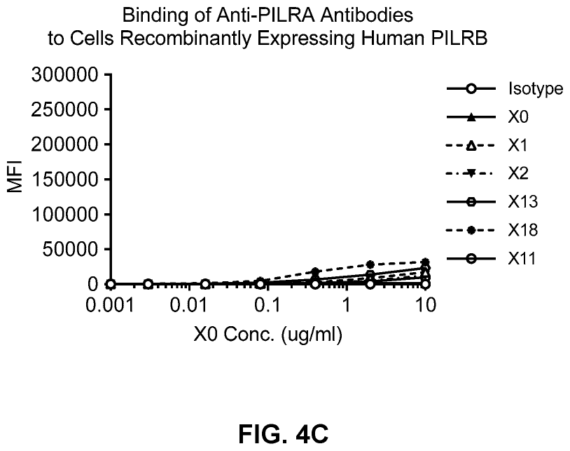

Conditioned media samples containing the tested anti-PILRA antibodies were assessed for binding to HEK293F cells overexpressing human PILRA (hA), human PILRB (hB), and cynomolgus PILRA (cA) by FACS. The conditioned media containing the tested antibodies were incubated with the cells for 30 min at 4° C., washed twice, and then detected with goat anti-human IgG secondary antibody (Alexa Fluor 647) at 1:1000 dilution. The cells were analyzed by flow cytometry on FACS Canto II, and for all cell lines, the reported MFI values were calculated for each conditioned media. The results are reported in Table 9 and FIGS. 4A-4C. These antibodies were evaluated for nonspecific binding using a baculovirus particle (BVP) assay (Jain T., Sun T., Durand S., Hall A., Houston N. R., Nett J. H., Sharkey B., Bobrowicz B., Caffry L, Yu Y., et al. Biophysical properties of the clinical-stage antibody landscape. Proc. Natl. Acad. Sci. USA. 2017; 114:944-949. Doi: 10.1073/pnas.1616408114.). The BVP scores are listed in Table 9, and the higher the score the more nonspecific binding that occurred.

TABLE 9

| | | | hA/hB | | | | | | | | | |
|---|---|---|---|---|---|---|---|---|---|---|---|---|
| | hA | hB | binding | cA | | Hu Mono | Binding | Binding | Binding | Binding | Blocking | MCP-1 |
| Tested | Kd | Kd | ratio on | Kd | BVP | DR EC50 | on G78 | on cA | on hu | on cyno | on G78 | release on |
| Antibodies | (nM) | (nM) | HEK293F | (nM) | score | (ug/ml) | OE | OE | mono | mono | OE | hu mono |
| X0 (PA-29) | 78.7 | weak | 108.3 | 192 | 2.95 | 0.7 | | | | | | |
| X1 | 0.4 | 126.0 | 56.7 | 5.3 | 3.15 | 0.02 | Similar | Binds | Binds | Binds | Similar | Lower |
| X2 | 0.8 | 216.0 | 71.1 | 8.9 | 3.65 | 0.1 | X0 | more | more | more | binding | concentration |
| X7 | 1.2 | weak | 63.0 | 2.0 | 16.65 | 0.9 | (PA-29) | strongly | strongly | strongly | affinity | of antibody |
| X11 | 1.6 | weak | 79.8 | 2.3 | 8.9 | 0.3 | | than X0 | than X0 | than X0 | as X0 | needed to |
| X12 | 0.1 | 36.7 | 14.4 | 0.3 | 2.8 | ~0.02 | | (PA-29) | (PA-29) | (PA-29) | (PA-29) | cause MCP-1 |
| X13 | 0.2 | 51.5 | 20.3 | 0.4 | 4.15 | ~0.0004 | | | | | | release than |
| X14 | 0.1 | 6.6 | 7.9 | 0.3 | 4.35 | ~0.02 | | | | | | X0 (PA-29) |
| X16 | N/A | N/A | 6.1 | N/A | 4 | 0.005 | | | | | | |
| X18 | 0.1 | 3.4 | 6.5 | 0.2 | 2.8 | ~0.02 | | | | | | |
| X20 | N/A | N/A | 6.7 | N/A | 6.5 | ~0.02 | | | | | | |

### Example 9: Downregulation of PILRA by anti-PILRA Antibodies

Anti-human PILRA antibodies were tested for the ability to downregulate cell surface PILRA on human monocytes (Donor 833). CD14+ monocytes were isolated from healthy human donors using a RosetteSep Human Monocyte Enrichment Cocktail kit (StemCell). Cells were treated with the tested antibodies for 24 hours at 37° C. PILRA surface expression was assessed by flow cytometry with an anti-PILRA polyclonal antibody (R&D).

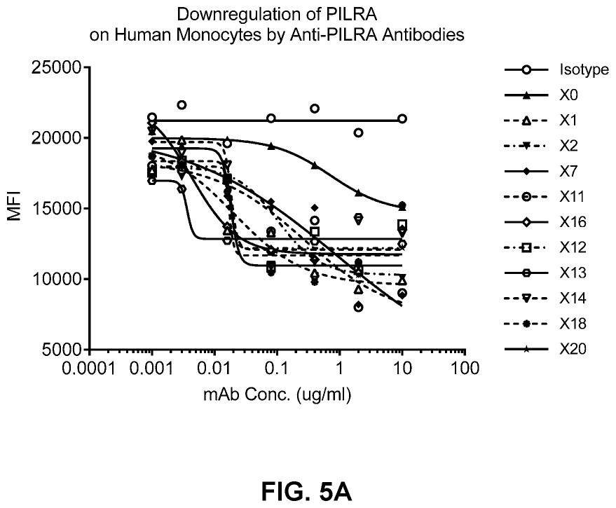

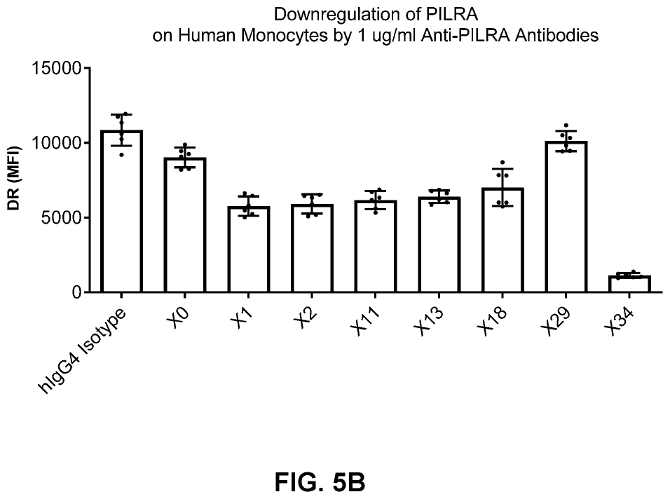

The results, shown in FIG. 5A, demonstrate that all 10 variants tested showed stronger downregulation of PILRA than the parental X0 (PA-29) antibody. A similar PILRA downregulation experiment was performed with antibodies XO (PA-29), X1, X2, X11, X13, X18, X29 (PA-43), and X34 (A6) (1 μg/mL) on human monocytes. FIG. 5B demonstrates that antibodies X1, X2, X11, X13, X18, and X34 (A6) all showed stronger downregulation of PILRA than the parental XO (PA-29) antibody.

### Example 10: Induction of MCP-1 by anti-PILRA Antibodies

Anti-human PILRA antibodies were tested for the ability to induce MCP-1 on human monocytes (Donor 833). CD14+ monocytes were isolated from healthy human donors using a RosetteSep Human Monocyte Enrichment Cocktail kit (StemCell). Cells were treated with the tested antibodies for 24 hours at 37° C. Supernatant was collected for MCP-1 detection by flow cytometry with LegendPlex human inflammatory 13-plex kit (Bioligand).

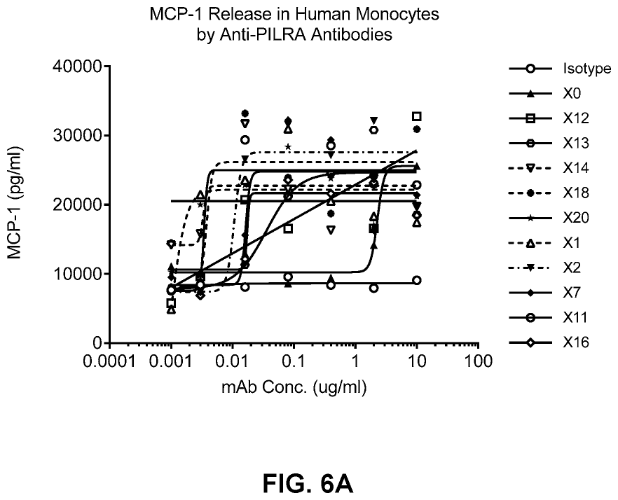

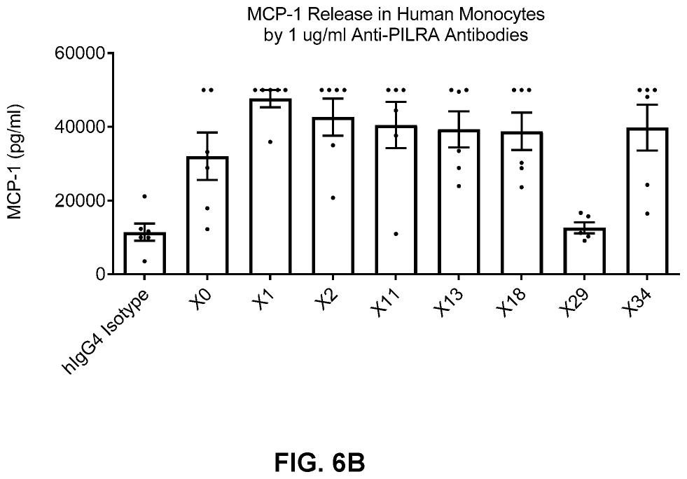

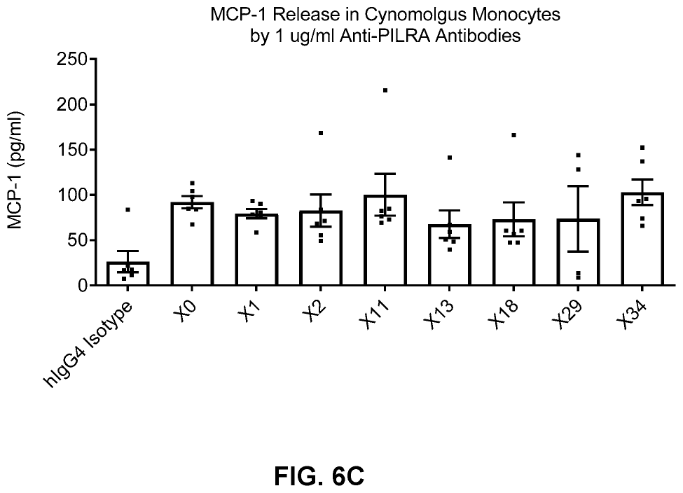

The results, shown in FIG. 6A, demonstrated that stronger MCP-1 inductions are observed in supernatant of monocytes treated with variants of X0 (PA-29), in dose response manner. A similar MCP-1 induction experiment was performed with antibodies X0 (PA-29), X1, X2, X11, X13, X18, X29 (PA-43), and X34 (A6) (1 μg/mL) on human and cynomolgus monocytes. FIG. 6B demonstrates that antibodies X1, X2, X11, X13, X18, and X34 (A6) all showed stronger MCP-1 induction on human monocytes than the parental X0 (PA-29) antibody. FIG. 6C demonstrates that the tested antibodies did not have reduced MCP-1 induction on cynomolgus monocytes compared to the parental XO (PA-29) antibody.

### Example 11: Activity in Mixed Lymphocyte Co-Culture Alone and in Combination with Anti-PD-L1

Anti-human PILRA antibodies were tested for the ability to activate CD3+T cells indirectly in allogeneic mixed lymphocyte co-culture (MLR) using human macrophages pairing with CD3+T cells from different donors.

To generate macrophages, CD14+ monocytes were isolated from healthy human donors using a RosetteSep Human Monocyte Enrichment Cocktail kit (StemCell) and differentiated at 37° C. and 5% CO2 for 7 days in RPMI/10% FBS containing 50 ng/ml M-CSF (R&D). CD3+T cells were isolated from healthy human donors using RosetteSep Human T-cell Enrichment Cocktail kit (StemCell).

Macrophages were treated with antibodies for 24 hours at 37° C. in X-VIVO media (Lonza). An anti-PD-L1 mIgG1 antibody (BM1-189-16) was included in the assay at a final concentration of 10 μg/mL. Carboxyfluorescein succinimidyl ester (CFSE)-labeled CD3+T cells were added to antibody treated macrophages at a 1:5 ratio of treated macrophages: CD3+T cells. The mixture was incubated at 37° C. and 5% CO₂ for 5 days. After 5 days of co-culture incubation, supernatant was collected for cytokine and chemokine detection using LegendPlex inflammatory 13-plex kits (Biolegend). T cells were collected to determine proliferation.

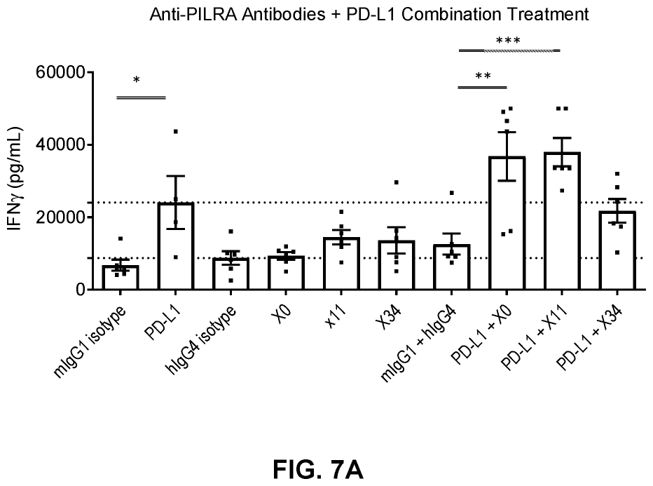

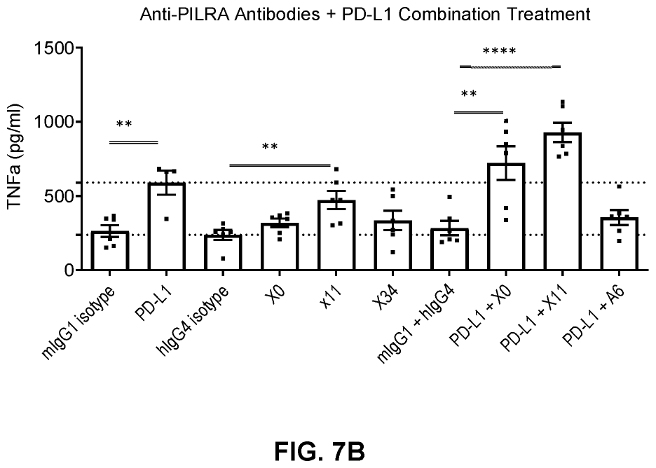

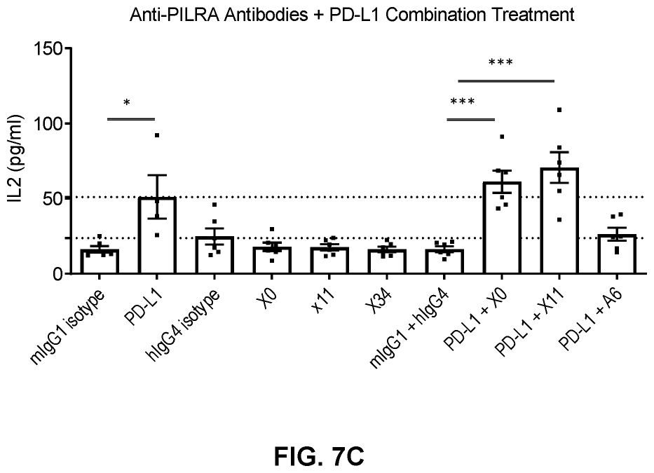

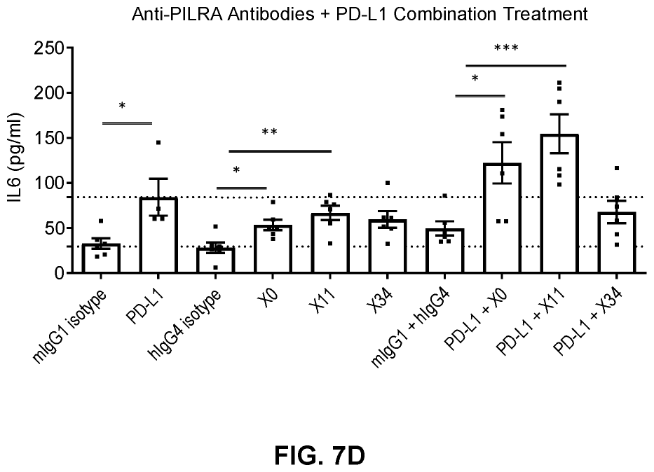

The results are shown in FIGS. 7A-7D. IFNg, TNFa, IL-2, and IL-6 inductions are observed on X0 (PA-29 parental)+anti-PD-L1 and X11+anti-PD-L1 treated macrophages/CD3+T co-culture compared to hIgG4 isotype treatment. FIGS. 7A-7D. In FIG. 7A, X0 (PA-29)+anti-PD-L1 and X11+anti-PD-L1 induced IFNg significantly more than X0 (PA-29) alone or X11 alone which shows that both X0 (PA-29) and X11 can activate T cells indirectly. In FIG. 7B, X0 (PA-29)+anti-PD-L1 and X11+anti-PD-L1 induced TNFalpha significantly more than X0 (PA-29) or X11 alone. Further, X11+anti-PD-L1 induced TNFalpha more significantly than X0 (PA-29)+anti-PD-L1 which shows that X11 is more potent than the X0 (PA-29) parental antibody. In FIG. 7C, X0 (PA-29)+anti-PD-L1 and X11+anti-PD-L1 induced IL2. In addition, X11+anti-PD-L1 induced IL2 significantly more than X0 (PA-29)+anti-PD-L1, which shows that X11 is more potent than the X0 (PA-29) parental antibody. No IL2 induction was observed with X0 (PA-29) or X11 alone. In FIG. 7D, X0 (PA-29)+anti-PD-L1 and X11+anti-PD-L1 induced IL6 significantly more than X0 (PA-29) or X11 alone. Further, X11+anti-PD-L1 induced IL6 more significantly than X0 (PA-29)+anti-PD-L1 which shows that X11 is more potent than the X0 (PA-29) parental antibody. In addition, X11 treatment alone induced IFNg, TNFa and IL-6 significantly more than treatment with X0 (PA-29) alone, which shows that X11 is more potent than the X0 (PA-29) parental antibody. See FIGS. 7A, 7B, and 7D. In addition, IFNg, TNFa and IL-6 inductions are observed on X11 single treatment, which shows that X11 is more potent than the X0 (PA-29) parental antibody. See FIGS. 7A, 7B, and 7D.

The invention is not to be limited in scope by the specific aspects described herein. Indeed, various modifications of the invention in addition to those described will become apparent to those skilled in the art from the foregoing description and accompanying figures. Such modifications are intended to fall within the scope of the appended claims.

All references (e.g., publications or patents or patent applications) cited herein are incorporated herein by reference in their entirety and for all purposes to the same extent as if each individual reference (e.g., publication or patent or patent application) was specifically and individually indicated to be incorporated by reference in its entirety for all purposes.

Other embodiments are within the following claims.

What is claimed:

1. An isolated antibody or antigen-binding fragment thereof that specifically binds to human PILRA and comprises the heavy chain variable region (VH) complementarity determining region (CDR) 1, VH CDR2, VH CDR3 and light chain variable region (VL) CDR1, CDR2, and CDR3 sequences of:
(a) SEQ ID NOs: 4-6, 32, 14, and 26, respectively;
(b) SEQ ID NOs: 10, 5, 11, and 13-15, respectively;
(c) SEQ ID NOs: 10, 5, 12, and 13-15, respectively;
(d) SEQ ID NOs: 4-6, 13, 14, and 25, respectively;
(e) SEQ ID NOs: 4-6, 19, 14, and 25, respectively;
(f) SEQ ID NOs: 4-6, 20, 14, and 25, respectively;
(g) SEQ ID NOs: 4-6, 21, 14, and 25, respectively;
(h) SEQ ID NOs: 4-6, 22, 14, and 25, respectively;
(i) SEQ ID NOs: 4-6, 27, 14, and 25, respectively;
(j) SEQ ID NOs: 4-6, 20, 14, and 26, respectively;
(k) SEQ ID NOs: 4-6, 22, 14, and 26, respectively;
(l) SEQ ID NOs: 10, 5, 11, 13, 14, and 25, respectively;
(m) SEQ ID NOs: 10, 5, 12, 13, 14, and 25, respectively;
(n) SEQ ID NOs: 10, 5, 11, 19, 14, and 25, respectively;
(o) SEQ ID NOs: 10, 5, 11, 20, 14, and 25, respectively;
(p) SEQ ID NOs: 10, 5, 11, 21, 14, and 25, respectively;
(q) SEQ ID NOs: 10, 5, 11, 22, 14, and 25, respectively;
(r) SEQ ID NOs: 10, 5, 11, 33, 14, and 25, respectively;
(s) SEQ ID NOs: 10, 5, 12, 22, 14, and 26, respectively;
(t) SEQ ID NOs: 10, 5, 12, 32, 14, and 26, respectively;
(u) SEQ ID NOs: 10, 5, 6, 13, 14, and 25, respectively;
(v) SEQ ID NOs: 10, 5, 6, 13, 14, and 26, respectively;
(w) SEQ ID NOs: 10, 5, 6, 19, 14, and 15, respectively;
(x) SEQ ID NOs: 10, 5, 6, 21, 14, and 15, respectively;
(y) SEQ ID NOs: 4, 5, 12, 13, 14, and 25, respectively;
(z) SEQ ID NOs: 4, 5, 12, 13, 14, and 26, respectively;
(aa) SEQ ID NOs: 4, 5, 12, 19, 14, and 15, respectively;
(bb) SEQ ID NOs: 4, 5, 12, 21, 14, and 15, respectively;
(cc) SEQ ID NOs: 4-6, 21, 14, and 141, respectively;
(dd) SEQ ID NOs: 4-6, 140, 14, and 141, respectively;
(ee) SEQ ID NOs: 10, 5, 12, 20, 14, and 141, respectively; or
(ff) SEQ ID NOs: 110, 5, 12, 140, 14, and 141, respectively.

2. The antibody or antigen-binding fragment of claim 1, wherein the antibody or antigen-binding comprises a heavy chain variable region and/or a light chain variable region comprising the amino acid sequences of:
(a) SEQ ID NOs: 28 and 71, respectively;
(b) SEQ ID NOs: 30 and 60, respectively;
(c) SEQ ID NOs: 31 and 60, respectively;
(d) SEQ ID NOs: 28 and 63, respectively;
(e) SEQ ID NOs: 28 and 64, respectively;
(f) SEQ ID NOs: 28 and 65, respectively;
(g) SEQ ID NOs: 28 and 66, respectively;
(h) SEQ ID NOs: 28 and 67, respectively;
(i) SEQ ID NOs: 28 and 68, respectively;
(j) SEQ ID NOs: 28 and 69, respectively;
(k) SEQ ID NOs: 28 and 70, respectively;
(l) SEQ ID NOs: 30 and 63, respectively;
(m) SEQ ID NOs: 31 and 63, respectively;
(n) SEQ ID NOs: 30 and 64, respectively;
(o) SEQ ID NOs: 30 and 65, respectively;
(p) SEQ ID NOs: 30 and 66, respectively;
(q) SEQ ID NOs: 30 and 67, respectively;
(r) SEQ ID NOs: 30 and 78, respectively;
(s) SEQ ID NOs: 31 and 70, respectively;
(t) SEQ ID NOs: 31 and 71, respectively;
(u) SEQ ID NOs: 50 and 63, respectively;
(v) SEQ ID NOs: 50 and 82, respectively;
(w) SEQ ID NOs: 50 and 83, respectively;
(x) SEQ ID NOs: 50 and 84, respectively;
(y) SEQ ID NOs: 54 and 63, respectively;
(z) SEQ ID NOs: 54 and 82, respectively;
(aa) SEQ ID NOs: 54 and 83, respectively;
(bb) SEQ ID NOs: 54 and 84, respectively
(cc) SEQ ID NOs: 28 and 142, respectively;
(dd) SEQ ID NOs: 28 and 143, respectively;
(ee) SEQ ID NOs: 31 and 69, respectively; or
(ff) SEQ ID NOs: 31 and 143, respectively.

3. The antibody or antigen-binding fragment thereof of claim 1 or 2, wherein the antibody or antigen-binding fragment binds to human PILRA with a binding affinity between about 0.2 nM to 12 nM.

4. The antibody or antigen-binding fragment thereof of any one of claims 1-3, wherein the antibody or antigen-binding fragment blocks binding of human PILRA to one or more of its ligands.

5. The antibody or antigen-binding fragment thereof of claim 4, wherein the ligand is NPDC1.

6. The antibody or antigen-binding fragment thereof of any one of claims 1-5, wherein the antibody or antigen-binding fragment binds to cynomolgus PILRA.

7. The antibody or antigen-binding fragment thereof of claim 6, wherein the antibody or antigen-binding fragment binds to cynomolgus PILRA with a binding affinity between about 0.1 nM to 2 nM.

8. The antibody or antigen-binding fragment thereof of any one of claims 1-7, wherein the antibody or antigen-binding fragment binds to human PILRB with a binding affinity between about 2 nM to 200 nM.

9. The antibody or antigen-binding fragment thereof of any one of claims 1-8, wherein the antibody or antigen-binding fragment binds to cynomolgus PILRB with a binding affinity between about 0.1 nM to 2 nM.

10. The antibody or antigen-binding fragment thereof of any one of claims 1-9, wherein the antibody or antigen-binding fragment blocks binding of soluble human PILRA binding to T cells.

11. The antibody or antigen-binding fragment thereof of any one of claims 1-10, wherein the antibody or antigen-binding fragment thereof downregulates cell surface human PILRA.

12. The antibody or antigen-binding fragment there of any one of claims 1-11, wherein the antibody or antigen-binding fragment thereof downregulates cell surface human PILRA by about 20% to 35% more than antibody X0.

13. The antibody or antigen-binding fragment there of any one of claims 1-11, wherein the antibody or antigen-binding fragment thereof downregulates cell surface human PILRA by at least 20% more than antibody X0.

14. The antibody or antigen-binding fragment there of any one of claims 1-12, wherein the antibody or antigen-binding fragment thereof downregulates cell surface human PILRA by at least 25% more than antibody X0.

15. The antibody or antigen-binding fragment there of any one of claims 1-12, wherein the antibody or antigen-binding fragment thereof downregulates cell surface human PILRA by at least 30% more than antibody X0.

16. The antibody or antigen-binding fragment there of any one of claims 1-11, wherein the antibody or antigen-binding fragment thereof downregulates cell surface human PILRA by at least 35% more than antibody X0.

17. The antibody or antigen-binding fragment thereof of any one of claims 1-16, wherein the antibody or antigen-binding fragment agonizes human PILRB.

18. The antibody or antigen-binding fragment thereof of any one of claims 1-17, wherein the antibody or antigen-binding fragment binds to the G78 variant of human PILRA, optionally wherein the antibody or antigen-binding fragment binds to the G78 variant of human PILRA with an EC50 of no more than 4 μg/ml, no more than 3.5 μg/ml, no more than 3 μg/ml, no more than 2.5 μg/ml, no more than 2 μg/ml, no more than 1.5 μg/ml, or no more than 1 μg/ml.

19. The antibody or antigen-binding fragment thereof of any one of claims 1-18, wherein the antibody or antigen-binding fragment binds to cynomolgus PILRA with an EC50 of no more than 20 μg/ml, no more than 15 μg/ml, no more than 10 μg/ml, no more than 5 μg/ml, no more than 3 μg/ml, or no more than 1 g/ml.

20. The antibody or antigen-binding fragment thereof of any one of claims 1-19, wherein the antibody or antigen-binding fragment decreases binding of human PILRA to one or more of its ligands by at least 90%.

21. The antibody or antigen-binding fragment thereof of any one of claims 1-20, wherein the antibody or antigen-binding fragment thereof is capable of producing a dose-dependent induction of MCP-1 in monocytes.

22. The antibody or antigen-binding fragment thereof of any one of claims 1-21, wherein the antibody or antigen-binding fragment thereof is capable of inducing release of at least 38,000 μg/mL MCP-1, wherein the antibody or antigen-binding fragment is present at a concentration of 1 μg/mL.

23. The antibody or antigen-binding fragment thereof of any one of claims 1-22, wherein the antibody or antigen-binding fragment thereof is capable of inducing more MCP-1 in monocytes than antibody X0.

24. The antibody or antigen-binding fragment thereof of any one of claims 1-22, wherein the antibody or antigen-binding fragment thereof is capable of inducing 10% to 50% more MCP-1 in monocytes than antibody X0.

25. The antibody or antigen-binding fragment thereof of any one of claims 1-22, wherein the antibody or antigen-binding fragment thereof is capable of inducing at least 10% more MCP-1 in monocytes than antibody X0.

26. The antibody or antigen-binding fragment thereof of any one of claims 1-24, wherein the antibody or antigen-binding fragment thereof is capable of inducing at least 20% more MCP-1 in monocytes than antibody X0.

27. The antibody or antigen-binding fragment thereof of any one of claims 1-24, wherein the antibody or antigen-binding fragment thereof is capable of inducing at least 30% more MCP-1 in monocytes than antibody X0.

28. The antibody or antigen-binding fragment thereof of any one of claims 1-24, wherein the antibody or antigen-binding fragment thereof is capable of inducing at least 40% more MCP-1 in monocytes than antibody X0.

29. The antibody or antigen-binding fragment thereof of any one of claims 1-24, wherein the antibody or antigen-binding fragment thereof is capable of inducing at least 50% more MCP-1 in monocytes than antibody X0.

30. The antibody or antigen-binding fragment thereof of any one of claims 1-29, wherein the antibody or antigen-binding fragment thereof is capable of producing a dose-dependent induction of IL-8 in monocytes.

31. The antibody or antigen-binding fragment thereof of any one of claims 1-30, wherein the antibody or antigen-binding fragment thereof is capable of inducing IFNg in macrophages.

32. The antibody or antigen-binding fragment thereof of any one of claims 1-31, wherein the antibody or antigen-binding fragment thereof is capable of inducing TNFa in macrophages.

33. The antibody or antigen-binding fragment thereof of any one of claims 1-32, wherein the antibody or antigen-binding fragment thereof is capable of inducing IL-6 in macrophages.

34. The antibody or antigen-binding fragment thereof of any one of claims 1-33, wherein the antibody or antigen-binding fragment thereof in combination with an antagonist of PD-L1 is capable of inducing IFNg in macrophages.

35. The antibody or antigen-binding fragment thereof of any one of claims 1-34, wherein the antibody or antigen-binding fragment thereof in combination with an antagonist of PD-L1 is are capable of inducing TNFa in macrophages.

36. The antibody or antigen-binding fragment thereof of any one of claims 1-35, wherein the antibody or antigen-binding fragment thereof in combination with an antagonist of PD-L1 agent is capable of inducing IL-2 in macrophages.

37. The antibody or antigen-binding fragment thereof of any one of claims 1-36, wherein the antibody or antigen-binding fragment thereof in combination with an antagonist of PD-L1 is capable of inducing IL-6 in macrophages.

38. The antibody or antigen-binding fragment thereof of any one of claims 34-37, wherein the antagonist of PD-L1 is an anti-PD-L1 antibody or antigen-binding fragment thereof, optionally wherein the anti-PD-L1 antibody or antigen-binding fragment thereof is selected from the group consisting of atezolizumab, durvalumab (MEDI4736), BMS-936559, MSB0010718C and rHigM12B7

39. The antibody or antigen-binding fragment thereof of any one of claims 1-38, wherein the antibody or antigen-binding fragment comprises a heavy chain constant region and a light chain constant region.

40. The antibody or antigen-binding fragment thereof of claim 39, wherein the heavy chain constant region is an isotype selected from the group consisting of human IgG1, IgG2, IgG3, and IgG4 isotypes.

41. The antibody or antigen-binding fragment thereof of any one of claims 1-40, which comprises an Fc domain that is engineered to reduce effector function.

42. The antibody or antigen-binding fragment thereof of any one of claims 1-41, wherein the antibody or antigen-binding fragment comprises a heavy chain constant region and a light chain constant region, wherein the heavy chain constant region is a human IgG₁ heavy chain constant region, and wherein the light chain constant region is a human IgG_{κ} light chain constant region.

43. The antibody or antigen-binding fragment thereof of any one of claims 1-42, wherein the antibody or antigen-binding fragment is a monoclonal antibody.

44. The antibody or antigen-binding fragment thereof of any one of claims 1-43, wherein the antibody or antigen-binding fragment thereof is a murine, chimeric, humanized, or human antibody or antigen-binding fragment thereof.

45. The antibody or antigen binding fragment thereof of any one of claims 1-44, which is a full length antibody.

46. The antibody or antigen binding fragment thereof of any one of claims 1-44, which is an antigen binding fragment.

47. The antigen binding fragment of claim 46, wherein the antigen binding fragment is a Fab, Fab′, F(ab′)₂, single chain Fv (scFv), disulfide linked Fv, V-NAR domain, IgNar, intrabody, IgGΔCH2, minibody, F(ab′)₃, tetrabody, triabody, diabody, single-domain antibody, DVD-Ig, Fcab, mAb², (scFv)₂, or scFv-Fc.

48. The antibody or antigen-binding fragment thereof of any one of claims 1-47, further comprising a detectable label.

49. An antibody or antigen-binding fragment thereof that binds to the same epitope as the antibody or antigen-binding fragment thereof of any one of claims 1-47.

50. An antibody or antigen-binding fragment thereof that competitively inhibits binding to human PILRA of the antibody or antigen-binding fragment thereof of any one of claims 1-47.

51. An isolated polynucleotide comprising a nucleic acid molecule encoding the heavy chain variable region or heavy chain of the antibody or antigen-binding fragment thereof of any one of claims 1-50.

52. The isolated polynucleotide of claim 51, wherein the nucleic acid molecule encodes the VH of any one of SEQ ID NOs: 28, 30, 31, 50, or 54.

53. An isolated polynucleotide comprising a nucleic acid molecule encoding the light chain variable region or light chain of the antibody or antigen-binding fragment thereof of any one of claims 1-50.

54. The isolated polynucleotide of claim 53, wherein the nucleic acid molecule encodes the VL of any one of SEQ ID NOs: 60, 63-71, 78, 82-84, 142, or 143.

55. An isolated polynucleotide comprising a nucleic acid molecule encoding the heavy chain variable region or heavy chain of the antibody or antigen-binding fragment thereof of any one of claims 1-50 and the light chain variable region or light chain of the antibody or antigen-binding fragment thereof of any one of claims 1-50.

56. An isolated vector comprising the polynucleotide of any one of claims 51-55.

57. A host cell comprising (a) the polynucleotide of any one of claims 51-55 (b) the vector of claim 56, or (c) a first vector comprising the polynucleotide of claim 51 or 52 and a second vector comprising the polynucleotide of claim 53 or 54.

58. The host cell of claim 57, which is selected from the group consisting of E. coli, Pseudomonas, Bacillus, Streptomyces, yeast, CHO, YB/20, NSO, PER-C6, HEK-293T, NIH-3T3, HeLa, BHK, Hep G2, SP2/0, R1.1, B-W, L-M, COS 1, COS 7, BSC1, BSC40, BMT10 cell, plant cell, insect cell, and human cell in tissue culture, optionally wherein the host cell is a HEK-293 cell.

59. A method of producing an antibody or antigen-binding fragment thereof that binds to human PILRA comprising culturing the host cell of claim 57 or 58 so that the nucleic acid molecule is expressed and the antibody or antigen-binding fragment thereof is produced, optionally wherein the method further comprises isolating the antibody or antigen-binding fragment thereof from the culture.

60. An isolated antibody or antigen-binding fragment thereof that specifically binds to human PILRA and is encoded by the polynucleotide of any one of claims 51-55 or produced by the method of claim 59.

61. A pharmaceutical composition comprising the antibody or antigen-binding fragment thereof of any one of claims 1-50 and 60 and a pharmaceutically acceptable excipient.

62. A method for downregulating cell surface PILRA comprising contacting a cell expressing PILRA on its surface with the antibody or antigen-binding fragment thereof of any one of claims 1-50 and 60 or the pharmaceutical composition of claim 61.

63. A method for inhibiting binding of PILRA to a PILRA ligand comprising contacting PILRA with the antibody or antigen-binding fragment thereof of any one of claims 1-50 and 60 or the pharmaceutical composition of claim 61 in the presence of the PILRA ligand, optionally wherein the PILRA and/or the PILRA ligand is expressed on a cell.

64. The method of claim 63, wherein the PILRA ligand is NPDC1.

65. The method of claim 63 or 64, wherein the PILRA ligand is expressed on a T cell.

66. A method for increasing myeloid cell activation comprising contacting the myeloid cell with the antibody or antigen-binding fragment thereof of any one of claims 1-50 and 60 or the pharmaceutical composition of claim 61.

67. The method of claim 66, wherein the myeloid cell activation is Fc receptor-mediated.

68. A method for promoting myeloid cell differentiation comprising contacting the myeloid cell with the antibody or antigen-binding fragment thereof of any one of claims 1-50 and 60 or the pharmaceutical composition of claim 61.

69. A method for increasing myeloid cell production of MIP1b comprising contacting the myeloid cell with the antibody or antigen-binding fragment thereof of any one of claims 1-50 and 60 or the pharmaceutical composition of claim 61.

70. The method of any one of claims 62-69, wherein the contacting is in vitro.

71. The method of any one of claims 62-69, wherein the contacting is in a subject.

72. A method of treating cancer in a patient, the method comprising administering to the patient a therapeutically effective amount of the antibody or antigen binding fragment thereof of any one of claims 1-50 and 60 or the pharmaceutical composition of claim 61.

73. The method of claim 72, wherein the cancer is a solid tumor in which myeloid cells have infiltrated the tumor microenvironment.

74. The method of claim 72 or 73, wherein the cancer is selected from the group consisting of: glioblastoma, head and neck cancer, kidney cancer (optionally wherein the kidney cancer is kidney clear cell cancer), pancreatic cancer, breast cancer, ovarian cancer, sarcoma, colorectal cancer, lung cancer, melanoma, bladder cancer, liver cancer, and uterine cancer.

75. The method of any one of claims 72-74, further comprising administering an antagonist of an inhibitory immune checkpoint molecule, optionally wherein the immune checkpoint molecule is PD-1 or PD-L1.

76. The method of claim 75, wherein the antagonist is an antagonist of PD-1, which is an anti-PD-1 antibody or antigen-binding fragment thereof, optionally wherein the anti-PD-1 antibody or antigen-binding fragment thereof is selected from the group consisting of nivolumab, pembrolizumab, MEDI-0680 (AMP-514), camrelizumab (SHR-1210), tislelizumab (BGB-A317), and spartalizumab (NPVPDR001, NVS240118, PDR001).

77. The method of claim 75, wherein the antagonist is an antagonist of PD-L1, which is an anti-PD-L1 antibody or antigen-binding fragment thereof, optionally wherein the anti-PD-L1 antibody or antigen-binding fragment thereof is selected from the group consisting of atezolizumab, durvalumab (MEDI4736), BMS-936559, MSB0010718C and rHigM12B7.

78. The method of any one of claims 75-77, wherein the antibody or antigen-binding fragment thereof that specifically binds to human PILRA and the antagonist of the inhibitory immune checkpoint molecule are administered simultaneously.

79. The method of any one of claims 75-77, wherein the antibody or antigen-binding fragment thereof that specifically binds to human PILRA and the antagonist of the inhibitory immune checkpoint molecule are administered sequentially.

80. A method of treating a disease or condition in which myeloid cells are dysfunctional or deficient in a patient, the method comprising administering to the patient a therapeutically effective amount of the antibody or antigen binding fragment thereof of any one of claims 1-50 and 60 or the pharmaceutical composition of claim 61, optionally further administering an antagonist of PD-L1.

81. A method of treating a neurodegenerative disease in a patient, the method comprising administering to the patient a therapeutically effective amount of the antibody or antigen binding fragment thereof of any one of claims 1-50 and 60 or the pharmaceutical composition of claim 61, optionally further comprising administering an antagonist of PD-L1.

82. The method of claim 81, wherein the neurodegenerative disease is selected from the group consisting of: Alzheimer's disease, dementia, frontotemporal dementia (FTD), vascular dementia, mild cognitive impairment, Parkinson's disease, amyotrophic lateral sclerosis (ALS), Huntington's disease, Tauopathy disease, multiple sclerosis (MS), immune-mediated neuropathies (such as neuropathic pain), Nasu-Hakola disease, pediatric-onset leukoencephalopathy, adult-onset leukoencephalopathy with axonal spheroids and pigmented glia (ALSP), and limbic-predominant age-related TDP43 encephalopathy (LATE).

83. The method of claim 81, wherein the neurodegenerative disease is Alzheimer's disease, optionally wherein the patient carries the G78 variant of PILRA.

84. The method of claim 83, wherein the method increases MCP-1, IL-8, IFNg, TNFa, IL-6, and/or MIP1b in the patient.

85. A method of increasing MCP-1 in a monocyte comprising contacting the monocyte with the antibody or antigen-binding fragment thereof of any one of claims 1-50 and 60 or the pharmaceutical composition of claim 61, optionally further comprising contacting an antagonist of PD-L1 with the monocyte.

86. The method of claim 85, wherein the monocyte is in vitro.

87. The method of claim 85, wherein the monocyte is in a subject.

88. The method of claim 87, wherein the subject is a human subject.

89. The method of any one of claims 85-88, wherein the contacting comprises administering the antibody, antigen-binding fragment thereof, or pharmaceutical composition to a subject.

90. A method of increasing IL-8 in a monocyte comprising contacting the monocyte with the antibody or antigen-binding fragment thereof of any one of claims 1-50 and 60 or the pharmaceutical composition of claim 61, optionally further contacting an antagonist of PD-L1 with the monocyte.

91. The method of claim 90, wherein the monocyte is in vitro.

92. The method of claim 90, wherein the monocyte is in a subject.

93. The method of claim 92, wherein the subject is a human subject.

94. The method of any one of claims 90-93, wherein the contacting comprises administering the antibody, antigen-binding fragment thereof, or pharmaceutical composition to a subject.

95. A method of increasing IFNg in a macrophage comprising contacting the macrophage with the antibody or antigen-binding fragment thereof of any one of claims 1-50 and 60 or the pharmaceutical composition of claim 61, optionally further contacting an antagonist of PD-L1 with the macrophage.

96. The method of claim 95, wherein the macrophage is in vitro.

97. The method of claim 95, wherein the macrophage is in a subject.

98. The method of claim 96, wherein the subject is a human subject.

99. The method of any one of claims 95-98, wherein the contacting comprises administering the antibody, antigen-binding fragment thereof, or pharmaceutical composition to a subject.

100. A method of increasing TNFa in a macrophage comprising contacting the macrophage with the antibody or antigen-binding fragment thereof of any one of claims 1-50 and 60 or the pharmaceutical composition of claim 61, optionally further contacting an antagonist of PD-L1 with the macrophage.

101. The method of claim 100, wherein the macrophage is in vitro.

102. The method of claim 100, wherein the macrophage is in a subject.

103. The method of claim 102, wherein the subject is a human subject.

104. The method of any one of claims 100-103, wherein the contacting comprises administering the antibody, antigen-binding fragment thereof, or pharmaceutical composition to a subject.

105. A method of increasing IL-2 in a macrophage comprising contacting the macrophage with the antibody or antigen-binding fragment thereof of any one of claims 1-50 and 60 or the pharmaceutical composition of claim 61, optionally further contacting an antagonist of PD-L1 with the macrophage.

106. The method of claim 105, wherein the macrophage is in vitro.

107. The method of claim 105, wherein the macrophage is in a subject.

108. The method of claim 107, wherein the subject is a human subject.

109. The method of any one of claims 105-108, wherein the contacting comprises administering the antibody, antigen-binding fragment thereof, or pharmaceutical composition to a subject.

110. A method of increasing IL-6 in a macrophage comprising contacting the macrophage with the antibody or antigen-binding fragment thereof of any one of claims 1-50 and 60 or the pharmaceutical composition of claim 61, optionally further contacting an antagonist of PD-L1 with the macrophage.

111. The method of claim 110, wherein the macrophage is in vitro.

112. The method of claim 110, wherein the macrophage is in a subject.

113. The method of claim 112, wherein the subject is a human subject.

114. The method of any one of claims 110-113, wherein the contacting comprises administering the antibody, antigen-binding fragment thereof, or pharmaceutical composition to a subject.

115. The method of any one of claims 85-114, wherein the antagonist of PD-L1 is an anti-PD-L1 antibody or antigen-binding fragment thereof, optionally wherein the anti-PD-L1 antibody or antigen-binding fragment thereof is selected from the group consisting of atezolizumab, durvalumab (MEDI4736), BMS-936559, MSB0010718C and rHigM12B7.

116. A method of activating the innate immune system in a patient, the method comprising administering to the patient an effective amount of the antibody or antigen binding fragment thereof of any one of claims 1-50 and 60 or the pharmaceutical composition of claim 61.

117. A method for detecting PILRA in a sample comprising contacting the sample with the antibody or antigen-binding fragment thereof of any one of claims 1-50 and 60 or the pharmaceutical composition of claim 61.

118. The method of claim 117, wherein the sample is obtained from a human subject, optionally wherein the sample is a cancer sample.

119. The antibody or antigen-binding fragment thereof of any one of claims 1-50 and 60 or the pharmaceutical composition of claim 61 for use in the method of any one of claims 62-116.

120. The antibody or antigen-binding fragment thereof of any one of claims 1-50 and 60 or the pharmaceutical composition of claim 61 for the manufacture of a medicament for use in the method of any one of claims 62-85, 87-90, 92-95, 97-100, 102-105, 107-110, and 112-116.

121. Use of the antibody or or antigen-binding fragment thereof of any one of claims 1-50 and 60 or the pharmaceutical composition of claim 61 in the method of any one of claims 62-116.
